SEQUENCE LISTING (170 sequences)

SEQ ID NO: 1 - protein, 26 aa, Artificial Sequence
  VARIANT 22: Xaa = Thr or Gln or Ser
  VARIANT 2,3,4,5,6,7,8,11,14,16,17,20,24,25: Xaa = any amino acid
  VARIANT 10,19: Xaa = Lys or Arg
  VARIANT 12,13: Xaa = Tyr or Phe
  VARIANT 15: Xaa = Asn or Asp
  VARIANT 18, 21: Xaa = Thr or Ser
  Consensus Sequence of WW domains
WXXXXXXXGXXXXXXXXXXXXXWXXP

SEQ ID NO: 2 - protein, 5 aa, Artificial Sequence
  Homologous motif in WBP-1 and WBP-2
PPPPY

SEQ ID NO: 3 - protein, 4 aa, Artificial Sequence
  Conserved motif of PDZ domains
GLGF

SEQ ID NO: 4 - protein, 4 aa, Artificial Sequence
  C-terminal consensus motif of some of the ion channels and surface receptors
  VARIANT 1, 3: Xaa = Any Amino Acid
  VARIANT 2: Xaa = Ser or Thr
  VARIANT 1, 2, 3: Xaa = Any Amino Acid
  VARIANT 1, 2, 3: Xaa = Any Amino Acid
XXXV

SEQ ID NO: 5 - protein, 4 aa, Artificial Sequence
  subunit of inward K channel protein
ESAI

SEQ ID NO: 6 - protein, 4 aa, Unknow
  Binding site of peptide ligand
QTSV

SEQ ID NO: 7 - protein, 4 aa, Unknow
  Binding site of peptide ligand
ESIV

SEQ ID NO: 8 - protein, 5 aa, Unknow
  C-teminal end of Fas receptor
IQSLV

SEQ ID NO: 9 - protein, 85 aa, Homo Sapiens
REPRRIVIHRGSTGLGFNIVGGEDGEGIFISFILAGGPANLSGELRKGDQILSVNGVDLRNASHEQAAIALKNAGQTVTIIAQYK

SEQ ID NO: 10 - protein, 87 aa, Homo Sapiens
GELHMIELEKGHSGLGLSLAGNKDRSRMSVFIVGIDPNGAAGKDGRLQIADELLEINGQILYGRSHQNASSIIKCAPSKVKIIFIRN

SEQ ID NO: 11 - protein, 85 aa, Homo Sapiens
KNVQHLELPKDQGGLGIAISEEDTLSGVIIKSLTEHGVAATDGRLKVGDQILAVDDEIVVGYPIEKFISLLKTAKMTVKLTIHAE

SEQ ID NO: 12 - protein, 86 aa, Homo Sapiens
GKFIHTKLRKSSRGFGFTVVGGDEPDEFLQIKSLVLDGPAALDGKMETGDVIVSVNDTCVLGHTHAQVVKIFQSIPIGASVGPELC

SEQ ID NO: 13 - protein, 82 aa, Homo Sapiens
PELITVHIVKGPMGFGFTIADSPGGGGQRVKQIVDSPRCRGLKEGDLIVEVNKKNVQALTHNQVVDMLVECPKGSEVTLLVQ

SEQ ID NO: 14 - protein, 86 aa, Homo Sapiens
YQEQDIFLWRKETGFGFRILGGNEPGEPIYIGHIVPLGAADTDGRLRSGDELICVDGTPVIGKSHQLVVQLMQQAAKQGHVNLTVR

SEQ ID NO: 15 - protein, 99 aa, Homo Sapiens
VQPYDVEIRRGENEGFGFVIVSSVSRPEAGTTFGNACVAMPHKIGRIIEGSPADRCGKLKVGDRILAVNGCSITNKSHSDIVNLIKEAGNTVTLRIISW

SEQ ID NO: 16 - protein, 87 aa, Homo Sapiens
GQEMIIEISKGRSGLGLSIVGGKDTPLNAIVIHEVYEEGAAARDGRLWAGDQILEVNGVDLRNSSHEEAITALRQTPQKVRLVVYRD

SEQ ID NO: 17 - protein, 85 aa, Homo Sapiens
EIFPVDLQKKAGRGLGLSIVGKRNGSGVFISDIVKGGAADLDGRLIQGDQILSVNGEDMRNASQETVATILKCAQGLVQLEIGRL

SEQ ID NO: 18 - protein, 86 aa, Homo Sapiens
GTFLSTTLKKSNMGFGFTIIGGDEPDEFLQVKSVIPDGPAAQDGKMETGDVIVYINEVCVLGHTHADVVKLFQSVPIGQSVNLVLC

SEQ ID NO: 19 - protein, 80 aa, Homo Sapiens
AELMTLTIVKGAQGFGFTIADSPTGQRVKQILDIQGCPGLCEGDLIVEINQQNVQNLSHTEVVDILKDCPIGSETSLIIH

SEQ ID NO: 20 - protein, 86 aa, Homo Sapiens
YKELDVHLRRMESGFGFRILGGDEPGQPILIGAVIAMGSADRDGRLHPGDELVYVDGIPVAGKTHRYVIDLMHHAARNGQVNLTVR

SEQ ID NO: 21 - protein, 92 aa, Homo Sapiens
QTSDVVIHRKENEGFGFVIISSLNRPESGSTITVPHKIGRIIDGSPADRCAKKVGDRILAVNGQSIINMPHADIVKLIKDAGLSVTLRIIPQ

SEQ ID NO: 22 - protein, 86 aa, Homo Sapiens
GVLVRASLKKSTMGFGFTIIGGDRPDEFLQVKNVLKDGPAAQDGKIAPGDVIVDINGNCVLGHTHADVVQMFQLVPVNQYVNLTLC

SEQ ID NO: 23 - protein, 80 aa, Homo Sapiens
PELVTIPLIKGPKGFGFAIADSPTGQKVKMILDSQWCQGLQKGDIIKEIYHQNVQNLTHLQVVQVLKQFPVGADVPLLIL

SEQ ID NO: 24 - protein, 91 aa, Homo Sapiens
EEELTLTILRQTGGLGISIAGGKGSTPYKGDDEGIFISRVSEEGPAARAGVRVGDKLLEVNGVALQGAEHHEAVEALRGAGTAVQMRVWRE

SEQ ID NO: 25 - protein, 92 aa, Homo Sapiens
RQRHVACLARSERGLGFSIAGGKGSTPYRAGDAGIFVSRIAEGGAAHRAGTLQVGDRVLSINGVDVTEARHDHAVSLLTAASPTIALLLERE

SEQ ID NO: 26 - protein, 92 aa, Homo Sapiens
YPVEEIRLPRAGGPLGLSIVGGSDHSPFGVQEPGVFISKVLGLAARSPRGLAARGDRILAVNGQDVRDATHQEAVSALLRPCLELSLLVRRD

SEQ ID NO: 27 - protein, 92 aa, Homo Sapiens
PGLRELCIQKPGERLGISIRGGARGHAGNPRDPGIFISKVSPTGAAGRDGRLRVGLRLLEVNQQSLLGLTHGEAVQLLRSVGDTLTVLVCDG

SEQ ID NO: 28 - protein, 99 aa, Artificial Sequence
  VARIANT 1, 2, 3, 4, 5, 6, 7, 9, 12, 13, 14, 24, 25, 26, 27, 28,29, 30, 31, 32, 33, 34, 35, 36, 37, 38, 39, 40, 41, 45, 46, 48,49, 52, 54, 55, 58, 60, 61, 64, 67, 70, 71, 72, 73, 74, 75, 76,77, 79, 80, 81, 83, 84, 86, 87, 88, 89, 90, 91: Xaa = Any Amino Acid
  VARIANT 93, 94, 95, 96, 97, 98, 99: Xaa = Any Amino Acid
  Consensus sequence
XXXXXXXLXKGXXXGLGFSIAGGXXXXXXXXXXXXXXXXXXIFIXXIXXGGXAXXDGXLXXGDXILXVNXXXXXXXXHXXXVXXLXXXXXXVXXXXXXX

SEQ ID NO: 29 - protein, 4 aa, Unknow
  VARIANT 3: C- terminus of peptide; Xaa = any amino acid
QTXV

SEQ ID NO: 30 - protein, 12 aa, Homo sapiens
  BINDING (0)...(0): Recognition unit
PGRQGFSSESKV

SEQ ID NO: 31 - protein, 12 aa, Homo sapiens
  BINDING (0)...(0): Recognition unit
NSDGVNEKVSCV

SEQ ID NO: 32 - protein, 12 aa, Homo sapiens
  BINDING (0)...(0): Recognition unit
RSSSFQAEVSLV

SEQ ID NO: 33 - protein, 12 aa, Homo sapiens
  BINDING (0)...(0): Recognition unit
VSFHSIKQSTAV

SEQ ID NO: 34 - protein, 12 aa, Homo sapiens
  BINDING (0)...(0): Recognition unit
KIPFRSRSPSDV

SEQ ID NO: 35 - protein, 12 aa, Homo sapiens
  BINDING (0)...(0): Recognition unit
PCRPGFASESKV

SEQ ID NO: 36 - protein, 12 aa, Human Adenovirus type2
  BINDING (0)...(0): Recognition unit
VLPTARYHPSIV

SEQ ID NO: 37 - protein, 12 aa, Human papillomavirus type18
  BINDING (0)...(0): Recognition unit
QERLQRRRETQV

SEQ ID NO: 38 - protein, 12 aa, Herpes Simplex virus type 1
  BINDING (0)...(0): Recognition unit
CLGFIPQYLSAV

SEQ ID NO: 39 - protein, 12 aa, Human Herpesvirus 4
  BINDING (0)...(0): Recognition unit
TTPPYDDAETYV

SEQ ID NO: 40 - protein, 12 aa, Human T-cell lymphotrophic virus type 1
  BINDING (0)...(0): Recognition unit
PPSEKHFRETEV

SEQ ID NO: 41 - protein, 12 aa, Human Herpesvirus 3
  BINDING (0)...(0): Recognition unit
KNENPPTPHTDV

SEQ ID NO: 42 - protein, 12 aa, Mus musculus
  BINDING (0)...(0): Recognition unit
VYEKLSSIESDV

SEQ ID NO: 43 - protein, 12 aa, Rattus norvegicus
  BINDING (0)...(0): Recognition unit
LNLSDPSVSTVV

SEQ ID NO: 44 - protein, 12 aa, Homo sapiens
  BINDING (0)...(0): Recognition unit
ILRDYKQSSSTL

SEQ ID NO: 45 - protein, 12 aa, Homo sapiens
  BINDING (0)...(0): Recognition unit
IIRDYTQSSSSL

SEQ ID NO: 46 - protein, 12 aa, Homo sapiens
  BINDING (0)...(0): Recognition unit
PTSQCPSAHVQL

SEQ ID NO: 47 - protein, 12 aa, Homo sapiens
  BINDING (0)...(0): Recognition unit
GREVVDSTTSSL

SEQ ID NO: 48 - protein, 12 aa, Homo sapiens
  BINDING (0)...(0): Recognition unit
GYNVYGIESVKI

SEQ ID NO: 49 - protein, 12 aa, Homo sapiens
  BINDING (0)...(0): Recognition unit
NCSNAKAVETDV

SEQ ID NO: 50 - protein, 12 aa, Homo sapiens
  BINDING (0)...(0): Recognition unit
LEQRPYRRESEI

SEQ ID NO: 51 - protein, 12 aa, Homo sapiens
  BINDING (0)...(0): Recognition unit
PPSPDRDRESIV

SEQ ID NO: 52 - protein, 12 aa, Homo sapiens
  BINDING (0)...(0): Recognition unit
KPKFSISPDSLS

SEQ ID NO: 53 - protein, 12 aa, Rattus norvegicus
  BINDING (0)...(0): Recognition unit
ATYHKQVSLSHV

SEQ ID NO: 54 - protein, 12 aa, Rattus norvegiecus
  BINDING (0)...(0): Recognition unit
  VARIANT 1: Xaa = Any Amino Acid
XRLTNSQGETTV

SEQ ID NO: 55 - protein, 12 aa, Mus musculus
  BINDING (0)...(0): Recognition unit
RKEQGAGSHSDA

SEQ ID NO: 56 - protein, 12 aa, Homo sapiens
  BINDING (0)...(0): Recognition unit
ELFHPLGADSQV

SEQ ID NO: 57 - protein, 12 aa, Homo sapiens
  BINDING (0)...(0): Recognition unit
ETEKPIDSETKM

SEQ ID NO: 58 - protein, 12 aa, Homo sapiens
  BINDING (0)...(0): Recognition unit
NSNFRNEIQSLV

SEQ ID NO: 59 - protein, 12 aa, Homo sapiens
  BINDING (0)...(0): Recognition unit
SLCSESTATSPV

SEQ ID NO: 60 - protein, 12 aa, Homo sapiens
  BINDING (0)...(0): Recognition unit
GPNDSFTEATNV

SEQ ID NO: 61 - protein, 12 aa, Homo sapiens
  BINDING (0)...(0): Recognition unit
RTLLNGDLQTSI

SEQ ID NO: 62 - protein, 12 aa, Homo sapiens
  BINDING (0)...(0): Recognition unit
EETEEEVQDTRL

SEQ ID NO: 63 - protein, 12 aa, Homo sapiens
  BINDING (0)...(0): Recognition unit
SYSLVEAQKSKV

SEQ ID NO: 64 - protein, 12 aa, Homo sapiens
  BINDING (0)...(0): Recognition unit
VPEEESHRESEV

SEQ ID NO: 65 - protein, 12 aa, Homo sapiens
  BINDING (0)...(0): Recognition unit
ARELLKELRTQV

SEQ ID NO: 66 - protein, 12 aa, Homo sapiens
  BINDING (0)...(0): Recognition unit
SDSSLQSLETSV

SEQ ID NO: 67 - protein, 12 aa, Homo sapiens
  BINDING (0)...(0): Recognition unit
KRHSGSYLVTSV

SEQ ID NO: 68 - protein, 12 aa, Homo sapiens
  BINDING (0)...(0): Recognition unit
SKRQSILFSTEV

SEQ ID NO: 69 - protein, 12 aa, Homo sapiens
  BINDING (0)...(0): Recognition unit
LNQPGTPTRTAV

SEQ ID NO: 70 - protein, 12 aa, Homo sapiens
  BINDING (0)...(0): Recognition unit
ENSRPHTNETSL

SEQ ID NO: 71 - protein, 12 aa, Homo sapiens
  BINDING (0)...(0): Recognition unit
TKLDKESFLTWL

SEQ ID NO: 72 - protein, 12 aa, Homo sapiens
  BINDING (0)...(0): Recognition unit
SESTKRKLASAV

SEQ ID NO: 73 - protein, 12 aa, Bos taurus
  BINDING (0)...(0): Recognition unit
KKQKNKDKEYYV

SEQ ID NO: 74 - protein, 12 aa, Bos taurus
  BINDING (0)...(0): Recognition unit
KAKKNKDKEYYV

SEQ ID NO: 75 - protein, 15 aa, Drosophila melanogaster
  BINDING (0)...(0): Recognition unit
SRLKPAPPPPAASAG

SEQ ID NO: 76 - protein, 1111 aa, Homo sapiens
  VARIANT 23,37,58,975,977,1041: Xaa = any amino acid
  VARIANT 1056,1059,1065,1087,1100: Xaa = any amino acid
SHLPSEVISSAELPSVLPDSAGXGSEYLLEQSSLACXAECVMLQNVSKESFERTINIXKGNSSLGMTVSANKDGLGMIVRSIIHGGAISRDGRIAIGDCILSINEESTISVTNAQARAMLRRHSLIGPDIKITYVPAEHLEEFKISLGQQSGRVMALDIFSSYTGRDIPELPEREEGEGEESELQNTAYSNWNQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSIINRPRAPSQSESEPEKAPLCSVPPPPPSAFAEMGSDHTQSSASKISQDVDKEDEFGYSWKNIRERYGTLTGELHMIELEKGHSGLGLSLAGNKDRSRMSVFIVGIDPNGAAGKDGRLQIADELLEINGQILYGRSHQNASSIIKCAPSKVKIIFIRNKDAVNQMAVCPGNAVEPLPSNSDSLQNKETEPTVTTSDAAVDLSSFKNVQHLELPKDQGGLGIAISEEDTLSGVIIKSLTEHGVAATDGRLKVGDQILAVDDEIVVGYPIEKFISLLKTAKMTVKLTIHAENPDSQAVPSAAGAASGEKKNSSQSLMVPQSGSPEPESIRNTSRSSTPAIFASDPATCPIIPGCETTIEISKGRTGLGLSIVGGSDTLLGAIIIHEVYEEGAACKDGRLWAGDQILEVNGIDLRKATHDEAINVLRQTPQRVRLTLYRDEAPYKEEEVCDTLTIELQKKPGKGLGLSIVGKRNDTGVFVSDIVKGGIADADGRLMQGDQILMVNGEDVRNATQEAVAALLKCSLGTVTLEVGRIKAGPFHSERRPSQSSQVSEGSLSSFTFPLSGSSTSESLESSSKKNALASEIQGLRTVEMKKGPTDSLGISIAGGVGSPLGDVPIFIAMMHPTGVAAQTQKLRVGDRIVTICGTSTEGMTHTQAVNLLKNASGSIEMQVVAGGDVSVVTGHQQEPAXSXLSFTGLTSSSIFQDDLGPPQCKSITLERGPDGLGFSIVGGYGSPHGDLPIYVKTVFAKGAASEXGRLKRGDQIIAVNGXSLXGVTHEXAVAILKRTKGTVTLMVLSIGCXNTNPTPSSPPTVXRMHWSQFLCCV

SEQ ID NO: 77 - DNA, 2223 bp, Homo sapiens
  variation 2204: n = a, c, g, or t
gtcgacccacgcgtccgccctcggtgcctaaacaagcagcagaagccactggaagagtgtgaagatgatgaactgcctgctggttgggaaaagattgaagaccctgtctatggtatctactatgtagaccacatcaacaggaagacacaatatgagaacccggttctagaagccaaacggaagaagcagcttgagcagcagcagcagcagcaacagcagcagcaacagcagcagcagcagcagcagcagcagcagcagacagaagaatggacagaagatcactcagcccttgtgcctcctgttattccaaaccaccctccaagcaatccagagccagccagagaagttccacttcagggcaaacccttttttacacgaaacccttctgagttgaaaggcaagttcattcacacaaagctgcggaaaagcagtcgtggctttggcttcacggtggttggaggggatgaacctgatgagtttctccagatcaagagcttggtcctagatggtcctgctgcattggatggcaagatggaaacaggggatgtgattgtaagtgtgaatgacacctgtgttttgggacacacacatgctcaagttgtgaagatcttccagtccatccccattggtgccagcgttggacctgaactctgccgaggttatccattgccttttgatccagatgaccccaatacaagtttagtgacctcggtagccattttggataaagaaccaattattgtgaatgggcaagagacctatgattcaccagctagccacagtagtaaaacaggcaaagtcaatggcatgaaggatgccaggccaagcagcccagcggacgtggcttcaaatagttctcatggttatcctaatgacactgtttctttggcttcctccatagccactcagccagaactcataactgttcatattgtcaaagggccaatgggctttggttttactatcgcagacagtcctggtgggggtggccaaagagtgaaacagattgttgacagcccaaggtgccgaggcctgaaagaaggggatctcatagtggaagttaataagaagaacgtgcaggccctaactcacaaccaagtggtggatatgctggttgagtgtcctaagggaagtgaggtcacattgttggtgcaacgaggagggctgccagttcccaagaagagcccaaagtcgcaaccactggaaaggaaagacagccagaatagttctcagcacagtgtttccagccaccgaagcctgcacacagcatccccaagccacagcacacaggtgctccccgagttcccacctgcagaggcccaagctccagatcaaactgacagctctggccagaaaaaaccagatccttttaaaatctgggcccagtccaggagcatgtatgaaaaccgacttccagattaccaggaacaggacatcttcctctggagaaaagagactggatttggatttaggattctgggtggaaatgaaccaggggaacctatttatattggtcacatcgtaccactgggtgctgctgatactgacggccgcctgaggtctggagatgaattaatctgtgtggatgggacgccagtaattggaaaatcacaccagcttgtggtccagcttatgcaacaagctgccaagcaaggccacgtcaatctcacggtgcggcgtaaagtggtttttgcggtgcccaaaaccgagaacgaggtgccttcgccagcctcctctcatcacagtagcaaccagccggcttcgctgacagaagagaagcgcactccgcagggcagccagaactcgctgaacacggtgagctcgggcagcggcagcaccagcggcatcggcagtggcggcggcgggggcagcggcgtggtcagcaccgtggtgcagccctacgacgtggagatccggcgcggggagaacgagggcttcggcttcgtcatcgtgtcctcggtgagcaggcccgaagcaggcacgacctttggcaatgcatgtgtggctatgcctcacaaaataggtcggattattgaggggagccctgctgaccgctgtggcaagctgaaagtaggagaccggatcttggcagtaaatggatgttccatcaccaacaaatcccattcagacattgtgaacctaatcaaggaagcgggaaacacagttaccctccgcatcatttcctggggatgagtcctcgaatnccaccttgctgacccggaa

SEQ ID NO: 78 - protein, 740 aa, Homo sapiens
  VARIANT 734: Xaa = any amino acid
VDPRVRPRCLNKQQKPLEECEDDELPAGWEKIEDPVYGIYYVDHINRKTQYENPVLEAKRKKQLEQQQQQQQQQQQQQQQQQQQQQTEEWTEDHSALVPPVIPNHPPSNPEPAREVPLQGKPFFTRNPSELKGKFIHTKLRKSSRGFGFTVVGGDEPDEFLQIKSLVLDGPAALDGKMETGDVIVSVNDTCVLGHTHAQVVKIFQSIPIGASVGPELCRGYPLPFDPDDPNTSLVTSVAILDKEPIIVNGQETYDSPASHSSKTGKVNGMKDARPSSPADVASNSSHGYPNDTVSLASSIATQPELITVHIVKGPMGFGFTIADSPGGGGQRVKQIVDSPRCRGLKEGDLIVEVNKKNVQALTHNQVVDMLVECPKGSEVTLLVQRGGLPVPKKSPKSQPLERKDSQNSSQHSVSSHRSLHTASPSHSTQVLPEFPPAEAQAPDQTDSSGQKKPDPFKIWAQSRSMYENRLPDYQEQDIFLWRKETGFGFRILGGNEPGEPIYIGHIVPLGAADTDGRLRSGDELICVDGTPVIGKSHQLVVQLMQQAAKQGHVNLTVRRKVVFAVPKTENEVPSPASSHHSSNQPASLTEEKRTPQGSQNSLNTVSSGSGSTSGIGSGGGGGSGVVSTVVQPYDVEIRRGENEGFGFVIVSSVSRPEAGTTFGNACVAMPHKIGRIIEGSPADRCGKLKVGDRILAVNGCSITNKSHSDIVNLIKEAGNTVTLRIISWGVLEXHLADPE

SEQ ID NO: 79 - DNA, 873 bp, Homo sapiens
ccacgcgtccgattagcaccagcttcatcataccattcaacagatgcagacttcacaggctatggtggtttccaggctcctctgtcagtggaccccgcaacgtgtcccattgtccctggacaggaaatgattatagaaatatccaagggacgttcagggcttggtctcagcattgtgggaggaaaagacacacccttgaatgctatagttatccatgaagtctatgaagaaggggcagcagccagagatggaagactttgggctggtgaccagatattagaggttaatggggttgacctgaggaactccagccacgaagaagccatcacagccctgaggcagaccccccagaaggtgcggctggtggtgtatagagatgaagcacactaccgggatgaggagaacttggagattttccctgtggatctgcagaagaaagctggccggggcctgggcctgagcattgttgggaaacgaaatggaagcggagtgtttatttctgacatcgtgaaaggcggagccgcagacctggatgggagattgattcagggagatcagatcttatctgtgaatggggaggacatgagaaatgcctcacaggagacagtggccaccatcctcaagtgtgcacagggacttgtgcagctagagattggaagactccgagctggttcctggacctccgcaaggacgacatcacagaacagtcagggtagtcagcagagtgcacacagcagctgtcatccctccttcgctcctgtcatcactggcctgcaaaacctggttggcacaaaaagagtttcagatccttcccagaaaaattcaggcacagatatggaaccaaggactgttgagataaacagggagctcagt

SEQ ID NO: 80 - protein, 354 aa, Homo sapiens
VDPRVRPRCLNKQQKPLEECEDDELPAGWEKIEDPVYGIYYVDHINRKTQYENPVLEAKRKKQPRVRLAPASSYHSTDADFTGYGGFQAPLSVDPATCPIVPGQEMIIEISKGRSGLGLSIVGGKDTPLNAIVIHEVYEEGAAARDGRLWAGDQILEVNGVDLRNSSHEEAITALRQTPQKVRLVVYRDEAHYRDEENLEIFPVDLQKKAGRGLGLSIVGKRNGSGVFISDIVKGGAADLDGRLIQGDQILSVNGEDMRNASQETVATILKCAQGLVQLEIGRLRAGSWTSARTTSQNSQGSQQSAHSSCHPSFAPVITGLQNLVGTKRVSDPSQKNSGTDMEPRTVEINRELS

SEQ ID NO: 81 - protein, 4 aa, Artificial Sequence
  expanded PDZ consensus motif
  VARIANT 1,3: Xaa = any amino acid
  VARIANT 2: Xaa = Ser or Thr
  VARIANT 4: Xaa = small hydrophobic C-terminal amino acid
XXXX

SEQ ID NO: 82 - DNA, 260 bp, Homo Sapiens
gaggaagctgcagctgagaagcggggcctacagcgccgggccacacctcaccccagcgagctcaaggtgatgaagaggagcatcgaggggcggcggagcgaggcctgcccttgccagccagactctgggtcgcccttgcctgcagaggaggagaagcggctgagtgccgagtctggcctgagtgaagactctcgcccatctgccagcacagtctctgaggctgagcccgagggcccgtcggctgaggcacagggtgggag

SEQ ID NO: 83 - DNA, 276 bp, Homo Sapiens
cgtcagcgccacgtggcctgcctggcacgcagcgagagggggctgggcttcagcattgctggtgggaaaggctccacaccctacagggctggtgatgcgggcatcttcgtctcccgcattgccgagggcggtgctgctcaccgcgcgggcacactgcaggttggcgaccgcgtcctctctattaatggagtggacgtgactgaggccaggcatgaccacgccgtctccctgctgaccgctgcctcccccaccatcgccctgctgttggagcgggag

SEQ ID NO: 84 - DNA, 171 bp, Homo Sapiens
tacccagtggaggagatccgtctgccaagagctgggggccctctggggcttagtattgtcggaggctccgaccattccagccacccgtttggtgtccaggagcctggtgtgttcatctccaaggtctcccgcggggcctggccgctcgcagcggcctgcgggttggggacc

SEQ ID NO: 85 - DNA, 340 bp, Homo Sapiens
cctggcagtgaacgggcaagacgtgcgggatgccacgcaccaagaagcagtcagtgccctgctccggccctgcctggagctgtcgctgctggtgcggagggacccggcacccccgggcctacgggaactgtgcatccagaaggcacctggggagaggctgggcatcagcatccccgggggtgccaggggccacgctggcaacccccgcgaccccacagacgagggcatcttcatctccaaggtgagccccacgggggcagccgggcgcgacggtcggctgcgtgtgcgtttgcggctgttggaggtgaaccagcagagcctgctgggcctgacgcacggc

SEQ ID NO: 86 - protein, 16 aa, Unknow
SGSGPPSPDRDRESIV

SEQ ID NO: 87 - protein, 10 aa, Artificial Sequence
  formula representing the soluble mixture of peptides used for oriented peptide library approach
  VARIANT (3)...(10): Xaa = any amino acid except Cys and Trp
KNXXXXXXXX

SEQ ID NO: 88 - protein, 10 aa, Artificial Sequence
  formula representing the soluble mixture of peptides used for oriented peptide library approach
  VARIANT 3,4,5,6,7,8,10: Xaa = any amino acid except Cys and Trp
  VARIANT 9: Xaa = Ser or Thr or Tyr
KNXXXXXXXX

SEQ ID NO: 89 - protein, 4 aa, Artificial Sequence
  SGSG  linker
SGSG

SEQ ID NO: 90 - protein, 4 aa, Artificial Sequence
  KGKG  linker
KGKG

SEQ ID NO: 91 - protein, 4 aa, Homo Sapiens
GYGF

SEQ ID NO: 92 - protein, 4 aa, Artificial Sequence
  extended PDZ C-terminal consensus sequence
  VARIANT 1,3: Xaa = Any amino acid
  VARIANT 2: Xaa = Ser or Thr
XXXL

SEQ ID NO: 93 - protein, 4 aa, Homo Sapiens
GLGL

SEQ ID NO: 94 - protein, 4 aa, Homo Sapiens
GLGI

SEQ ID NO: 95 - protein, 4 aa, Homo Sapiens
GFGF

SEQ ID NO: 96 - DNA, 3343 bp, Homo Sapiens
  variation 69,111,173,2925,2931,3122,3169,3176,3194,3265,3307: n = a, c, g, or t
atctcatttaccaagtgaagttatatcaagtgcagaacttccttctgtgctacccgattcagctggaangggctctgagtacctgcttgaacagagctccctggcctgtantgctgagtgtgtcatgcttcaaaatgtatctaaagaatcttttgaaaggactattaatatancaaaaggcaattctagcctaggaatgacagttagtgctaataaagatggcttggggatgatcgttcgaagcattattcatggaggtgccattagtcgagatggccggattgccattggggactgcatcttgtccattaatgaagagtctaccatcagtgtaaccaatgcccaggcacgagctatgttgagaagacattctctcattggccctgacataaaaattacttatgtgcctgcagaacatttggaagagttcaaaataagcttgggacaacaatctggaagagtaatggcactggatattttttcttcatacactggcagagacattccagaattaccagagcgagaagagggagagggtgaagaaagcgaacttcaaaacacagcatatagcaattggaatcagcccaggcgggtggaactctggagagaaccaagcaaatccttaggcatcagcattgttggtggacgagggatggggagtcggctaagcaatggagaagtgatgaggggcattttcatcaaacatgttctggaagatagtccagctggcaaaaatggaaccttgaaacctggagatagaatcgtagaggtggatggaatggacctcagagatgcaagccatgaacaagctgtggaagccattcggaaagcaggcaaccctgtagtctttatggtacagagcattataaacagaccaagggcacccagtcagtcagagtcagagccagagaaggctccattgtgcagtgtgcccccaccccctccttcagcctttgccgaaatgggtagtgatcacacacagtcatctgcaagcaaaatctcacaagatgtggacaaagaggatgagtttggttacagctggaaaaatatcagagagcgttatggaaccctaacaggcgagctgcatatgattgaactggagaaaggtcatagtggtttgggcctaagtcttgctgggaacaaagaccgatccaggatgagtgtcttcatagtggggattgatccaaatggagctgcaggaaaagatggtcgattgcaaattgcagatgagcttctagagatcaatggtcagattttatatggaagaagtcatcagaatgcctcatcaatcattaaatgtgccccttctaaagtgaaaataatttttatcagaaataaagatgcagtgaatcagatggccgtatgtcctggaaatgcagtagaacctttgccttctaactcagacagtcttcaaaataaggagacagagccaactgttactacttctgatgcagctgtggacctcagttcatttaaaaatgtgcaacatctggagcttcccaaggatcaggggggtttgggtattgctatcagcgaagaagatacactcagtggagtcatcataaagagcttaacagagcatggggtagcagccacggatggacgactcaaagtcggagatcagatactggctgtagatgatgaaattgttgttggttaccctattgaaaagtttattagccttctgaagacagcaaagatgacagtaaaacttaccatccatgctgagaatccagattcccaggctgttccttcagcagctggtgcagccagtggagaaaaaaagaacagctcccagtctctgatggtcccacagtctggctccccagaaccggagtccatccgaaatacaagcagatcatcaacaccagcaatttttgcttctgatcctgcaacctgccccattatccctggctgcgaaacaaccatcgagatttccaaagggcgaacagggctgggcctgagcatcgttgggggttcagacacgctgctgggtgccattattatccatgaagtttatgaagaaggagcagcatgtaaagatggaagactctgggctggagatcagatcttagaggtgaatggaattgacttgagaaaggccacacatgatgaagcaatcaatgtcctgagacagacgccacagagagtgcgcctgacactctacagagatgaggccccatacaaagaggaggaagtgtgtgacaccctcactattgagctgcagaagaagccgggaaaaggcctaggattaagtattgttggtaaaagaaacgatactggagtatttgtgtcagacattgtcaaaggaggaattgcagatgccgatggaagactgatgcagggagaccagatattaatggtgaatggggaagacgttcgtaatgccacccaagaagcggttgccgctttgctaaagtgttccctaggcacagtaaccttggaagttggaagaatcaaagctggtccattccattcagagaggaggccatctcaaagcagccaggtgagtgaaggcagcctgtcatctttcacttttccactctctggatccagtacatctgagtcactggaaagtagctcaaagaagaatgcattggcatctgaaatacagggattaagaacagtcgaaatgaaaaagggccctactgactcactgggaatcagcatcgctggaggagtaggcagcccacttggtgatgtgcctatatttattgcaatgatgcacccaactggagttgcagcacagacccaaaaactcagagttggggataggattgtcaccatctgtggcacatccactgagggcatgactcacacccaagcagttaacctactgaaaaatgcatctggctccattgaaatgcaggtggttgctggaggagacgtgagtgtggtcacaggtcatcagcaggagcctgcaanttccantctttctttcactgggctgacgtcaagcagtatatttcaggatgatttaggacctcctcaatgtaagtctattacactagagcgaggaccagatggcttaggcttcagtatagttggaggatatggcagccctcatggagacttacccatttatgttaaaacagtgtttgcaaagggagcagcctctgaanacggacgtctgaaaaggggcgatcagatcattgctgtcaatgggcanagtctanaaggagtcacccatgaanaagctgttgccatccttaaacggacaaaaggcactgtcactttgatggttctctcttgaattggctgccanaattgaaccaacccaacccctagctcacctcctactgtaaanagaatgcactggtcctgacaatttttatgctgtgtt

SEQ ID NO: 97 - protein, 4 aa, Homo Sapiens
QTSV

SEQ ID NO: 98 - DNA, 2662 bp, Homo Sapiens
gaattccggaaatcagtgagcaacatggagaaagccagtatagagcctcctgaggaggaagaggaagagaggcctgtggtcaatggaaatggagtagtagtaacaccagaatccagtgaacatgaagacaaaagtgcaggtgcctcaggggagatgccctcccagccttatcctgcaccagtgtacagtcagcctgaggagctgaaggagcagatggatgacacaaagccaactaaacctgaagacaatgaggaaccagacccattgcctgataactgggaaatggcctatacagagaagggcgaagtctacttcattgaccataacacaaagacaacatcatggctggatccacgacttgcgaaaaaggctaaacctccagaagagtgcaaagaaaatgagcttccatatggctgggaaaaaatcgatgatcccatttatggcacttattatgttgaccacataaatagaaaaacacagtttgaaaatcctgtcctggaagcaaaaaggaagctacagcaacataacatgccccacacaaaacttggaacaaagcccctgcaggccccaggtttccgaaaaaaaccactcttcacccgggatgcatcccagttgaagggaacattcctcagcaccaccctaaaaaagagcaacatgggctttggatttaccatcattggtggaaacaagcctgatgagtttctgcaggtgaaaagtgtgattccggatgggcctgcagcacaggatggaaaaatggaaacaggtgatgtcattgtctatattaatgaagtttgtgtccttggacacactcatgcagatgttgtcaaacttttccagtctgttcctattggtcagagtgtcaacctggtgttgtgtcgtggctaccctttgccctttgatcctgaagaccctgctaacagcatggtgccaccccttgcaataatggagaggccacctccagtgatggtcaatggaagacacaactatgaaacatatttggagtacatttctcggacctcacagtcagttccagatataacagatcggccgcctcattctctgcactccatgccaactgatggtcagctagacggcacgtatccaccgcccgtccatgatgacaatgtgtctatggcttcatctggggccacccaagctgaacttatgaccttaaccattgtgaaaggtgcccagggcttcggcttcactattgccgacagtcctacaggacagcgggtgaaacaaatacttgacattcagggatgccctggcctgtgtgaaggcgacctcattgttgagatcaaccagcagaatgtacagaacctgagccatacagaagtagtggatatacttaaggactgtcccattggaagtgaaacttctttgattatccatcgaggaggtttcttttctccatggaaaactccaaagcctataatggaccgatgggagaatcaaggcagtcctcaaacgagtttatctgctccggccataccgcagaacctgcccttcccacctgcccttcacaggagctcctttcctgactcaacagaggcctttgacccacggaagcctgatccatatgagctctacgagaaatctagggccatttatgaaagtaggcgtccagattataaggaattggatgttcatcttcggaggatggagtctggatttggcttcagaatcctcgggggagatgagcctggacagcctattttgattggagctgtcattgccatgggctcagccgacagagatggccgccttcacccaggagatgagcttgtgtatgttgatgggattccagtagccggcaaaacccaccgctatgtcatcgacctcatgcaccacgcagcccgcaatgggcaggtcaacctcactgtgagaagaaaggtgctatgtggaggggagccctgcccagagaacgggagaagtccaggctctgtatccacccaccacagctctccacgcagtgactacgcaacctacaccaacagcaaccacgctgcccccagtagcaatgcctctccccctgaaggcttcgcctcccacagcctgcagaccagtgatgtggtcattcaccgcaaagagaatgagggcttcggctttgtcatcatcagctccctgaacaggcctgagtctggatccactataactgtgccccataaaatcggacgcatcattgatgggagtcctgcagatcgctgtgcaaaactaaaagtgggagaccggatcctagcagtgaatggccagtctatcatcaacatgcctcacgctgacatcgtgaagctcatcaaggatgcaggtcttagtgtcacccttcgcatcattcctcaggaggagctcaacagccccacctcggcacccagctcagagaagcagagtcccatggcgcagcagagtcccctggcacagcagagtcccctggcccagccaagcccagccacccccaacagccccatcgcccagccagcaccacctcaaccacttcagctgcaaggacacgaaaatagttacaggtcggaagtgaaagcaaggcaagatgtgaaaccagacatccgacagcctccattcacagactacaggcagcccccgctggattacaggcaacccccaggaggggaataccagcagcccggaattccagctgagcgccggt

SEQ ID NO: 99 - protein, 887 aa, Homo Sapiens
EFRKSVSNMEKASIEPPEEEEEERPVVNGNGVVVTPESSEHEDKSAGASGEMPSQPYPAPVYSQPEELKEQMDDTKPTKPEDNEEPDPLPDNWEMAYTEKGEVYFIDHNTKTTSWLDPRLAKKAKPPEECKENELPYGWEKIDDPIYGTYYVDHINRKTQFENPVLEAKRKLQQHNMPHTKLGTKPLQAPGFRKKPLFTRDASQLKGTFLSTTLKKSNMGFGFTIIGGDEPDEFLQVKSVIPDGPAAQDGKMETGDVIVYINEVCVLGHTHADVVKLFQSVPIGQSVNLVLCRGYPLPFDPEDPANSMVPPLAIMERPPPVMVNGRHNYETYLEYISRTSQSVPDITDRPPHSLHSMPTDGQLDGTYPPPVHDDNVSMASSGATQAELMTLTIVKGAQGFGFTIADSPTGQRVKQILDIQGCPGLCEGDLIVEINQQNVQNLSHTEVVDILKDCPIGSETSLIIHRGGFFSPWKTPKPIMDRWENQGSPQTSLSAPAIPQNLPFPPALHRSSFPDSTEAFDPRKPDPYELYEKSRAIYESRRPDYKELDVHLRRMESGFGFRILGGDEPGQPILIGAVIAMGSADRDGRLHPGDELVYVDGIPVAGKTHRYVIDLMHHAARNGQVNLTVRRKVLCGGEPCPENGRSPGSVSTHHSSPRSDYATYTNSNHAAPSSNASPPEGFASHSLQTSDVVIHRKENEGFGFVIISSLNRPESGSTITVPHKIGRIIDGSPADRCAKLKVGDRILAVNGQSIINMPHADIVKLIKDAGLSVTLRIIPQEELNSPTSAPSSEKQSPMAQQSPLAQQSPLAQPSPATPNSPIAQPAPPQPLQLQGHENSYRSEVKARQDVKPDIRQPPFTDYRQPPLDYRQPPGGEYQQPGIPAERR

SEQ ID NO: 100 - DNA, 2521 bp, Homo Sapiens
cgtcgacatttcatttccgttgaacagttcaaagcactggaagagagtggagcattgttagaaagtgggacatatgatggaaacttctatcgaactcccaatcctccagcagaacccagcccttttcagccagatccagttgatcaagtcctctttgataatgagtttgatgcagaatctcaaagaaaacgaacgacatctgtcagcaagatggaaagaatggatagctctcttcctgaagaggaagaagatgaggacaaggaagctattaatggcagtggaaacgcagaaaacagagagaggcattctgagtcatctgactggatgaagactgttccaagttacaaccaaacaaatagctccatggactttagaaattatatgatgagagatgagactctggaaccactgcccaaaaactgggaaatggcctacactgacacagggatgatctacttcattgaccacaataccaagacaaccacctggttggatcctcgtctttgtaagaaagccaaagcccctgaagactgtgaagatggagagcttccttatggctgggagaaaatagaggaccctcagtatgggacatactatgttgatcaccttaaccagaaaacccagtttgaaaatccagtggaggaagccaaaaggaaaaagcagttaggacaggttgaaattgggtcttcaaaaccagatatggaaaaatcacacttcacaagagatccatcccagcttaaaggtgtccttgttcgagcatcactgaaaaaaagcacaatgggatttggttttactattattggtggagatagacctgatgagttcctacaagtgaaaaatgtgctgaaagatggtcccgcagctcaggatgggaaaattgcaccaggcgatgttattgtagacatcaatggcaactgtgtcctcggtcacactcatgcagatgttgtccagatgtttcaattggtacctgtcaatcagtatgtaaacctcactttatgtcgtggttatccacttcctgatgacagtgaagatcctgttgtggacattgttgctgctacccctgtcatcaatggacagtcattaaccaagggagagacttgcatgaatcctcaggattttaagccaggagcaatggttctggagcagaatggaaaatcgggacacactttgactggtgatggtctcaatggaccatcagatgcaagtgagcagagagtatccatggcatcgtcaggcagctcccagcctgaactagtgactatccctttgattaagggccctaaagggtttgggtttgcaattgctgacagccctactggacagaaggtgaaaatgatactggatagtcagtggtgtcaaggccttcagaaaggagatataattaaggaaatataccatcaaaatgtgcagaatttaacacatctccaagtggtacaggtgctaaagcagtttccagtaggtgctgatgtaccattgcttatcttaagaggaggtcctccttcaccaaccaaaactgccaaaatgaaaacagataaaaaggaaaatgcaggaagtttggaggccataaatgagcctattcctcagcctatgccttttccaccgagcattatcaggtcaggatccccaaaattggatccttctgaggtctacctgaaatctaagactttatatgaagataaaccaccaaacaccaaagatttggatgtttttcttcgaaaacaagagtcagggtttggcttcagggtgctaggaggagatcgacctgaccagtctatatatattggggctattattcccctgggagcagctgagaaagatggtcggctccgcccagctgatgaactaatgtgcattgatggaattcctgttaaagggaaatcacacaaacaagtcttggacctcatgacaactgctgctcgaaatggccatgtgttactaactgtcagacggaagatcttctatggagaaaaacaacccgaggacgacagctctcaggccttcatttcaacacagaatggatctccccgcctgaaccgggcagaggtcccagccaggcctgcaccccaggagccctatgatgttgtcttgcaacgaaaagaaaatgaaggatttggctttgtcatcctcacctccaaaaacaaaccacctccaggagttattcctcataaaattggccgagtcatagaaggaagtccggctgaccgctgtggaaaactgaaagttggagatcatatctctgcagtgaatgggcagtccattgttgaactgtctcatgataacattgttcagctgatcaaagatgctggtgtcaccgtcacactaacggtcattgctgaagaagagcatcatggtccaccatcaggaacaaactcagccaggcaaagcccagccctgcagcacaggcccatgggacagtcacaggccaaccacatacctggggacagaagtgccctagaaggtgaaattggaaagatgtctccacttcttacagacattcttggtcagaccacaagcaccttgt

SEQ ID NO: 101 - protein, 840 aa, Homo Sapiens
RRHFISVEQFKALEESGALLESGTYDGNFYRTPNPPAEPSPFQPDPVDQVLFDNEFDAESQRKRTTSVSKMERMDSSLPEEEEDEDKEAINGSGNAENRERHSESSDWMKTVPSYNQTNSSMDFRNYMMRDETLEPLPKNWEMAYTDTGMIYFIDHNTKTTTWLDPRLCKKAKAPEDCEDGELPYGWEKIEDPQYGTYYVDHLNQKTQFENPVEEAKRKKQLGQVEIGSSKPDMEKSHFTRDPSQLKGVLVRASLKKSTMGFGFTIIGGDRPDEFLQVKNVLKDGPAAQDGKIAPGDVIVDINGNCVLGHTHADVVQMFQLVPVNQYVNLTLCRGYPLPDDSEDPVVDIVAATPVINGQSLTKGETCMNPQDFKPGAMVLEQNGKSGHTLTGDGLNGPSDASEQRVSMASSGSSQPELVTIPLIKGPKGFGFAIADSPTGQKVKMILDSQWCQGLQKGDIIKEIYHQNVQNLTHLQVVQVLKQFPVGADVPLLILRRGPPSPTKTAKMKTDKKENAGSLEAINEPIPQPMPFPPSIIRSGSPKLDPSEVYLKSKTLYEDKPPNTKDLDVFLRKQESGFGFRVLGGDGPDQSIYIGAIIPLGAAEKDGRLRAADELMCIDGIPVKGKSHKQVLDLMTTAARNGHVLLTVRRKIFYGEKQPEDDSSQAFISTQNGSPRLNRAEVPARPAPQEPYDVVLQRKENEGFGFVILTSKNKPPPGVIPHKIGRVIEGSPADRCGKLKVGDHISAVNGQSIVELSHDNIVQLIKDAGVTVTLTVIAEEEHHGPPSGTNSARQSPALQHRPMGQSQANHIPGDRSALEGEIGKDVSTSYRHSWSDHKHL

SEQ ID NO: 102 - DNA, 4607 bp, Homo Sapiens
atgcagctggtggagctggacgtgtcccggaacgatatccctgagatcccggagagcatcaagttctgcaaggctctggagatcgcggacttcagcgggaaccccctctccaggctccctgatggcttcactcagctgcgcagcctggctcacctggccctgaatgatgtgtctctgcaggcactgcccggggacgtgggcaacctcgccaacctggtgaccctggagctccgggagaacctgctcaagtccctgccagcgtccctgtcatttctggtcaagctggaacagctggatctgggaggcaacgatctggaagtgctgccagacactctgggggctctgcccaatcttcgggagctgtggcttgaccggaaccagctgtcagcactgcccccggagctcgggaacctgcggcgcctggtgtgcctggacgtgtcggaaaaccggctggaggagctgcctgctgagctcggcgggctggtgctgctcactgacctgctgctgtcccagaacctgctgcggaggctgcccgacggcatcggtcagctgaagcagctatccatcctaaaggtagaccagaatcggctgtgcgaggtgactgaggccatcggggactgtgagaacctctctgagctgatcctcacggagaacctgctgatggccctgccccgctccctgggaaagctgactaagctgaccaacctcaacgtggaccggaaccacctcgaggcgctgccgcccgagatcgggggctgtgtggcactcagcgtcctctccttgagggacaaccgcctggccgtcctgccaccagagctggcccacacgacagagctgcacgtgctggacgtggcggggaaccgcctgcagagtctgccgttcgcgctcacccacctcaatctcaaggccctgtggctggcagagaaccaggcgcagcccatgctccggttccagacggaggatgatgcccggaccggcgagaaggtgctcacctgctacttgctgccccagcagcccccactcagcctcgaggatgctgggcagcaggggagcctctcggagacctggagcgatgccccgccgagccgcgtcagcgtcatccagttcctggaggcccccataggtgatgaggacgctgaggaagctgcagctgagaagcggggcctacagcgccgggccacacctcaccccagcgagctcaaggtgatgaagaggagcatcgaggggcggcggagcgaggcctgcccttgccagccagactctgggtcgcccttgcctgcagaggaggagaagcggctgagtgccgagtctggcctgagtgaagactctcgcccatctgccagcacagtctctgaggctgagcccgagggcccgtcggctgaggcacagggtgggagccagcaggaagccacgactgctggcggggaggaagacgccgaagaggactaccaggagcccacggtgcatttcgcagaggacgcactgctgcccggggatgacagggagatcgaggaggggcagcctgaggccccctggaccctgccaggcgggaggcagcggctcatccgcaaggacacacctcactacaaaaagcacttcaagatctccaagctgccccagcccgaggccgttgtggctctgctgcagggcatgcagcctgatggggagggccctgtggctcccgggggctggcacaatggcccccacgcaccctgggctcctcgggcccagaaggaggaggaggaggaggaagagggtagtcctcaggaggaggaggaagaggaggaggaggaaaacagggctgaagaggaagaggccagcactgaggaggaggacaaggagggggccgtggtttctgcgccctctgtcaagggagtttcgtttgaccaggccaataacctgctgatagagcctgctcgcattgaggaggaagagctgaccctcaccatcctgcggcagactgggggcctgggcatcagcattgcgggcggcaagggctccacaccctataagggggacgacgagggcatattcatctctcgggtgtccgaggaaggccctgcggcccgggctggagtccgtgtgggtgacaagctcctggaggtgaatggtgtggctctgcagggcgccgagcaccacgaggccgtggaggcgctccggggggccggcactgccgtgcagatgcgagtgtggcgggagcgcatggtggagcctgagaacgcggtcaccatcacgccgctgcggcccgaggatgattacagcccccgagagcggcggggaggggggctgcgcctgcccctgctcccgcctgaaagccccgggcccctccgtcagcgccacgtggcctgcctggcacgcagcgagagggggctgggcttcagcattgctggtgggaaaggctccacaccctacagggctggtgatgcgggcatcttcgtctcccgcattgccgagggcggtgctgctcaccgcgcgggcacactgcaggttggcgaccgcgtcctctctattaatggagtggacgtgactgaggccaggcatgaccacgccgtctccctgctgaccgctgcctcccccaccatcgccctgctgttggagcgggaggctgggggccctcttcctcccagccctctgccacattcctcaccccccaccgctgctgttgccaccaccagcataaccactgccacccccggggtgcctgggttgccgagcctggcccccagcctgctggctgccgcgttggaagggccatacccagtggaggagatccgtctgccaagagctgggggccctctggggcttagtattgtcggaggctccgaccattccagccacccgtttggtgtccaggagcctggtgtgttcatctccaaggtctcccgcggggcctggccgctcgcagcggcctgcgggttggggaccgcatcctggcagtgaacgggcaagacgtgcgggatgccacgcaccaagaagcagtcagtgccctgctccggccctgcctggagctgtcgctgctggtgcggagggacccggcacccccgggcctacgggaactgtgcatccagaaggcacctggggagaggctgggcatcagcatccccgggggtgccaggggccacgctggcaacccccgcgaccccacagacgagggcatcttcatctccaaggtgagccccacgggggcagccgggcgcgacggtcggctgcgtgtgcgtttgcggctgttggaggtgaaccagcagagcctgctgggcctgacgcacggcgaggcggtgcagctgctccgcagtgtgggcgacaccctcaccgtgctggtctgtgacggcttcgaggccagcaccgacgcagccctggaggtgtccccaggtgtcattgccaacccctttgcggcaggcatcggccaccggaacagcctggagagcatctcttccatcgaccgggagctgagccctgagggcccaggcaaggagaaggagctgcctggacagaccctgcactgggggcccgaggccacagaagccgcaggtcggggtctgcagcccctgaagctggactaccgcgccctggccgccgtgcccagcgctggcagcgtgcagagggtaccgtctggagcagctggagggaagatggctgaatctccctgctcccctagtggccagcagccgccctccccgccttctccggatgagctgcccgccaatgtgaagcaggcctacagggccttcgcggccgtgcccacttctcacccgcctgaggatgcccctgcccagccccccacgcctgggcctgcagcctccccggagcagctgtccttccgggagcggcagaagtactttgagctggaggtgcgcgtgccccaggccgagggcccccctaagcgcgtgtccctggtgggtgctgacgacctgcggaagatgcaggaggaggaagccagaaaactacagcagaagagagcgcagatgctgcgggaggcggcagaggctggggccgaagcgaggctcgccctggacggggagacgctgggcgaggaggaacaggaggatgagcagccaccctgggccagcccgagccccacctcaaggcagagcccggcgtcccccccgcccctgggaggtggcgccccggtgcggacggccaaagctgaacggcgccaccaggagcggctgcgcgtgcagagtccggagccaccggcacccgagcgtgccctgtcccctgccgagctccgggccctggaggccgagaagcgtgcgctgtggagggcagccaggatgaagtcattggaacaggacgctctccgagcacagatggtcctcagcaggtcccaggaaggccggggcacgcgggggcccctggagcgactggccgaggccccttcccctgcgcccaccccgtcgcccacccctgtggaagaccttggcccccagaccagcacctccccgggacgcctgtcaccggactttgctgaggagttgaggtccctggaaccatctcccagccctggcccgcagaggaggatggagaagtggctctggtgcttctgggcaggccctcacccggcgctgtgggccctgaagatgtggcactgtgcagcagccgccgccccg

SEQ ID NO: 103 - protein, 1535 aa, Homo Sapiens
MQLVELDVSRNDIPEIPESIKFCKALEIADFSGNPLSRLPDGFTQLRSLAHLALNDVSLQALPGDVGNLANLVTLELRENLLKSLPASLSFLVKLEQLDLGGNDLEVLPDTLGALPNLRELWLDRNQLSALPPELGNLRRLVCLDVSENRLEELPAELGGLVLLTDLLLSQNLLRRLPDGIGQLKQLSILKVDQNRLCEVTEAIGDCENLSELILTENLLMALPRSLGKLTKLTNLNVDRNHLEALPPEIGGCVALSVLSLRDNRLAVLPPELAHTTELHVLDVAGNRLQSLPFALTHLNLKALWLAENQAQPMLRFQTEDDARTGEKVLTCYLLPQQPPLSLEDAGQQGSLSETWSDAPPSRVSVIQFLEAPIGDEDAEEAAAEKRGLQRRATPHPSELKVMKRSIEGRRSEACPCQPDSGSPLPAEEEKRLSAESGLSEDSRPSASTVSEAEPEGPSAEAQGGSQQEATTAGGEEDAEEDYQEPTVHFAEDALLPGDDREIEEGQPEAPWTLPGGRQRLIRKDTPHYKKHFKISKLPQPEAVVALLQGMQPDGEGPVAPGGWHNGPHAPWAPRAQKEEEEEEEGSPQEEEEEEEEENRAEEEEASTEEEDKEGAVVSAPSVKGVSFDQANNLLIEPARIEEEELTLTILRQTGGLGISIAGGKGSTPYKGDDEGIFISRVSEEGPAARAGVRVGDKLLEVNGVALQGAEHHEAVEALRGAGTAVQMRVWRERMVEPENAVTITPLRPEDDYSPRERRGGGLRLPLLPPESPGPLRQRHVACLARSERGLGFSIAGGKGSTPYRAGDAGIFVSRIAEGGAAHRAGTLQVGDRVLSINGVDVTEARHDHAVSLLTAASPTIALLLEREAGGPLPPSPLPHSSPPTAAVATTSITTATPGVPGLPSLAPSLLAAALEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAPPGLRELCIQKAPGERLGISIRGGARGHAGNPRDPTDEGIFISKVSPTGAAGRDGRLRVGLRLLEVNQQSLLGLTHGEAVQLLRSVGDTLTVLVCDGFEASTDAALEVSPGVIANPFAAGIGHRNSLESISSIDRELSPEGPGKEKELPGQTLHWGPEATEAAGRGLQPLKLDYRALAAVPSAGSVQRVPSGAAGGKMAESPCSPSGQQPPSPPSPDELPANVKQAYRAFAAVPTSHPPEDAPAQPPTPGPAASPEQLSFRERQKYFELEVRVPQAEGPPKRVSLVGADDLRKMQEEEARKLQQKRAQMLREAAEAGAEARLALDGETLGEEEQEDEQPPWASPSPTSRQSPASPPPLGGGAPVRTAKAERHQERLRVQSPEPPAPERALSPAELRALEAEKRALWRAARMKSLEQDALRAQMVLSRSQEGRGTRGPLERLAEAPSPAPTPSPTPVEDLGPQTSTSPGRLSPDFAEELRSLEPSPSPGPQRRMEKWLWCFWAGPHPALWALKMWHCAAAAAP

SEQ ID NO: 104 - protein, 5 aa, Artificial Sequence
  C-terminal of the inhibitor
RESIV

SEQ ID NO: 105 - protein, 5 aa, Artificial Sequence
  scrambled version of C-terminal of the inhibitor
ISVRE

SEQ ID NO: 106 - protein, 91 aa, Homo Sapiens
YEFEEITLERGNSGLGFSIAGGTDNPHIGDDPGIFITKIIPGGAAAEDGRLRVNDCILRVNEVDVSEVSHSKAVEALKEAGSIARLYVRRR

SEQ ID NO: 107 - protein, 91 aa, Homo Sapiens
ETVVEIKLFKGPKGLGFSIAGGVGNQHIPGDNSIYVTKIIDGGAAQKDGRLQVGDRLLMVNNYSLEEVTHEEAVAILKNTSEVVYLKVGNP

SEQ ID NO: 108 - protein, 85 aa, Homo Sapiens
GEPRKVVLHKGSTGLGFNIVGGEDGEGIFVSFILAGGPADLSGELQRGDQILSVNGIDLRGASHEQAAAALKGAGQTVTIIAQYQ

SEQ ID NO: 109 - protein, 91 aa, Homo Sapiens
MEYEEITLERGNSGLGFSIAGGTDNPHIGDDPSIFITKIIPGGAAAQDGRLRVNDSILFVNEVDVREVTHSAAVEALKEAGSIVRLYVMRR

SEQ ID NO: 110 - protein, 91 aa, Homo Sapiens
EKVMEIKLIKGPKGLGFSIAGGVGNQHIPGDNSIYVTKIIEGGAAHKDGRLQIGDKILAVNSVGLEDVMHEDAVAALKNTYDVVYLKVAKP

SEQ ID NO: 111 - protein, 85 aa, Homo Sapiens
  VARIANT 9: Xaa = any amino acid
SFERTINIXKGNSSLGMTVSANKDGLGMIVRSIIHGGAISRDGRIAIGDCILSINEESTISVTNAQARAMLRRHSLIGPDIKITY

SEQ ID NO: 112 - protein, 96 aa, Homo Sapiens
NQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSI

SEQ ID NO: 113 - protein, 87 aa, Homo Sapiens
GCETTIEISKGRTGLGLSIVGGSDTLLGAIIIHEVYEEGAACKDGRLWAGDQILEVNGIDLRKATHDEAINVLRQTPQRVRLTLYRD

SEQ ID NO: 114 - protein, 85 aa, Homo Sapiens
DTLTIELQKKPGKGLGLSIVGKRNDTGVFVSDIVKGGIADADGRLMQGDQILMVNGEDVRNATQEAVAALLKCSLGTVTLEVGRI

SEQ ID NO: 115 - protein, 90 aa, Homo Sapiens
QGLRTVEMKKGPTDSLGISIAGGVGSPLGDVPIFIAMMHPTGVAAQTQKLRVGDRIVTICGTSTEGMTHTQAVNLLKNASGSIEMQVVAG

SEQ ID NO: 116 - protein, 88 aa, Homo Sapiens
  VARIANT 61,64,70: Xaa = any amino acid
PQCKSITLERGPDGLGFSIVGGYGSPHGDLPIYVKTVFAKGAASEXGRLKRGDQIIAVNGXSLXGVTHEXAVAILKRTKGTVTLMVLS

SEQ ID NO: 117 - protein, 86 aa, Homo Sapiens
TKDLDVFLRKQESGFGFRVLGGDGPDQSIYIGAIIPLGAAEKDGRLRAADELMCIDGIPVKGKSHKQVLDLMTTAARNGHVLLTVR

SEQ ID NO: 118 - protein, 90 aa, Homo Sapiens
EPYDVVLQRKENEGFGFVILTSKNKPPPGVIPHKIGRVIEGSPADRCGKLKVGDHISAVNGQSIVELSHDNIVQLIKDAGVTVTLTVIAE

SEQ ID NO: 119 - protein, 12 aa, Homo Sapiens
  BINDING (0)...(0): Recognition unit
RLTNSRHGETTV

SEQ ID NO: 120 - DNA, 255 bp, Homo Sapiens
  variation 24: n = a, c, g, or t
tcttttgaaaggactattaatatancaaaaggcaattctagcctaggaatgacagttagtgctaataaagatggcttggggatgatcgttcgaagcattattcatggaggtgccattagtcgagatggccggattgccattggggactgcatcttgtccattaatgaagagtctaccatcagtgtaaccaatgcccaggcacgagctatgttgagaagacattctctcattggccctgacataaaaattacttat

SEQ ID NO: 121 - DNA, 288 bp, Homo Sapiens
aatcagcccaggcgggtggaactctggagagaaccaagcaaatccttaggcatcagcattgttggtggacgagggatggggagtcggctaagcaatggagaagtgatgaggggcattttcatcaaacatgttctggaagatagtccagctggcaaaaatggaaccttgaaacctggagatagaatcgtagaggtggatggaatggacctcagagatgcaagccatgaacaagctgtggaagccattcggaaagcaggcaaccctgtagtctttatggtacagagcatt

SEQ ID NO: 122 - DNA, 261 bp, Homo Sapiens
ggcgagctgcatatgattgaactggagaaaggtcatagtggtttgggcctaagtcttgctgggaacaaagaccgatccaggatgagtgtcttcatagtggggattgatccaaatggagctgcaggaaaagatggtcgattgcaaattgcagatgagcttctagagatcaatggtcagattttatatggaagaagtcatcagaatgcctcatcaatcattaaatgtgccccttctaaagtgaaaataatttttatcagaaat

SEQ ID NO: 123 - DNA, 257 bp, Homo Sapiens
aaaaatgtgcaacatctggagcttcccaaggatcaggggggtttgggtattgctatcagcgaagaagatacactcagtggagtcatcataaagagcttaacagagcatggggtagcagccacggatggacgactcaaagtcggagatcagatactggctgtagatgatgaaattgttgttggttaccctattgaaaagtttattagccttctgaagacagcaaagatgacagtaaaacttaccatccatgctgagaa

SEQ ID NO: 124 - DNA, 261 bp, Homo Sapiens
ggctgcgaaacaaccatcgagatttccaaagggcgaacagggctgggcctgagcatcgttgggggttcagacacgctgctgggtgccattattatccatgaagtttatgaagaaggagcagcatgtaaagatggaagactctgggctggagatcagatcttagaggtgaatggaattgacttgagaaaggccacacatgatgaagcaatcaatgtcctgagacagacgccacagagagtgcgcctgacactctacagagat

SEQ ID NO: 125 - DNA, 257 bp, Homo Sapiens
gacaccctcactattgagctgcagaagaagccgggaaaaggcctaggattaagtattgttggtaaaagaaacgatactggagtatttgtgtcagacattgtcaaaggaggaattgcagatgccgatggaagactgatgcagggagaccagatattaatggtgaatggggaagacgttcgtaatgccacccaagaagcggttgccgctttgctaaagtgttccctaggcacagtaaccttggaagttggaagaatcaa

SEQ ID NO: 126 - DNA, 270 bp, Homo Sapiens
cagggattaagaacagtcgaaatgaaaaagggccctactgactcactgggaatcagcatcgctggaggagtaggcagcccacttggtgatgtgcctatatttattgcaatgatgcacccaactggagttgcagcacagacccaaaaactcagagttggggataggattgtcaccatctgtggcacatccactgagggcatgactcacacccaagcagttaacctactgaaaaatgcatctggctccattgaaatgcaggtggttgctgga

SEQ ID NO: 127 - DNA, 265 bp, Homo Sapiens
  variation 117,164,171,189,242: n = a, c, g, or t
cctcaatgtaagtctattacactagagcgaggaccagatggcttaggcttcagtatagttggaggatatggcagccctcatggagacttacccatttatgttaaaacagtgtttgcaaagggagcagcctctgaanacggacgtctgaaaaggggcgatcagatcattgctgtcaatgggcanagtctanaaggagtcacccatgaanaagctgttgccatccttaaacggacaaaaggcactgtcactttgatggttctctctt

SEQ ID NO: 128 - DNA, 297 bp, Homo Sapiens
ggcaaacccttttttacacgaaacccttctgagttgaaaggcaagttcattcacacaaagctgcggaaaagcagtcgtggctttggcttcacggtggttggaggggatgaacctgatgagtttctccagatcaagagcttggtcctagatggtcctgctgcattggatggcaagatggaaacaggggatgtgattgtaagtgtgaatgacacctgtgttttgggacacacacatgctcaagttgtgaagatcttccagtccatccccattggtgccagcgttggacctgaactctgc

SEQ ID NO: 129 - DNA, 246 bp, Homo Sapiens
ccagaactcataactgttcatattgtcaaagggccaatgggctttggttttactatcgcagacagtcctggtgggggtggccaaagagtgaaacagattgttgacagcccaaggtgccgaggcctgaaagaaggggatctcatagtggaagttaataagaagaacgtgcaggccctaactcacaaccaagtggtggatatgctggttgagtgtcctaagggaagtgaggtcacattgttggtgcaa

SEQ ID NO: 130 - DNA, 258 bp, Homo Sapiens
taccaggaacaggacatcttcctctggagaaaagagactggatttggatttaggattctgggtggaaatgaaccaggggaacctatttatattggtcacatcgtaccactgggtgctgctgatactgacggccgcctgaggtctggagatgaattaatctgtgtggatgggacgccagtaattggaaaatcacaccagcttgtggtccagcttatgcaacaagctgccaagcaaggccacgtcaatctcacggtgcgg

SEQ ID NO: 131 - DNA, 297 bp, Homo Sapiens
gtgcagccctacgacgtggagatccggcgcggggagaacgagggcttcggcttcgtcatcgtgtcctcggtgagcaggcccgaagcaggcacgacctttggcaatgcatgtgtggctatgcctcacaaaataggtcggattattgaggggagccctgctgaccgctgtggcaagctgaaagtaggagaccggatcttggcagtaaatggatgttccatcaccaacaaatcccattcagacattgtgaacctaatcaaggaagcgggaaacacagttaccctccgcatcatttcctgg

SEQ ID NO: 132 - DNA, 114 bp, Homo sapiens
gatgaactgcctgctggttgggaaaagattgaagaccctgtctatggtatctactatgtagaccacatcaacaggaagacacaatatgagaacccggttctagaagccaaacgg

SEQ ID NO: 133 - protein, 38 aa, Homo sapiens
DELPAGWEKIEDPVYGIYYVDHINRKTQYENPVLEAKR

SEQ ID NO: 134 - DNA, 260 bp, Homo Sapiens
ggacaggaaatgattatagaaatatccaagggacgttcagggcttggtctcagcattgtgggaggaaaagacacacccttgaatgctatagttatccatgaagtctatgaagaaggggcagcagccagagatggaagactttgggctggtgaccagatattagaggttaatggggttgacctgaggaactccagccacgaagaagccatcacagccctgaggcagaccccccagaaggtgcggctggtggtgtatagaga

SEQ ID NO: 135 - DNA, 254 bp, Homo Sapiens
gagattttccctgtggatctgcagaagaaagctggccggggcctgggcctgagcattgttgggaaacgaaatggaagcggagtgtttatttctgacatcgtgaaaggcggagccgcagacctggatgggagattgattcagggagatcagatcttatctgtgaatggggaggacatgagaaatgcctcacaggagacagtggccaccatcctcaagtgtgcacagggacttgtgcagctagagattggaagact

SEQ ID NO: 136 - DNA, 251 bp, Homo sapiens
ggaacaaagcccctgcaggccccaggtttccgaaaaaaaccactcttcacccgggatgcatcccagttgaagggaacattcctcagcaccaccctaaaaaagagcaacatgggctttggatttaccatcattggtggaaacaagcctgatgagtttctgcaggtgaaaagtgtgattccggatgggcctgcagcacaggatggaaaaatggaaacaggtgatgtcattgtctatattaatgaagtttgtgt

SEQ ID NO: 137 - DNA, 240 bp, Homo sapiens
gctgaacttatgaccttaaccattgtgaaaggtgcccagggcttcggcttcactattgccgacagtcctacaggacagcgggtgaaacaaatacttgacattcagggatgccctggcctgtgtgaaggcgacctcattgttgagatcaaccagcagaatgtacagaacctgagccatacagaagtagtggatatacttaaggactgtcccattggaagtgaaacttctttgattatccat

SEQ ID NO: 138 - DNA, 258 bp, Homo sapiens
tataaggaattggatgttcatcttcggaggatggagtctggatttggcttcagaatcctcgggggagatgagcctggacagcctattttgattggagctgtcattgccatgggctcagccgacagagatggccgccttcacccaggagatgagcttgtgtatgttgatgggattccagtagccggcaaaacccaccgctatgtcatcgacctcatgcaccacgcagcccgcaatgggcaggtcaacctcactgtgaga

SEQ ID NO: 139 - DNA, 279 bp, Homo sapiens
cagaccagtgatgtggtcattcaccgcaaagagaatgagggcttcggctttgtcatcatcagctccctgaacaggcctgagtctggatccactataactgtgccccataaaatcggacgcatcattgatgggagtcctgcagatcgctgtgcaaaactaaaagtgggagaccggatcctagcagtgaatggccagtctatcatcaacatgcctcacgctgacatcgtgaagctcatcaaggatgcaggtcttagtgtcacccttcgcatcattcctcag

SEQ ID NO: 140 - DNA, 114 bp, Homo sapiens
gacccattgcctgataactgggaaatggcctatacagagaagggcgaagtctacttcattgaccataacacaaagacaacatcatggctggatccacgacttgcgaaaaaggct

SEQ ID NO: 141 - DNA, 114 bp, Homo sapiens
aatgagcttccatatggctgggaaaaaatcgatgatcccatttatggcacttattatgttgaccacataaatagaaaaacacagtttgaaaatcctgtcctggaagcaaaaagg

SEQ ID NO: 142 - protein, 38 aa, Homo sapiens
DPLPDNWEMAYTEKGEVYFIDHNTKTTSWLDPRLAKKA

SEQ ID NO: 143 - protein, 38 aa, Homo sapiens
NELPYGWEKIDDPIYGTYYVDHINRKTQFENPVLEAKR

SEQ ID NO: 144 - DNA, 258 bp, Homo sapiens
ggtgtccttgttcgagcatcactgaaaaaaagcacaatgggatttggttttactattattggtggagatagacctgatgagttcctacaagtgaaaaatgtgctgaaagatggtcccgcagctcaggatgggaaaattgcaccaggcgatgttattgtagacatcaatggcaactgtgtcctcggtcacactcatgcagatgttgtccagatgtttcaattggtacctgtcaatcagtatgtaaacctcactttatgt

SEQ ID NO: 145 - DNA, 235 bp, Homo sapiens
cctgaactagtgactatccctttgattaagggccctaaagggtttgggtttgcaattgctgacagccctactggacagaaggtgaaaatgatactggatagtcagtggtgtcaaggccttcagaaaggagatataattaaggaaatataccatcaaaatgtgcagaatttaacacatctccaagtggtacaggtgctaaagcagtttccagtaggtgctgatgtaccattgctta

SEQ ID NO: 146 - DNA, 441 bp, Homo sapiens
accaaaactgccaaaatgaaaacagataaaaaggaaaatgcaggaagtttggaggccataaatgagcctattcctcagcctatgccttttccaccgagcattatcaggtcaggatccccaaaattggatccttctgaggtctacctgaaatctaagactttatatgaagataaaccaccaaacaccaaagatttggatgtttttcttcgaaaacaagagtcagggtttggcttcagggtgctaggaggagatcgacctgaccagtctatatatattggggctattattcccctgggagcagctgagaaagatggtcggctccgcccagctgatgaactaatgtgcattgatggaattcctgttaaagggaaatcacacaaacaagtcttggacctcatgacaactgctgctcgaaatggccatgtgttactaactgtcaga

SEQ ID NO: 147 - DNA, 270 bp, Homo sapiens
gagccctatgatgttgtcttgcaacgaaaagaaaatgaaggatttggctttgtcatcctcacctccaaaaacaaaccacctccaggagttattcctcataaaattggccgagtcatagaaggaagtccggctgaccgctgtggaaaactgaaagttggagatcatatctctgcagtgaatgggcagtccattgttgaactgtctcatgataacattgttcagctgatcaaagatgctggtgtcaccgtcacactaacggtcattgctgaa

SEQ ID NO: 148 - DNA, 78 bp, Homo sapiens
tgggaaatggcctacactgacacagggatgatctacttcattgaccacaataccaagacaaccacctggttggatcct

SEQ ID NO: 149 - DNA, 72 bp, Homo sapiens
tgggagaaaatagaggaccctcagtatgggacatactatgttgatcaccttaaccagaaaacccagtttgaa

SEQ ID NO: 150 - protein, 26 aa, Homo sapiens
WEMAYTDTGMIYFIDHNTKTTTWLDP

SEQ ID NO: 151 - protein, 26 aa, Homo sapiens
WEKIEDPQYGTYYVDHLNQKTQFENP

SEQ ID NO: 152 - protein, 86 aa, Artificial Sequence
  Consensus
  VARIANT 2,5,6,9,12,24,25,26,28,29,: Xaa = any amino acid
  VARIANT (15)...(22): Xaa = any amino acid
  VARIANT 31,32,33,34,35,36,53,54,56,57,58,68,69,70,71,75,76,78,79,82,84,85: Xaa = any amino acid
  VARIANT (38)...(51): Xaa = any amino acid
GXLYXXGDXILXVNXXXXXXXXHXXXVXXLXXXXXXVXXXXXXXXXXXXXXLXXGXXXGLGFSIAGGXXXXIFIXXIXXGGXAXXD

SEQ ID NO: 153 - protein, 4 aa, Artificial Sequence
  C-terminal PDZ Consensus motif
GTSL

SEQ ID NO: 154 - protein, 13 aa, Artificial Sequence
  Inhibitor of PDZ1 domain
CPPSPDRDRESIV

SEQ ID NO: 155 - protein, 13 aa, Artificial Sequence
  Inhibitor of PDZ1 domain
CPPSPDRDRESIV

SEQ ID NO: 156 - protein, 38 aa, Homo sapiens
EPLPKNWEMAYTDTGMIYFIDHNTKTTTWLDPRLCKKA

SEQ ID NO: 157 - protein, 38 aa, Homo Sapiens
GELPYGWEKIEDPQYGTYYVDHLNQKTQFENPVEEAKR

SEQ ID NO: 158 - protein, 38 aa, Homo Sapiens
GPLPPGWEKRYDPNGRVYYVDHNTRTTTWEDPRLEGKR

SEQ ID NO: 159 - protein, 38 aa, Homo Sapiens
LPSGWGWEQRKDPHGRTYYVDHNTRTTTWERPQPLPPG

SEQ ID NO: 160 - protein, 38 aa, Homo Sapiens
QPLPPGWERRVDDRRRVYYVDHNTRTTTWQRPTMESVR

SEQ ID NO: 161 - protein, 38 aa, Homo Sapiens
GPLPPGWEKRVDSTDRVYFVNHNTKTTQWEDPRTQGLQ

SEQ ID NO: 162 - protein, 38 aa, Homo Sapiens
EPLPEGWEIRYTREGVRYFVDHNTRTTTFKDPRNGKSS

SEQ ID NO: 163 - protein, 38 aa, Homo Sapiens
DALPAGWEQRELPNGRVYYVDHNTKTTTWERPLPPGWE

SEQ ID NO: 164 - protein, 38 aa, Homo Sapiens
RPLPPGWEKRTDPRGRFYYVDHNTRTTTWQRPTAEYVR

SEQ ID NO: 165 - protein, 37 aa, Homo Sapiens
GPLPPGWEKRQDNGRVYYVNHNTRTTQWEDPRTQGMI

SEQ ID NO: 166 - protein, 38 aa, Homo Sapiens
PALPPGWEMKYTSEGVRYFVDHNTRTTTFKDPRPGFES

SEQ ID NO: 167 - protein, 38 aa, Homo Sapiens
GPLPENWEMAYTENGEVYFIDHNTKTTSWLDPRCLNKQ

SEQ ID NO: 168 - protein, 38 aa, Homo Sapiens
PGLPSGWEERKDAKGRTYYVNHNNRTTTWTRPIMQLAE

SEQ ID NO: 169 - protein, 38 aa, Homo Sapiens
SFLPPGWEMRIAPNGRPFFIDHNTKTTTWEDPRLKFPV

SEQ ID NO: 170 - protein, 38 aa, Homo Sapiens
GPLPPGWEERIHLDGRTFYIDHNSKITQWEDPRLQNPA

(19) United States
(12) Patent Application Publication
(10) Pub. No.: US 2005/0112552 A1
(43) Pub. Date: May 26, 2005

(54) Identification and isolation of novel polypeptides having PDZ domains and methods of using same

(75) Inventors: Juan Herrero, Milford, NJ (US); Gregorio Pirozzi, Brookeville, MD (US); Albert Uveges, Hatboro, PA (US)

Correspondence Address: MORGAN LEWIS & BOCKIUS LLP, 1111 PENNSYLVANIA AVENUE NW, WASHINGTON, DC, 20004, US

(73) Assignee: AxCell Biosciences Corporation

(21) Appl. No.: 10/785,819

(22) Filed: Feb. 23, 2004

Related U.S. Application Data
(63) Continuation of application No. 09/723,810, filed on Nov. 28, 2000, now abandoned.
(60) Provisional application No. 60/211,215, filed on Jun. 13, 2000.

(51) Int. Cl. C12Q 1/70; C12Q 1/68; G01N 33/53

(52) U.S. Cl. 435/5; 435/7.1; 435/6

(57) ABSTRACT

The invention described herein encompasses the identification and isolation of polypeptides having PDZ domains or functional equivalents thereof. Various methods of use of these polypeptides are described including, but not limited to, targeted drug discovery. The invention also includes nucleotide sequences encoding novel PDZ domains and the proteins encoded thereby. The invention additionally provides for various peptide recognition units that bind to PDZ domains. The present invention also encompasses nucleotide sequences encoding novel WW domains and the polypeptides encoded thereby.

### 1. FIELD OF THE INVENTION

The present invention is directed to the identification and isolation of polypeptides having PDZ domains or functional equivalents thereof. Various methods of use of these polypeptides are described including, but not limited to, targeted drug discovery. Also provided are nucleotide sequences encoding novel PDZ domains and the proteins encoded thereby. Additionally provided are various peptide recognition units that bind to PDZ domains. The present invention also provides for nucleotide sequences encoding novel WW domains and the polypeptides encoded thereby.

### 2. BACKGROUND OF THE INVENTION

### 2.1. Functional Domains in Proteins

Many biological processes involve the specific binding of proteins to one another. Examples of such processes are siginal transduction, transcription, DNA replication, cytoskeletal organization, membrane transport, etc. In many cases it has been shown that specific binding is mediated by small portions of the proteins involved and that these portions can function to a large extent independently of the rest of the proteins. Such independent portions of proteins, mediating specific recognition or binding of one protein by another, have come to be called “functional domains”. Different functional domains have been characterized to a variety of levels of understanding. Some of these are described below.

Src homology 2 domains (SH2) domains are short (about 100 residues) amino acid sequences that were originally found in the non-membrane bound tyrosine kinase Src. Since then they have been shown to occur in over 20 other proteins. SH2 domains recognize certain phosphotyrosine-containing sites on proteins. Proteins containing SH2 domains participate in a variety of signalling pathways. For reviews discussing SH2 domains see Pawson, 1995, Nature 373:573-580; Cohen et al., 1995, Cell 80:237-248; Pawson and Gish, 1992, Cell 71:359-362; and Koch et al., 1991, Science 252:668-674.

Src homology 3 (SH3) domains are another class of short (about 60-70 residues) amino acid sequences that were originally found by comparing the amino acid sequence of the Src protein with the sequences of Crk, Phospholipase C-γ, α-Spectrin, Myosin IB, Cdc25, and Fus1 (Lehto et al., 1988, Nature 334:388; Mayer et al., 1988, Nature 332:272-275; Stahl et al., 1988, Nature 332:269-272; Rodaway et al., 1989, Nature 342:624). In addition to Src, over 30 proteins are known to contain SH3 domains and these proteins perform a wide range of functions. SH3 domains have been shown to specifically bind certain proline-rich amino acid sequences (Chen et al., 1993, J. Am. Chem. Soc. 115:12591-12592; Ren et al., 1993, Science 259:1157-1161; Feng et al., 1994, Science 266:1241-1247; Yu et al., 1994, Cell 76:933-945; Sparks et al., 1994, J. Biol. Chem. 269:23853-23856; Sparks et al., 1996, Proc. Natl. Acad. Sci. USA 93:1540-1544). For reviews discussing SH3 domains see Pawson, 1995, Nature 373:573-580; Cohen et al., 1995, Cell 80:237-248; Pawson and Gish, 1992, Cell 71:359-362; Koch et al., 1991, Science 252:668-674.

The WW domain is a small functional domain found in a large number of proteins from a variety of species including humans, nematodes, and yeast. Its name is derived from the observation that two tryptophan residues, one in the amino terminal portion of the WW domain and one in the carboxyl terminal portion, are almost invariably conserved. At about 30 to 40 amino acids in length, the WW domain is quite small for a functional domain, as most functional domains tend to be from 50 to 150 residues long. Often a WW domain will be flanked by stretches of amino acids rich in histidine or cysteine; these stretches might be metal-binding sites. The center of WW domains is quite hydrophobic; however, sprinkled throughout the rest of the domain are a high number of charged residues. These features are characteristic of functional domains involved in protein-protein interactions (Bork and Sudol, 1994, Trends in Biochem. Sci. 19:531-533).

Based upon their study of various WW domains, André and Springael, 1994, Biochem. Biophys. Res. Comm. 205:1201-1205 (“Andre and Springael”) proposed the following consensus sequence for WW domains:
Trp-Xaa-Xaa-Xaa-Xaa-Xaa-Xaa-Xaa-Gly-(Lys/Arg)-Xaa-(Tyr/Phe)-(Tyr/Phe)-Xaa-(Asn/Asp)-Xaa-Xaa-(Thr/Ser)-(Lys/Arg)-Xaa-(Thr/Ser) -(Thr/Gln/Ser)-Trp-Xaa-Xaa-Pro (SEQ ID NO:1) where Xaa represents any amino acid and bold letters represent highly conserved amino acids. André and Springael's analysis of WW domains led them to conclude that WW domains lack α-helical content, instead possessing a central β-strand region flanked by unstructured regions. Other studies predict a structure of β-strands containing charged residues flanking a hydrophobic core composed of four aromatic residues (Chen and Sudol, 1995, Proc. Natl. Acad. Sci. USA 92:7819-7823, and references cited therein).

The WW domain has been found in a wide variety of proteins of varying function. Despite this diversity of function, it appears that most proteins containing WW domains for which a function is known are involved in either cell signalling and growth regulation or organization of the cytoskeleton. For example, the WW domain-containing protein dystrophin belongs to a family of cytoskeletal proteins that includes α-actin and β-spectrin. Mutations in dystrophin are responsible for Duchenne and Becker muscular dystrophies. The dystrophin gene gives rise to a family of alternatively spliced transcripts, the longest of which encodes a protein having four domains: (1) a globular, actin-binding region; (2) 24 spectrin-like repeats; (3) a cysteine-rich Ca⁺² binding region; and (4) a carboxyl terminal globular region. This transcript encodes a protein having a WW domain that is located between the spectrin-like repeats and the Ca⁺² binding region. The fact that this WW domain is in an area that has been shown to bind β-dystroglycan suggests that WW domains may be involved in protein-protein interactions (Bork and Sudol, 1994, Trends in Biochem. Sci. 19:531-533).

Utrophin, a protein having 70% sequence homology to dystrophin, and, like dystrophin, capable of forming tetrameres via its spectrin-like repeats, also possesses a WW domain. Utrophin and dystrophin are believed to be involved in membrane stability and the transmission of contractile forces to the extracellular environment (Bork and Sudol, 1994, Trends in Biochem. Sci. 19:531-533).

YAP is a protein that was discovered by virtue of its binding to the SH3 domain of the proto-oncogene Yes (Sudol, 1994, Oncogene 9:2145-2152). Murine YAP was found to have two WW domains; interestingly, chicken and human YAP each have only a single WW domain (Sudol et al., 1995, J. Biol. Chem. 270:14733-14741). The screening of a cDNA expression library with bacterially produced glutathione S-transferase fusion proteins of the WW domain from YAP has resulted in the isolation of WBP-1 and WBP-2, two proteins that specifically bind the YAP WW domain (Chen and-Sudol, 1995, Proc. Natl. Acad. Sci. USA 92:7819-7823). Comparison of the amino acid sequences of WBP-1 and WBP-2 revealed a homologous proline-rich region in each protein containing the motif Pro-Pro-Pro-Pro-Tyr (SEQ ID NO:2). As few as ten residues containing this motif have been shown to confer upon a fusion protein the ability to specifically bind the YAP WW domain (Chen and Sudol, 1995, Proc. Natl. Acad. Sci. USA 92:7819-7823). This binding was highly specific; the motif bound to the YAP WW domain but not to the WW domain from dystrophin or to a panel of SH3 domains.

RSP5 is a protein of yeast that is involved in the phenomenon of nitrogen catabolite inactivation whereby a number of permeases that import nitrogenous compounds into the cell are inactivated when yeast are exposed to a nitrogen source such as NH₄⁺. RSP5 probably interacts with the transcription factor SPT3 since certain alleles of RSP5 can complement mutations in SPT3 (Eisenmann et al., 1992, Genes Dev. 6:1319-1331).

RSP5 contains three WW domains in its amino terminus and appears to be a homolog of the vertebrate protein Nedd-4. Nedd-4, a protein which possesses three WW domains and is believed to play a role in embryonic development and the differentiation of the central nervous system in mouse (Kumar et al., 1992, Biochem. Biophys. Res. Comm. 185:115-1161). The 6 total WW domains of RSP5 and Nedd-4 share 30% amino acid sequence identity and 50% similarity. The carboxyl terminal domains of both RSP5 and Nedd-4 are homologous to the carboxyl terminal domain of E6-AP, a human ubiquitin- protein ligase (André and Springael). A region of RSP5 known as HECT can form a high energy thioester bond with ubiquitin, arguing that RSP5 is a ubiquitin-protein ligase (Scheffner et al., 1995, Cell 75:495-505; Huibregste et al., 1995, Proc. Natl. Acad. Sci. USA 92:2563-2567).

Another yeast protein, ess1, contains a WW domain and is thought to be involved in cytokinesis and/or cell separation (Hanes et al., 1989, Yeast 5:55-72).

A search of protein databases, using the WW domains of Nedd-4 and RSP5, identified two proteins of unknown function, YKLO12W from Saccharomyces cerevesiae and Z22176 from Caenorhabditis elegans, each containing two WW domains at their amino terminus (André and Springael).

Among other proteins having WW domains: the rat transcription factor FE65 possesses an amino terminal activation region that includes a WW domain (Bork and Sudol, 1994, Trends in Biochem. Sci. 19:531-533); the human protein KIAA-143 has 4 WW domains and shares other regions of sequence similarity with RSP5, and may be the human version of mouse Nedd-4 (Hoffman and Bucher, 1995, FEBS Lett. 358:153-157); and the human protein HUMORF1, although of unknown function, has a roughly 350 amino acid region which is homologous to GTPase-activating proteins (André and Springael).

PDZ domains, also known as GLGF (SEQ ID NO:3) repeats or DHR (Disks-large homology region) domains, are a class of modular protein binding domains that were originally identified as three repeated regions of homology of about 100 amino acids in the brain specific post-synaptic density protein, PSD-95, which contain the conserved motif Gly-Leu-Gly-Phe (GLGF) (SEQ ID NO:3) (Cho et al, 1992, Neuron 9:929-942; and Kistner et al., 1993, J. Biol. Chem. 268:4580-4583). The term PDZ domain is derived from the names of three proteins containing such domains (PSD-95; the Drosophila Disks-large tumor suppressor protein DlgA (Woods et al., 1991, Cell 66:451-464); and the epithelial tight-junction zona occludens protein ZO-1 (Itoh et al., 1993, J. Cell. Biol. 121:491-502; Cho et al., 1992, Neuron 9:929-942; reviewed by Gramperts, S. A., 1996, Cell 84:659-662). To date, more than 50 proteins have been identified that contain PDZ domains including evolutionary conserved homologs found in bacteria, yeast, and plants (See generally, Pontig, C., 1997, Protein Sci. 6:464-468). PDZ domains are often found in protein structures at the plasma membrane and in proteins involved in signal transduction pathways. Additionally, the majority of proteins containing PDZ domains appear to be associated with the cytoskeleton at the cell cortex and may function as scaffold or assembly proteins to organize components of signal transduction pathways into spatially distinct units (Kim et al., 1996, Neuron 17:103-113; and Kim et al., 1995, Nature 378:85-88). However, at least one PDZ domain protein, LCAF/IL-16, is secreted, suggesting that the function of PDZ domains may not be limited to the cell cortex (Cruikshank et al., 1996, Proc. Natl. Acad. Sci. USA 91:5109-5113). For review, see e.g., Fanning et al., 1996, Curr. Topics Membranes 43:211-235.

PDZ domains have been shown to bind with high specificity to several ion channels and surface receptors containing the C-terminal consensus peptide motif: Xaa-(Ser/Thr)-Xaa-Val-COOH (SEQ ID NO:4), where Xaa can be any amino acid (Sheng M., 1996, Neuron 17:575-578; Kim et al., 1996, Neuron 17: 103-113; Kim et al., 1995, Nature 378:85-88; Neithammer et al., 1996, J. Neurosci. 16:2157-2163; Gramperts et al., 1996, Cell 84:659-662). Analysis of the crystal structure of the third PDZ domain from hdlg (the human homolog of DlgA) and that for the third PDZ domain from PSD-95 in the presence and absence of bound cognate peptide ligand, have revealed that the two PDZ domains share a similar compact globular structure of six β strands and two α-helices. This crystal structure analysis further revealed that an important feature of the third PDZ domain of PSD-95 and other PDZ domains is the amino acid sequence Gly-Leu-Gly-Phe (SEQ ID NO:3) which forms part of the hydrophobic pocket that binds to the C-terminal carboxylate group of peptides and is commonly referred to as the carboxylate-binding loop (Doyle et al., 1996, Cell 85:1067-1076). The crystal structure also revealed that the last four amino acids of the peptide ligand (Gln-Thr-Ser-Val) (SEQ ID NO:6) bind to the third PDZ domain of PSD-95 within this hydrophobic pocket. More specifically, the crystal structure analysis revealed that specific hydrogen bonds are formed between the amino acids of the ligand (Gln, Thr and Val) and the carboxylate-binding loop as well as with other side chains of residues of the PDZ domain. The hydrophobic pocket on the surface of the PDZ domain is filled by the side chain of the terminal valine, accounting for the requirement for this hydrophobic amino acid at the very C-terminus of the peptide. Further side chain interactions explain the specific recognition of serine or threonine at the −2, and glutamine at the −3 positions. The penultimate (−1) residue of the peptide, however, makes only backbone contact with the PDZ domain, and may account for the observation that the −1 residue of the Glu-Ser-Ile-Val (SEQ ID NO:7) sequence of the K⁺ channel C-terminus may be substituted without impairing PSD-95 binding despite the conservation of aspartate at the −1 position in Shaker and NR2 proteins (Kim et al., 1995, Nature 378:85-88).

While the three-dimensional X-ray structure of the third PDZ domains of hdlg and PSD-95 have revealed that at least four residues at the peptide C-terminus are clearly involved in specific PDZ binding, it cannot be presumed that each member of the extensive list of polypeptides in the databases that terminate with the sequence Xaa-(Ser/Thr)-Xaa-Val-COOH (SEQ ID NO:4), where Xaa is any amino acid will bind a PDZ domain (noted by Kornau et al., 1995, Science 269:1737-1740). Moreover, C-terminal hydrophobic domains other than valine may be accommodated by the PDZ domain, since the inward K⁺ channel subunit Kir2.3 (Glu-Ser-Ala-Ile) (SEQ ID NO:5) has been shown to bind PSD-95 (Cohen et al., 1996, Neuron 17:759-767). See Fanning et al., 1996, Curr. Topics Membranes 43:211-235.

Biochemical analyses using both in vivo and in vitro binding assays suggest that PDZ domains are modular protein-binding domains that have at least three distinct mechanisms for binding: as discussed above, PDZ domains may bind to a specific recognition sequence at the carboxyl termini of proteins, alternatively, PDZ domains may bind to internal sequences of the protein which are not PDZ domains, or they may dimerize with other PDZ domains.

Several examples of the interactions between PDZ domains and the carboxyl termini of proteins have been reported. For example, the first two of the three PDZ domains of PSD-95, Chapsyn 110 and hdlg, have been shown to bind to the carboxyl terminus of subunits of N-methyl-D-aspartate (NMDA) receptor and the Shaker-type potassium channel (Brenman et al., 1996, J. Neurosci. 16:7407-7415; Kornau et al., 1995, Science 269:1737-1740; (Kim et al., 1995, Nature 378:85-88; Kim et al., 1996, Neuron 17:103-113; Neithammer et al., 1996, J. Neurosci. 16:2157-2163; Müller et al., 1996, Neuron 17:255-265; Shieh et al., 1996, Neuron 16:991-998). Interestingly, interactions with PSD-95 have been shown to result in clustering of both K⁺ channels and NMDA receptors, (Kim et al., 1995, Nature 378:85-88; Kim et al., 1996, Neuron 17:103-113).

In another example of interaction between PDZ domains and the carboxyl terminal ends of target proteins, the second PDZ domain of hdlg, a membrane-associated guanylate kinase containing three PDZ domains, has been shown to interact with the carboxyl terminus of APC, the product of the adenomatous polyposis coli tumor suppressor gene which is often mutated in colorectal tumors and is believed to be involved in signal transduction (Matsumine et al., 1996, Science 272:1020-1023 reviewed in Gumbiner, B., 1995, Curr. Opin. Cell Biol. 7:634-640). Like APC, the Drosophila homolog of hdlg called DlgA, was originally identified as a tumor suppressor protein (Woods et al., 1991, Cell 66:451-464). These observations suggest that hdlg and APC might function together in a signal transduction pathway leading to the suppression of cell growth.

In a further example of the interaction between PDZ domains and the carboxyl terminal ends of target proteins, the intracellular protein tyrosine phosphatase PTPL1/FAP1, having six PDZ domains, has been shown to bind to the carboxyl-terminal end of Fas. Fas is a transmembrane protein of the tumor necrosis factor receptor family that can mediate apoptotic signals in many cell types through a “death domain” in the intracellular part of the molecule (Sato et al., 1995, Science 268:411-415). Interactions between Fas and PTPL1/FAP1 are mediated through one of the six PDZ domains of PTPL1/FAP1, and a peptide corresponding to only five amino acid residues at the carboxyl-terminal end of the Fas receptor (Ile-Gln-Ser-Leu-Val-COOH) (SEQ ID NO:8) has been demonstrated to be sufficient and necessary for a specific and strong binding to these PDZ domains (Sato et al., 1995, Science 268:411-415). Interestingly, deletion of the carboxyl terminal 15 residues of Fas has been shown to lead to a potentiated apoptotic response, indicating that the carboxyl terminus of Fas is involved in negative regulation of the apoptotic signal (Itoh et al., 1993, J. Biol. Chem. 268:10932-10937).

The second distinct binding mechanism of PDZ domains is exemplified by InaD, a multivalent adaptor protein involved in Drosophila that acts as a scaffold for different proteins involved in visual signal transduction. InaD contains five PDZ domains and has been shown to interact with an internal cytoplasmic motif (Ser-Thr-Val) of the photoreceptor TRP (Shieh et al., 1996, Neuron 16:991-998; Tsunado et al., 1997, Nature 388:243-249). This cytoplasmic motif is positioned nine residues from the carboxyl terminus of TRP (Shieh et al., 1996, Neuron, 16:991-998), suggesting a binding modality in which the consensus binding sequence is located internally within the binding protein.

PDZ domains have also been shown to form heterotypic dimers. For example, the second PDZ 2 domain of PSD-95 has been shown to bind directly to the single PDZ domain of neuronal nitric oxide synthase (nNOS), a protein that appears to modulate synaptic transmission in the central nervous system (Kornau et al., 1995, Science 269:1737-1740; Stucker et al., 1997, Nat. Biotech. 15:336-340; Brenman et al., 1996, Cell 84:757-767; Brenman et al., 1996, J. Neurosci. 16:7407-7415). Activation of NMDA receptors and other calcium permeable channels has been shown to stimulate nNOS (reviewed by Garthwaite et al., 1995, Annv. Rev. Physiol. 57:683-706). The interaction of PSD-95 with both NMDA receptors and nNOS suggests that PSD-95 as a complex, binds and associates these receptors with their signal transduction machinery. nNos has also been shown to associate with α1 syntrophin through PDZ-PDZ interactions (Brenman et al., 1996, Cell 84:757-767). α1 syntrophin is a cytoplasmic component of the dystrophin-glycoprotein complex of muscle-cell cortical proteins.

Novel genes (and thus their encoded protein products) are most commonly identified from cDNA libraries. Generally, an appropriate cDNA library is screened with a probe that is either an oligonucleotide or an antibody. In either case, the probe must be specific enough for the gene that is to be identified to pick that gene out from a vast background of non-relevant genes in the library. It is this need for a specific probe that is the toughest criteria that must be met in methods for identification of novel genes. Another method of identifying genes from cDNA libraries is through use of the polymerase chain reaction (PCR) to amplify a segment of a desired gene from the library. PCR requires that oligonucleotides having sequence homology to the desired gene be available.

If the probe used is a nucleic acid, the cDNA library may be screened without the need for expressing any protein products that might be encoded by the cDNA clones. If the probe used is an antibody, then it is necessary to build the cDNA library into a suitable expression vector. For a comprehensive discussion of the art of identifying genes from cDNA libraries, see Sambrook, Fritsch, and Maniatis, “Construction and Analysis of cDNA Libraries,” Chapter 8 in Cloning, A Laboratory Manual, 2d ed., Cold Spring Harbor Laboratory Press, 1989. See also Sambrook, Fritsch, and Maniatis, “Screening Expression Libraries with Antibodies and Oligonucleotides,” Chapter 12 in Cloning, A Laboratory Manual, 2d ed., Cold Spring Harbor Laboratory Press, 1989.

As an alternative to cDNA libraries, genomic libraries may be probed with a nucleic acid probe. See Sambrook, Fritsch, and Maniatis, “Analysis and Cloning of Eukaryotic Genomic DNA,” Chapter 9 in Cloning, A Laboratory Manual, 2d ed., Cold Spring Harbor Laboratory Press, 1989.

Nucleic acid probes used in screening libraries are often based upon the sequence of a known gene that is thought to be homologous to a gene that one wishes to isolate. The success of the procedure depends upon the degree of homology between the probe and the target gene being sufficiently high. Nucleic acid probes based upon the sequences of known protein domains have had limited value because, while the sequences of the domains are sometimes similar enough to allow for their recognition as shared domains, the homology between them is too low to be able to design nucleic acid probes that can be used to screen cDNA or genomic libraries for genes containing the domains.

PCR may also be used to identify genes from genomic libraries. However, as in the case of using PCR to identify genes from cDNA libraries, this requires that oligonucleotides having sequence homology to the desired gene be available.

COLT (cloning of ligand targets) is a method that is a function based screen that permits the cloning of modular domains based on their ligand-binding activity. Using operationally defined SH3 ligands, human and mouse cDNA expression libraries have been screened to identify new SH3 domain containing proteins (Sparks et al., 1996, Nat. Biotech. 14:741-744; and PCT International Publication WO 96/31625, published Oct. 10, 1996). Similarly, WW domain containing proteins have been cloned using the COLT method and specific WW domain ligands (Pirozzi et al., 1997, J. Biol. Chem. 272:14611-14616; and PCT International Publication WO 97/37223, published Oct. 9, 1997). Both of these domains (SH3 and WW) bind ligands that are proline rich. The inventors of the present invention have no knowledge of COLT techniques being used with other than porline rich recognition units.

Citation of a reference hereinabove shall not be construed as an admission that such is prior art to the present invention.

### 3. SUMMARY OF THE INVENTION

In general, the present invention is directed to the use of the COLT method to identify an exhaustive set of compounds containing PDZ domains through binding to defined PDZ domain ligands.

More specifically, the present invention is directed to a method of identifying a polypeptide or family of polypeptides having a PDZ domain. The basic steps of the method comprise: (a) choosing a recognition unit or set of recognition units having or suspected of having selective affinity for a known PDZ domain(s) of interest; (b) contacting the recognition unit with a plurality of polypeptides; and (c) identifying one or more polypeptides having a selective affinity for the recognition unit, thereby having a functional PDZ domain.

In one particular embodiment of the invention, exhaustive screening for novel proteins having a functional PDZ domain involves an iterative process by which a peptide recognition unit or PDZ ligand is generated by screening an expression library or a combinatorial peptide library for binders to a known PDZ domain and this recognition unit is then used to identify novel PDZ domain containing proteins in a successive expression library screen.

More particularly, the method of the present invention includes choosing a recognition unit having a selective affinity for a known PDZ domain of interest. With this PDZ ligand recognition unit, it has been discovered that a plurality of polypeptides from various sources can be examined such that certain polypeptides having a selective affinity for the recognition unit can be identified. The polypeptides so identified, have been shown to include a PDZ domain; that is, the PDZ domains found are functional or working versions that are capable of displaying the same binding specificity as the PDZ domain of interest. Hence, the polypeptides identified by the present method also possess those attributes of the known PDZ domain of interest which allow these related polypeptides to exhibit the same, similar, or analogous (but functionally equivalent) selective binding affinity characteristics.

In specific embodiments of the present invention, the plurality of polypeptides is obtained from the proteins produced by a cDNA expression library. The binding specificity of the polypeptides which bear a PDZ domain or a functional equivalent thereof for various peptides or recognition units can subsequently be examined, providing a definition of the physiological role of particular PDZ polypeptide/recognition unit interactions.

The present invention also provides polypeptides comprising certain amino acid sequences. Moreover, the present invention also provides nucleic acids, including certain DNA constructs comprising certain coding sequences. Other compositions are likewise contemplated which are products of the methods of the present invention.

The invention also provides recognition units that are specific for PDZ domains. In a particular embodiment, peptides having a PDZ domain binding motif are biotinylated, then complexed with streptavidin or streptavidin-alkaline phosphatase to form multivalent PDZ domain recognition units. These recognition units are used to screen cDNA expression libraries to identify classes of polypeptides containing PDZ domains.

The present invention also provides methods for identifying potential new drug candidates (and potential lead compounds) and determining the specificities thereof. For example, knowing that a polypeptide with a PDZ domain and a recognition unit, e.g., a binding peptide, exhibit a selective affinity for each other, one may attempt to identify a drug that can exert an effect on the polypeptide-recognition unit interaction, e.g., either as an agonist or as an antagonist (inhibitor) of the interaction. With this assay, then, one can screen a collection of candidate “drugs” for the one exhibiting the most desired characteristic, e.g., the most efficacious in disrupting the interaction or in competing with the recognition unit for binding to the polypeptide. Depending on the desired physiological response, one may want to identify a drug that has broad specificity for the functional domain and thereby has potentially broad physiological effects. Alternatively, one may want a highly specific drug that targets a particular functional domain-ligand interaction while not affecting others. The PDZ domain-recognition unit pairs provided by the current invention allow this to be accomplished.

The present invention also provides a method of targeted drug discovery based on the observed effects of a given drug candidate on the interaction between a PDZ recognition unit-PDZ domain containing polypeptide pair or a recognition unit and a “panel” of related polypeptides each with a copy or a functional equivalent of (e.g., capable of displaying the same binding specificity as) a PDZ domain.

In addition, the present invention also provides certain assay kits and methods of using these assay kits for screening drug candidates. In a specific embodiment of the present invention, the assay kit comprises: (a) a polypeptide containing a PDZ domain; and (b) a recognition unit having a selective affinity for the PDZ domain polypeptide. In another specific embodiment of the invention, the assay kit comprises: (a) a plurality of polypeptides, each polypeptide containing a PDZ domain, preferably of a different sequence: and (b) at least one recognition unit having a selective affinity for each of the PDZ domains of the plurality of polypeptides.

### 4. DESCRIPTION OF THE FIGURES

FIG. 1 is a schematic representation of the general COLT method used to identify polypeptides containing a PDZ domain by screening a plurality of polypeptides using a suitable recognition unit. In the illustration, the plurality of polypeptides is obtained from a cDNA expression library and the recognition units are polypeptides determined by a database search to terminate with the sequence Xaa-Ser/Thr-Xaa-Val-COOH (SEQ ID NO:4), and the extended sequence Xaa-Ser/Thr-Xaa-Yaa-COOH (SEQ ID NO:82) where Xaa is any amino acid and Yaa is a small hydrophobic C-terminal amino acid.

FIG. 2 illustrates a strategy for exhaustively screening an expression library for PDZ domain-containing proteins. A peptide recognition unit is generated by screening an expression library or a combinatorial peptide library for binders to a PDZ domain expressed bacterially as a GST fusion protein. In a second screen, this recognition unit is then used to select PDZ domain-containing proteins represented in a cDNA expression library. A cDNA expression library or combinatorial library is once again used to identify recognition units of PDZ domains identified in the second screen; these recognition units identify overlapping sets of proteins from the expression library. With multiple iterations of this process, it should be possible to clone systematically all PDZ domains represented in a given cDNA expression library.

FIGS. 3A and 3B show an alignment of the 22 novel PDZ domains from the proteins PDZP1, PDZP2, PDZP3, PDZP4, and PDZP5 (SEQ ID NOS:9-23 and 106-118), and the four PDZ domains from KIAA-147 (SEQ ID NOS:24-27), with the third PDZ domain from PSD95 (SEQ ID NO:9). This alignment illustrates the minimal primary sequence homology among the various PDZ domains. Residues in PDZ domain 3 of PSD-95 and corresponding residues in other proteins involved in binding the C-terminus of the peptide (-Gln-Thr-Xaa-Val-COOH; (SEQ ID NO:29) are boxed (Doyle et al., 1996, Cell 85:1067-1076). Residues boxed with a star are those forming hydrogen bonds with the carboxylate loop of the peptide. Boxed residues surround valine of the peptide. Residues binding to threonine of the peptide are boxed with a dot. Residues binding to glutamine of the peptide are boxed with a triangle. (Gln: Glutamine, Thr: Threonine, Val: Valine). Amino acids conserved in >75% of the sequences are shown in the consensus (SEQ ID NO:28). Secondary structure β-sheet (β-A through β-F) are shown as arrows and a helixes (α-A and α-B) as rectangles.

FIG. 4A is a schematic representation of a variety of specificities found in a population of PDZ domain containing polypeptides. Recognition unit A is specific for a group of PDZ domain containing polypeptides represented by circle A. Recognition unit B, on the other hand, has a broader specificity for PDZ domains represented by circles 1, 2, and 3. Subsets of PDZ domains of the B group show affinity also for recognition units B1, B2, and B3 and A. Recognition units B1, B2 and B3 are then used to screen for another group of PDZ containing proteins represented by circles 4, 5, and 6. PDZ domains represented by circle 4 also show affinity for ligands B4 and B5. B4 and B5 are then used to screen, further identifying PDZ domain proteins represented by circles 7, 8, etc.

FIG. 4B illustrates an iterative method whereby new recognition units are chosen based on polypeptides uncovered with the first recognition unit(s). These new recognition units lead to the identification of other related polypeptides, etc., expanding the scope of the study to increasingly diverse members of the related population.

FIGS. 5A and 5B. PDZ domain-peptide interactions shown as cross affinity maps. Recognition unit-biotinylated peptides were tested for their relative binding to individual PDZ domains expressed as GST fusion proteins (See Section 6). The twelve carboxy terminal amino acids of a nonexhaustive list of 46 peptide sequences (SEQ ID NOS:30-75, and 119) that may bind PDZ domains are shown. The first column provides the peptide ligand sequence identifier. The fourth column provides the Genbank accession number. In FIG. 5A, each peptide recognition unit complex was tested for its ability to bind to novel PDZ domains of PDZP2 (domains 2.1, 2.2, 2.3, and 2.4), PDZP3 (domains 3.1 and 3.2), and KIAA-147 (domains 147.1, 147.2, 147.3, and 147.4) expressed as GST fusion proteins. In FIG. 5B, each peptide recognition unit complex was tested for its ability to bind to three PDZ domains of PSD-95 and three domains of Chapsyn expressed as fusion proteins. A minus indicates no binding; a plus indicates binding, with the number of pluses indicating the strength of binding. “nd” indicates not determined. Relative binding was assessed from three independent determinations. All peptide sequences displayed no detectable binding to GST control protein or to bovine serum albumin. For further details, see Section 6.2.

FIG. 6A is a schematic of the proteins encoded by the isolated clones and their modular architecture that make up novel PDZ domain-containing genes PDZP1, PDZP2, PDZP3, PDZP4 and PDZP5. The relative location and size of various modular protein domains including the PDZ, WW, polyglutamine (Poly Q), polyglutamate (Poly E) and guanylate kinase-like (GUK) domains are shown within the protein coding regions of PDZP1, PDZP2, PDZP3, PDZP4, PDZP5, and KIAA-147 (Genbank accession number:D63481). Arrows denote incomplete N and C-terminal coding sequences. aa=amino acids.

FIG. 6B shows schematically the protein encoded by the isolated gene sequence for PDZP1, PDZP2, PDZP3, PDZP4, PDZP5, and the PDZ domains in KIAA-147. The relative location and size of various modular protein domains including the PDZ, WW, polyglutamine (Poly Q), polyglutamate (Poly E) and guanylate kinase-like (GUK) domains are shown within the protein coding regions of PDZP1, PDZP2, PDZP3, PDZP4, PDZP5, and KIAA-147 (Genbank accession number:D63481). Arrows denote incomplete N and C-terminal coding sequences. aa=amino acids.

FIGS. 7A and 7B depict the nucleotide sequence of PDZP1, a novel human gene (SEQ ID NO:75). Nucleotide sequences encoding PDZ domains are highlighted in bold and underlined. Nucleotide sequences encoding PDZ domains are as follows: PDZP1.1, nucleotides 150 to 404 (SEQ ID NO:120); PDZP1.2, nucleotides 579 to 866 (SEQ ID NO:121); PDZP1.3, nucleotides 1077 to 1337 (SEQ ID NO:122); PDZP1.4, nucleotides 1476 to 1732 (SEQ ID NO:123); PDZP1.5, nucleotides 1914 to 2174 (SEQ ID NO:124); PDZP1.6, nucleotides 2205 to 2461 (SEQ ID NO:125); PDZP1.7, nucleotides 2613 to 2678 (SEQ ID NO:126); and PDZP1.8, nucleotides 3006 to 3270 (SEQ ID NO:127).

FIG. 8 depicts the amino acid sequence of PDZP1, a novel human protein (SEQ ID NO:76). Amino acid sequences of the PDZ domains are highlighted in bold and underlined. Amino acid sequences corresponding to the PDZ domains are as follows: PDZP1.1, amino acid residues 50-134 (SEQ ID NO:111); PDZP1.2, amino acid residues 193-288 (SEQ ID NO:112); PDZP1.3, amino acid residues 359-445 (SEQ ID NO:10); PDZP1.4, amino acid residues 492-576 (SEQ ID NO:11); PDZP1.5, amino acid residues 638-724 (SEQ ID NO:113); PDZP1.6, amino acid residues 735-819 (SEQ ID NO:114); PDZP1.7, amino acid residues 871-960 (SEQ ID NO:115); and PDZP1.8, amino acid residues 996-1083 (SEQ ID NO:116).

FIG. 9 depicts the nucleotide sequence of PDZP2 (SEQ ID NO:77). Nucleotide sequences encoding PDZ domains are highlighted in bold and underlined. Nucleotide sequences encoding PDZ domains are as follows: PDZP2.1, nucleotides 359 to 655 (SEQ ID NO:128); PDZP2.2, nucleotides 911 to 1156 (SEQ ID NO:129); PDZP2.3, nucleotides 1421 to 1678 (SEQ ID NO:130); and PDZP2.4, nucleotides 1892-2188 (SEQ ID NO:131). Nucleotide sequences encoding the WW domain is highlighted in bold. Nucleotides 67-120 (SEQ ID NO:132) encode the PDZP2.WW2 domain.

FIG. 10 depicts the amino acid sequence of PDZP2 (SEQ ID NO:78). Amino acid sequences of the PDZ domains are highlighted in bold and underlined. Amino acid sequences corresponding to the PDZ domains are as follows: PDZP2.1, amino acid residues 134-219 (SEQ ID NO:12); PDZP2.2, amino acid residues 305-386 (SEQ ID NO:13); PDZP2.3, amino acid residues 475-559 (SEQ ID NO:14); and PDZP2.4, amino acid residues 632-730 (SEQ ID NO:15). The amino acid sequence of the WW domain is highlighted in bold. The WW domain, PDZP2.WW2, is composed of amino acid residues 23-60 (SEQ ID NO:133).

FIG. 11 depicts the nucleotide sequence of PDZP3, a novel human gene (SEQ ID NO:79). Nucleotide sequences encoding PDZ domains are highlighted in bold and underlined. Nucleotide sequences encoding PDZ domains are as follows: PDZP3.1, nucleotides 118 to 377 (SEQ ID NO:134) and PDZP3.2, nucleotides 409 to 663 (SEQ ID NO:135).

FIG. 12 depicts the amino acid sequence of PDZP3, a novel human protein (SEQ ID NO:80). The amino acid sequences of the PDZ domains are highlighted in bold and underlined. Amino acid sequences corresponding to the PDZ domains are as follows: PDZP3.1, amino acid residues 103-189 (SEQ ID NO:16) and PDZP3.2, amino acid residues 200-284 (SEQ ID NO:17).

FIG. 13 depicts the nucleotide sequence of PDZP4, a novel human gene (SEQ ID NO:98). Nucleotide sequences encoding PDZ domains are highlighted in bold and underlined. Nucleotide sequences encoding PDZ domains are as follows: PDZP4.1, nucleotides 548-798 (SEQ ID NO:136); PDZP4.2, nucleotides 1157-1396 (SEQ ID NO:137); PDZP4.3, nucleotides 1634-1891 (SEQ ID NO:138); and PDZP4.4, nucleotides 2063-2341 (SEQ ID NO:139). Nucleotide sequences encoding WW domains are highlighted in bold. Nucleotide sequences encoding WW domains are as follows: PDZP4.WW1, nucleotides 259-372 (SEQ ID NO:140); and PDZP4.WW2, nucleotides 397-510 (SEQ ID NO:141).

FIG. 14 depicts the amino acid sequence of PDZP4, a novel human protein (SEQ ID NO:99). Amino acid sequences of the PDZ domains are highlighted in bold and underlined. Amino acid sequences corresponding to the PDZ domains are as follows: PDZP4.1, amino acid residues 207-292 (SEQ ID NO:18); PDZP4.2, amino acid residues 386-465 (SEQ ID NO:19); PDZP4.3, amino acid residues 545-630 (SEQ ID NO:20); and PDZP4.4, amino acid residues 688-780 (SEQ ID NO:21). Amino acid sequences corresponding to the WW domain are highlighted in bold. The amino acid sequences corresponding to the WW domains are as follows: PDZP4.WW1, amino acid residues 87-124 (SEQ ID NO:142); and PDZP4.WW2, amino acid residues 133-170 (SEQ ID NO:143).

FIG. 15 depicts the nucleotide sequence of PDZP5, a novel human gene (SEQ ID NO:100). Nucleotide sequences encoding PDZ domains are highlighted in bold and underlined. Nucleotide sequences encoding PDZ domains are as follows: PDZP5.1, nucleotides 742-999 (SEQ ID NO:144); PDZP5.2, nucleotides 1246-1480 (SEQ ID NO:145); PDZP5.3, nucleotides 1507-1947 (SEQ ID NO:146); and PDZP5.4, nucleotides 2068-2337 (SEQ ID NO:147). Nucleotide sequences encoding WW domains are highlighted in bold. Nucleotide sequences encoding WW domains are as follows: PDZP5.WW1, nucleotides 421-498 (SEQ ID NO:148); and PDZP5.WW2, nucleotides 559-630 (SEQ ID NO:149).

FIG. 16 depicts the amino acid sequence of PDZP5, a novel human protein (SEQ ID NO:101). Amino acid sequences of the PDZ domains are highlighted in bold and underlined. Amino acid sequences corresponding to the PDZ domains are as follows: PDZP5.1, amino acid residues 248-333 (SEQ ID NO:22); PDZP5.2, amino acid residues 416-495 (SEQ ID NO:23); PDZP5.3, amino acid residues 564-649 (SEQ ID NO:117); and PDZP5.4, amino acid residues 690-779 (SEQ ID NO:118). Amino acid sequences corresponding to the WW domains are highlighted in bold. The amino acid sequences corresponding to the WW domains are as follows: PDZP5.WW1, amino acid residues 141-166 (SEQ ID NO:150); and PDZP5.WW2, amino acid residues 187-212 (SEQ ID NO:151).

FIGS. 17A and 17B depict the nucleotide sequence of KIAA-147 (SEQ ID NO:102). Nucleotide sequences encoding PDZ domains are highlighted in bold and underlined. Nucleotide sequences encoding PDZ domains are as follows: KIAA-147.1, nucleotides 1138-1397 (SEQ ID NO:152); KIAA-147.2, nucleotides 2329-2604 (SEQ ID NO:153); KIAA-147.3, nucleotides 2755-2925 (SEQ ID NO:154); and KIAA-147.4, nucleotides 2931-3270 (SEQ ID NO:155).

FIG. 18 depicts the amino acid sequence of KIAA-147 (SEQ ID NO:103). Amino acid sequences of the PDZ domains are highlighted in bold and underlined. Amino acid sequences corresponding to the PDZ domains are as follows: KIAA-147.1, amino acid residues 644-733 (SEQ ID NO:24); KIAA-147.2, amino acid residues 777-868 (SEQ ID NO:25); KIAA-147.3, amino acid residues 919-1011 (SEQ ID NO:26); KIAA-147.4, amino acid residues 1015-1110 (SEQ ID NO:27).

FIG. 19 presents a competitive inhibition analysis of the interaction between the first PDZ domain in PSD-95 and the carboxy terminal peptide sequence of Na channel protein. GST fusion protein containing the first PZD domain of PSD-95 was contacted with a recognition unit-peptide complex corresponding to the 12 carboxyl terminal amino acids of Na⁺ channel protein (FIG. 5A, SEQ ID NO:51) and various concentrations of 1 of 3 different inhibitor peptide complexes. Binding of the dimer inhibitor peptide in the presence of streptavidin alkaline phosphatase/Biotin-Ser-Gly-Ser-Gly-Pro-Pro-Ser-Pro-Asp-Arg-Asp-Arg-Glu-Ser-Ile-Val-COOH (SEQ ID NO:157) is presented as a solid box. Binding of the recognition unit in the presence of a peptide corresponding to the 5 carboxy terminal residues of the Na⁺ channel protein (SEQ ID NO:105) (hatched box), and in the presence of a scrambled version of this 5mer (SEQ ID NO:158) (open box), are also presented. Binding of the biotinylated peptide was quantitated at A₄₀₅ nm. For further details, see Section 6.2.

FIG. 20 shows the sequence alignment of WW domains isolated by COLT methods (SEQ ID NOS:159-170, 133, 150, 151, 142 and 143, respectively). Novel WW domains are shown as PDZP2.WW2, PDZP5.WW1, PDZP5.WW2, PDZP4.WW1, PDZP4.WW2 (SEQ ID NOS:133, 150, 151, 142, and 143, respectively).

FIG. 21 presents a schematic of the coupling of N-methyl-D aspartate receptor (NMDAR) activity to nitric oxide (NO) biosyntnesis.

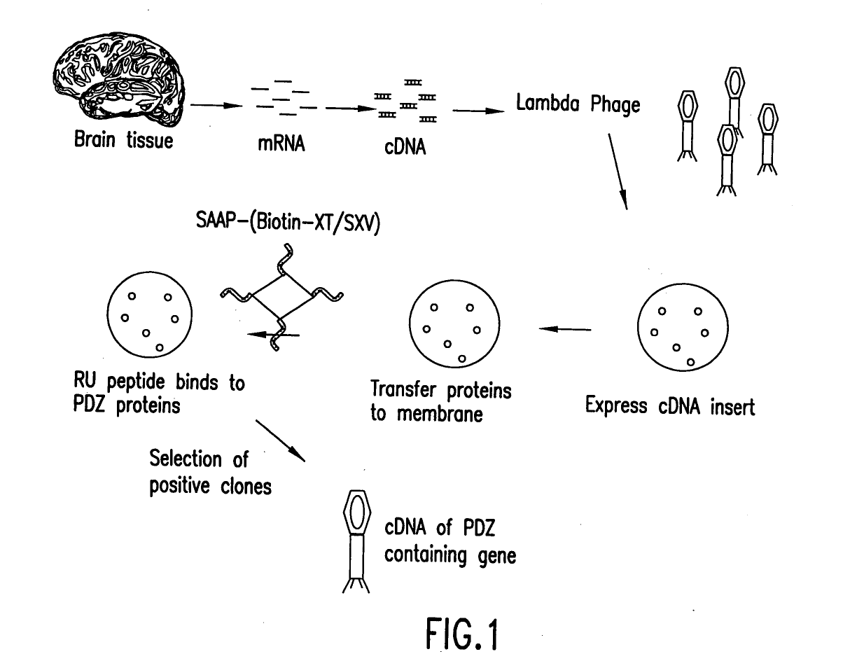

### 5. DETAILED DESCRIPTION OF THE INVENTION

The present invention relates to polypeptides having a PDZ domain and in some instances, a WW domain, methods of identifying and using these polypeptides, PDZ domains and derivatives thereof, and nucleic acids encoding the foregoing. The present invention additionally relates to PDZ domain binding peptides and recognition unit complexes containing these peptides. The detailed description that follows is provided to elucidate the invention further and to assist further those of ordinary skill who may be interested in practicing particular aspects of the invention.

The term “polypeptide” refers to a molecule comprised of amino acid residues joined by peptide (i.e., amide) bonds and includes proteins and peptides. Hence, the polypeptides of the present invention may have single or multiple chains of covalently linked amino acids and may further contain intrachain or interchain linkages comprised of disulfide bonds. Some polypeptides may also form a subunit of a multiunit macromolecular complex. Naturally, the polypeptides can be expected to possess conformational preferences and to exhibit a three-dimensional structure. Both the conformational preferences and the three-dimensional structure will usually be defined by the polypeptide's primary (i.e., amino acid) sequence and/or the presence (or absence) of disulfide bonds or other covalent or non-covalent intrachain interactions.

The polypeptides of the present invention can be any size. The polypeptides can exhibit a wide variety of molecular weights, some exceeding 150 to 200 kilodaltons (kD). Typically, the polypeptides may have a molecular weight ranging from about 5,000 to about 100,000 daltons. Still others may fall in a narrower range, for example, about 10,000 to about 75,000 daltons, or about 20,000 to about 50,000 daltons.

PDZ domains tend to be modular in that such domains may occur one or more times in a given polypeptide or may be found in a family of different polypeptides. When found more than once in a given polypeptide or in different polypeptides, the modular PDZ domain may possess substantially the same structure, in terms of primary sequence and/or three-dimensional conformation, or may contain slight or great variations or modifications among the different versions of the PDZ domain.

What is important, however, is that these related PDZ domains retain at least one of the functional aspects of the known PDZ domain where the investigation began. It is stressed that, indeed, it is this functional similarity among two or more possible versions of a PDZ domain which is identified, defined, and exploited by the present invention. In a preferred aspect, the function of interest is the ability to specifically bind to a molecule (e.g., a peptide recognition unit) of interest.

The present invention provides a general strategy by which recognition units that bind to a PDZ domain-containing protein can be used to screen expression libraries of genes (e.g., cDNA and genomic libraries) systematically to identify novel PDZ domain-containing proteins. In specific embodiments, the recognition units are identified by database searches for sequences having homology to a peptide ligand having binding specificity to PDZ domains. Alternatively, the recognition units are identified by screening a cDNA expression library or random peptide library with a known or novel PDZ domain containing protein to identify ligands that can be used as recognition units. Alternatively, the recognition units are known or potential PDZ domain peptide ligands that can be used as recognition units.

Using the COLT methods, DNA encoding proteins having a PDZ domain are identified by functional binding specificity to PDZ recognition units. By virtue of an ease in specificity of binding requirements and high sensitivity conferred by the COLT methods, many novel, functionally homologous, PDZ domain-containing proteins can be identified. Although not intending to be bound by any mechanistic explanation, this ease in binding specificity and increased sensitivity is believed to be the result of the use of a multivalent recognition unit used to screen the gene library, preferably of a valency greater than bivalent, and more preferably tetravalent or greater. The gene library most preferably screened with a streptavidin-biotinylated peptide recognition unit complex.

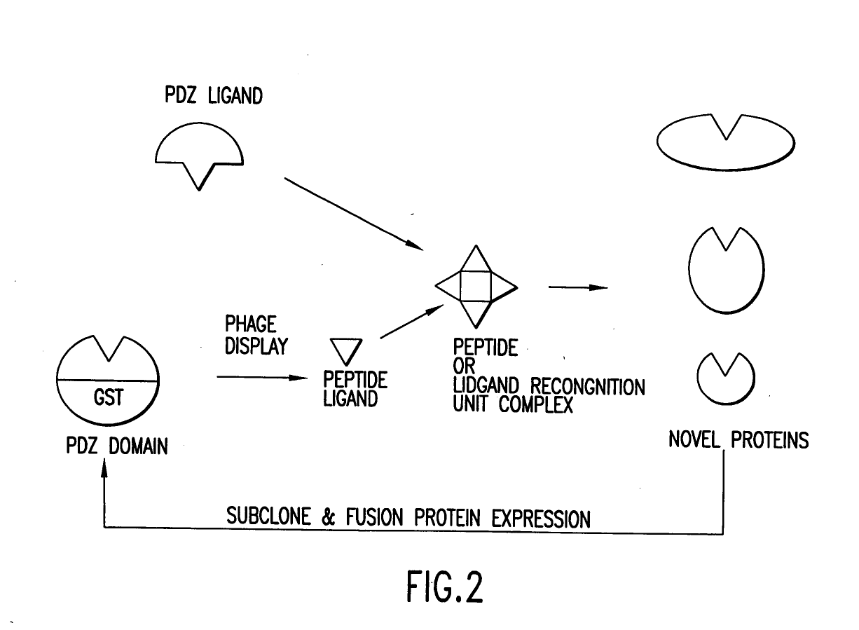

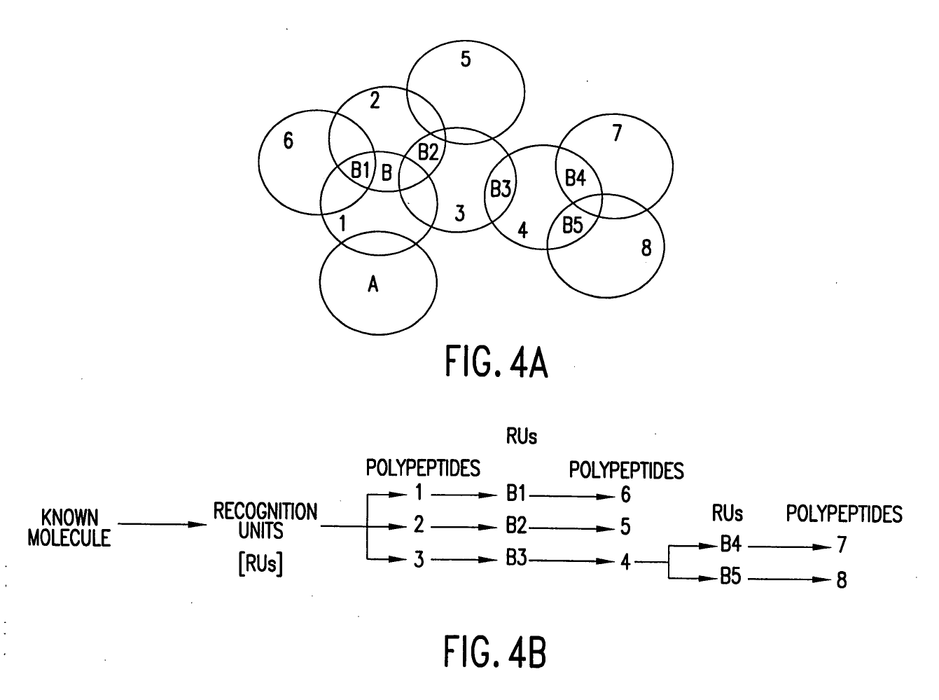

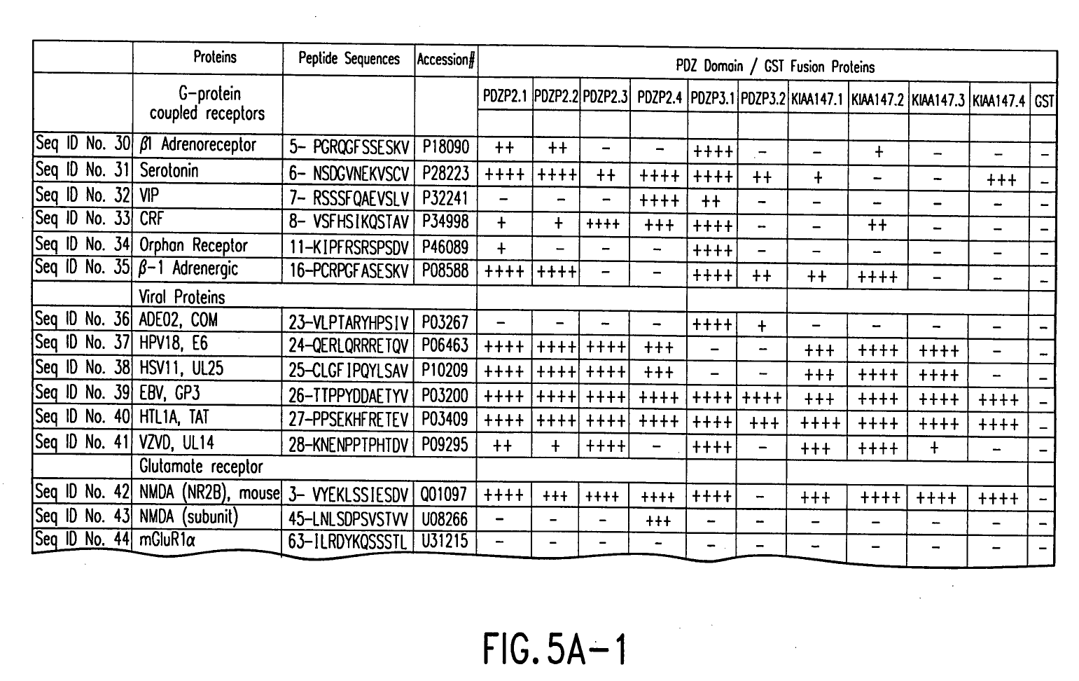

In one particular embodiment of the invention, exhaustive screening for proteins having a PDZ domain involves an iterative process by which recognition units for PDZ domains identified in the first round of screening are used to detect PDZ domain-containing proteins in successive expression library screens (see FIGS. 2, 4A and 4B). This strategy enables one to search “sequence space” in what might be thought of as ever-widening circles with each successive cycle. This iterative strategy can be initiated even when only one PDZ domain-containing protein and recognition unit are available.

The present invention provides novel polypeptides comprising novel PDZ domains and the amino acid sequence and nucleotide sequence encoding these polypeptides and domains. In particular, as presented in Table 1, the present invention provides novel polypeptides PDZP1, PDZP2, PDZP3, PDZP4 and PDZP5 (SEQ ID NOS:76, 78, 80, 99, and 101, respectively) and novel PDZ domains having the amino acid sequence of SEQ ID NOS:10-27 and 111-118. Also provided are nucleic acids encoding these novel polypeptides and PDZ domains (SEQ ID NOS:75, 77, 79, 98, 100, 102, 120-131, 134-139, 144-147 and 152-155). The novel polypeptides and PDZ domains of the present invention can be used to identify and isolate PDZ recognition units that can further be used to identify and isolate additional PDZ domain containing polypeptides by following the procedures set forth infra.

TABLE 1
PDZ DOMAIN CONTAINING POLYPEPTIDES

| Polypeptide | | | | | | | |
|---|---|---|---|---|---|---|---|
| | Amino | | PDZ DOMAIN | | | | |
| | Acid | Nucleotide | | Amino Acid | | Nucleotide | |
| | SEQ ID | SEQ ID | Domain | Position | SEQ ID | Position | SEQ ID |
| PDZP1 | 76 | 75 | 1.1 | 50-134 aa | 111 | 150-404 n | 120 |
| | | | 1.2 | 193-288 aa | 112 | 579-866 n | 121 |
| | | | 1.3 | 359-445 aa | 10 | 1077-1337 n | 122 |
| | | | 1.4 | 492-576 aa | 11 | 1476-1732 n | 123 |
| | | | 1.5 | 638-724 aa | 113 | 1914-2174 n | 124 |
| | | | 1.6 | 735-819 aa | 114 | 2205-2461 n | 125 |
| | | | 1.7 | 871-960 aa | 115 | 2613-2678 n | 126 |
| | | | 1.8 | 996-1083 aa | 116 | 3006-3270 n | 127 |
| PDZP2 | 78 | 77 | 2.1 | 134-219 aa | 12 | 359-655 n | 128 |
| | | | 2.2 | 305-386 aa | 13 | 911-1156 n | 129 |
| | | | 2.3 | 475-559 aa | 14 | 1421-1678 n | 130 |
| | | | 2.4 | 632-730 aa | 15 | 1892-2188 n | 131 |
| PDZP3 | 80 | 79 | 3.1 | 103-189 aa | 16 | 118-377 n | 134 |
| | | | 3.2 | 200-284 aa | 17 | 409-663 n | 135 |
| PDZP4 | 99 | 98 | 4.1 | 207-292 aa | 18 | 548-798 n | 136 |
| | | | 4.2 | 386-465 aa | 19 | 1157-1396 n | 137 |
| | | | 4.3 | 545-630 aa | 20 | 1634-1891 n | 138 |
| | | | 4.4 | 688-780 aa | 21 | 2063-2341 n | 139 |
| PDZP5 | 101 | 100 | 5.1 | 248-333 aa | 22 | 742-999 n | 144 |
| | | | 5.2 | 416-495 aa | 23 | 1246-1480 n | 145 |
| | | | 5.3 | 564-649 aa | 117 | 1507-1947 n | 146 |
| | | | 5.4 | 690-779 aa | 118 | 2068-2337 n | 147 |
| KIAA- | 103 | 102 | 147.1 | 644-733 aa | 24 | 1138-1397 n | 152 |
| 147 | | | | | | | |
| | | | 147.2 | 777-868 aa | 25 | 2329-2604 n | 153 |
| | | | 147.3 | 919-1011 aa | 26 | 2755-2925 n | 154 |
| | | | 147.4 | 1015-1110 aa | 27 | 2931-3270 n | 155 |

The novel PDZ domains may also be used in screening compounds for activity either as agonists or antagonists to the specific PDZ domain-recognition unit interaction.

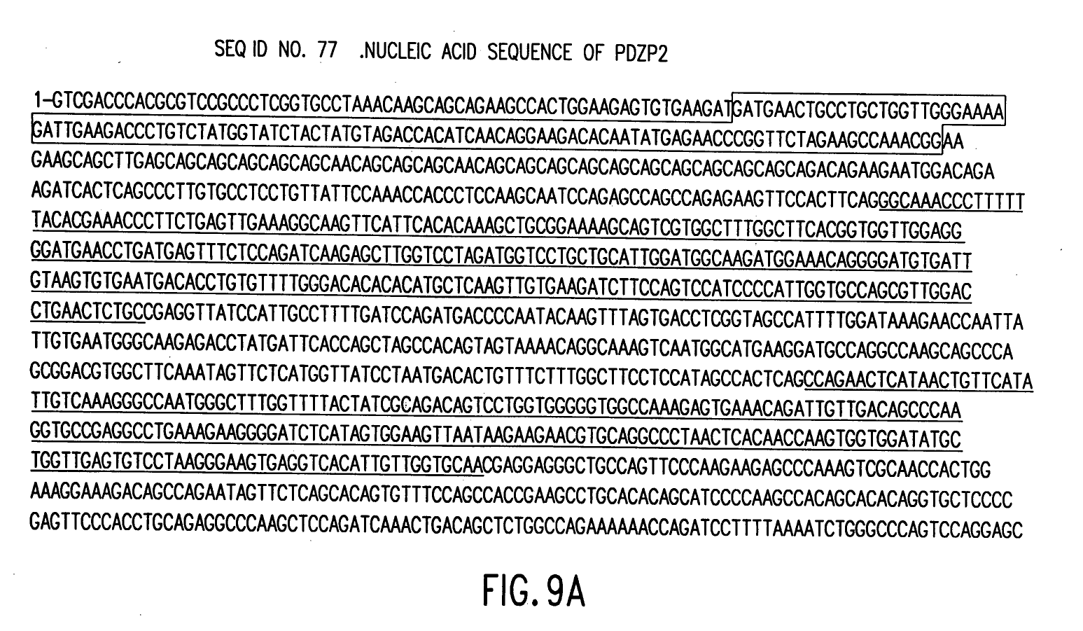

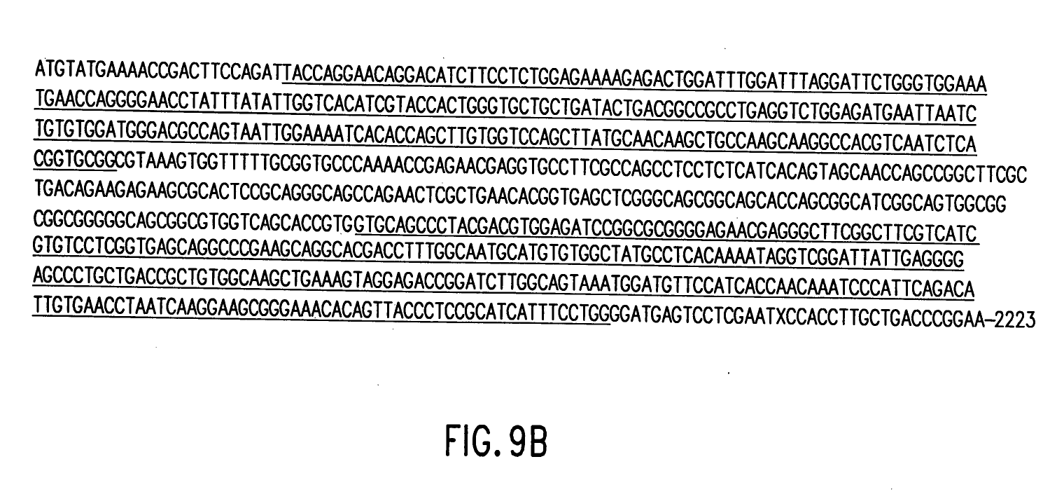

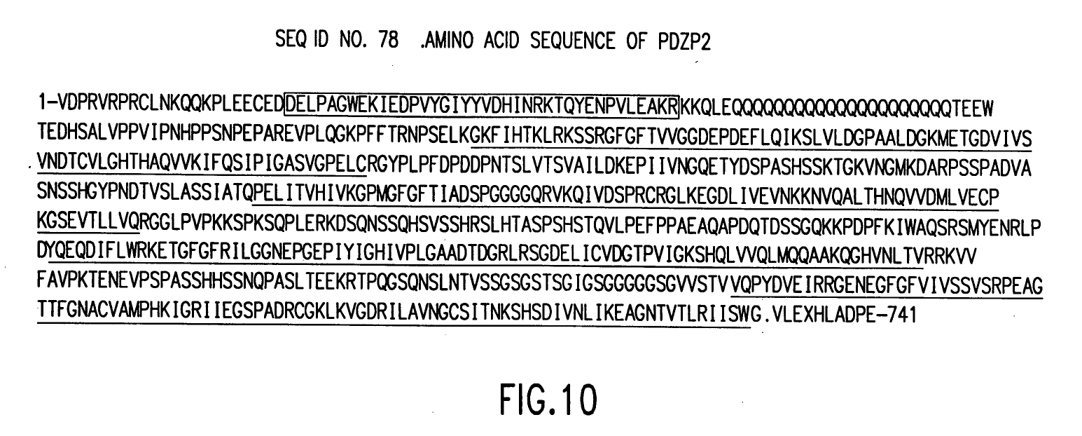

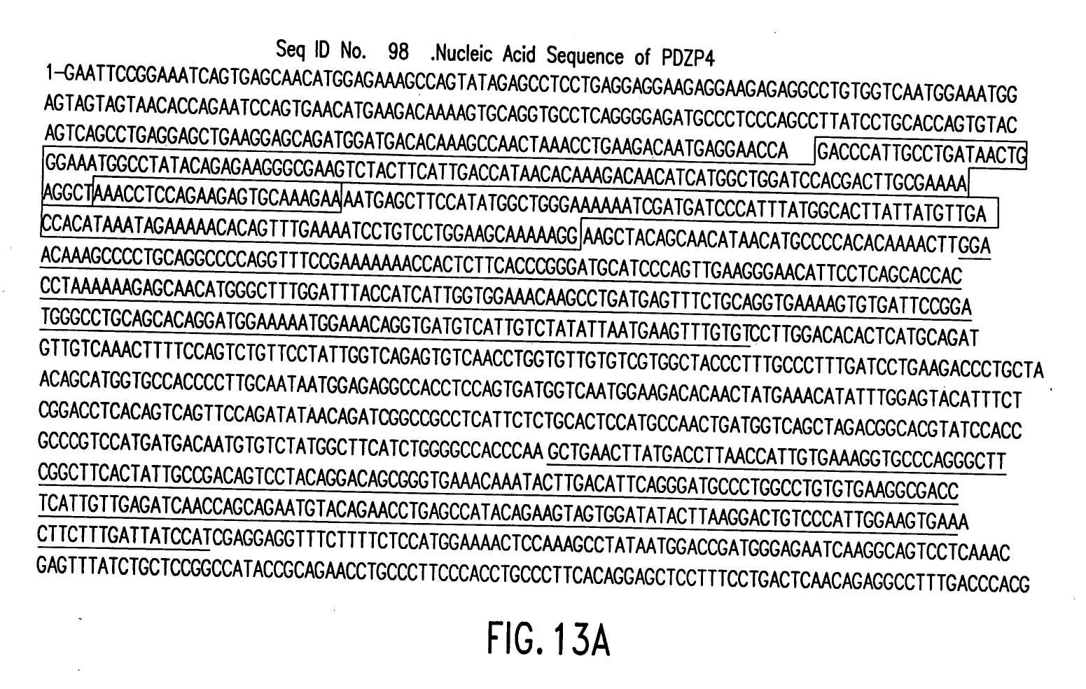

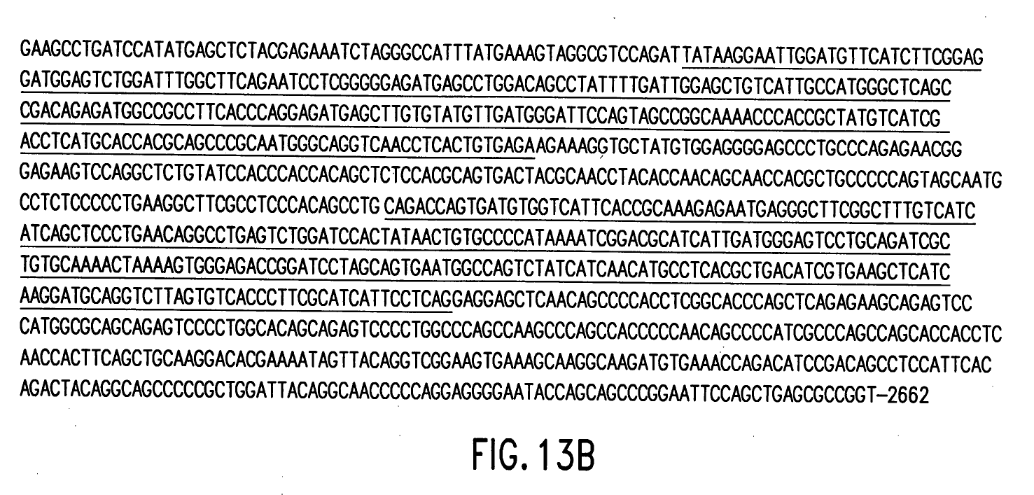

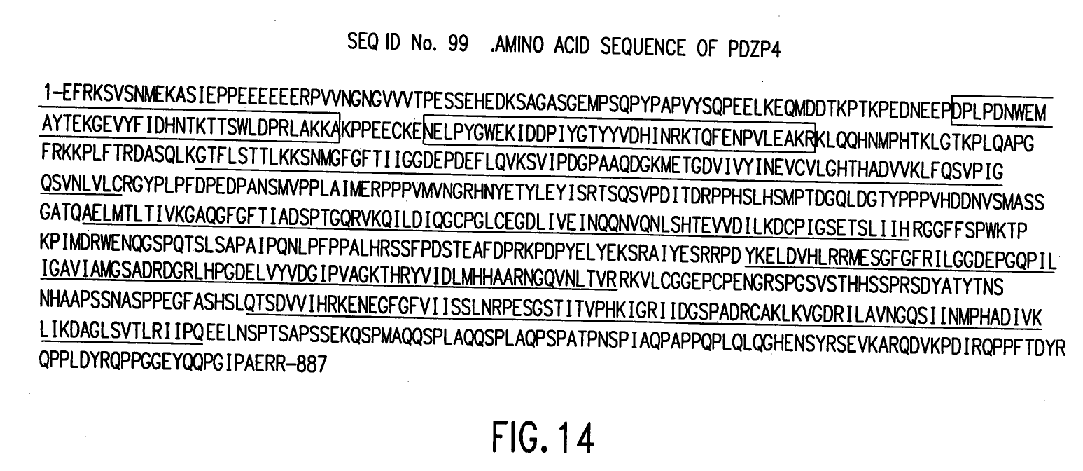

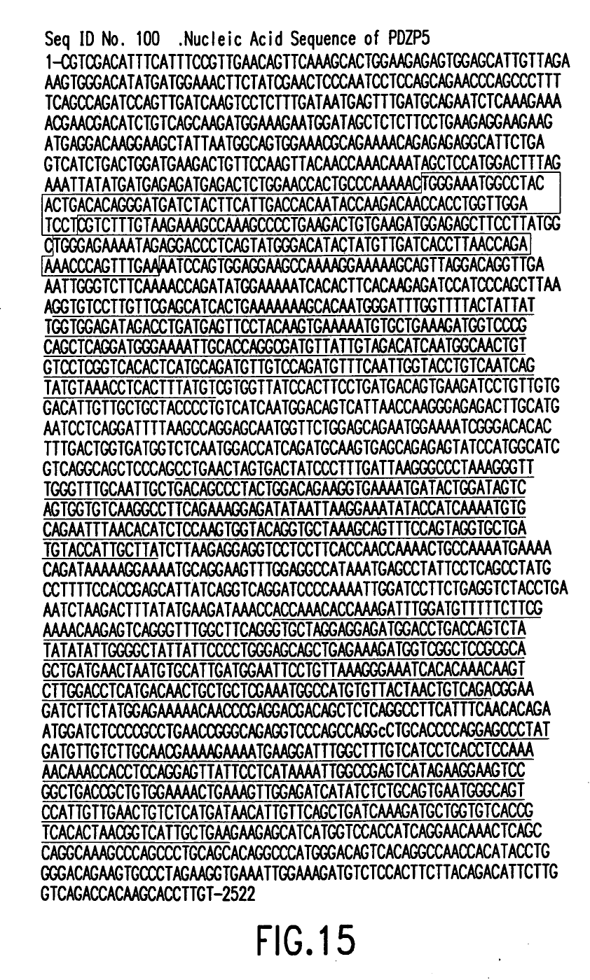

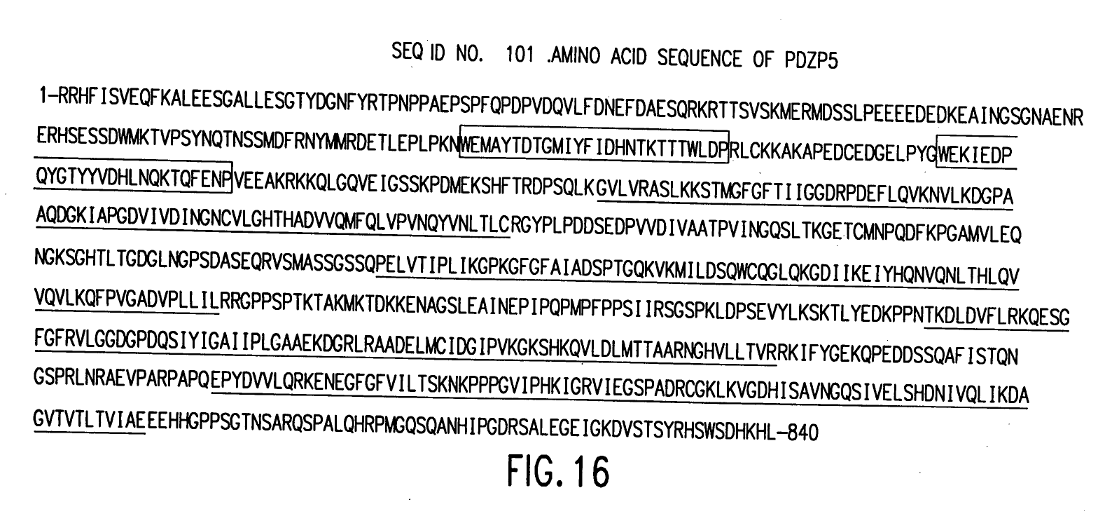

The present invention also provides polypeptides comprising novel WW domains and the amino acid sequence of these WW domains, and the nucleotide sequences encoding these polypeptides. In particular, as presented in Table 2, the present invention provides novel WW domains including PDZP2.WW2, PDZP4.WW1, PDZP4.WW2, PDZP5.WW1, and PDZP5.WW2, having the sequence of: amino acid residues 23-60 (SEQ ID NO:133) of the PDZP2 amino acid sequence as depicted in FIG. 10 (SEQ ID NO:78); amino acid residues 87-124 (SEQ ID NO:142), and amino acid residues 133-170 (SEQ ID NO:143) of the PDZP4 amino acid sequence as depicted in FIG. 14 (SEQ ID NO:99); and amino acid residues 141-161 (SEQ ID NO:150) and amino acid residues 187-212 (SEQ ID NO:151) of the PDZP5 amino acid sequence as depicted in FIG. 16 (SEQ ID NO:101), respectively. Also provided are nucleic acids encoding these novel WW domains. More particularly, the present invention provides for nucleic acids having one or more of the nucleotide sequence of nucleotides 67-120 (SEQ ID NO:132) of the PDZP2 nucleotide sequence as depicted in FIG. 9 (SEQ ID NO:77); nucleotides 259-372 (SEQ ID NO:140) and nucleotides 397-510 (SEQ ID NO:141) of the PDZP4 nucleotide sequence as depicted in FIG. 13 (SEQ ID NO:98); and nucleotides 421-498 (SEQ ID NO:148) and 559-630 (SEQ ID NO:149) of the PDZP5 nucleotide sequence as depicted in FIG. 15 (SEQ ID NO:100). The novel WW domain of the present invention can be used to identify and isolate WW recognition units that can be used to identify and isolate additional WW domain containing polypeptides by following the procedures set forth infra, and substituting the WW domain for the PDZ domain containing protein or nucleic acid encoding the PDZ domain, as generally set forth in PCT International Publication WO 97/37223, published Oct. 9, 1997, which is herein incorporated by reference in its entirety.

The novel WW domains may also be used in screening compounds for activity either as agonists or antagonists to the specific WW domain-recognition unit interaction.

TABLE 2
PDZ DOMAIN POLYPEPTIDES CONTAINING WW DOMAINS

| Polypeptide | | | | | | | |
|---|---|---|---|---|---|---|---|
| | | Nu- | | | | | |
| | | cle- | PDZ DOMAIN | | | | |
| | Amino | otide | | Amino Acid | | Nucleotide | |
| | Acid | SEQ | Do- | | SEQ | | SEQ |
| | SEQ ID | ID | main | Position | ID | Position | ID |
| PDZP2 | 78 | 77 | WW2 | 23-60 aa | 133 | 67-120 n | 132 |
| PDZP4 | 99 | 98 | WW1 | 87-124 aa | 142 | 259-372 n | 140 |
| | | | WW2 | 133-170 aa | 143 | 397-510 n | 141 |
| PDZP5 | 101 | 100 | WW1 | 141-166 aa | 150 | 421-498 n | 148 |
| | | | WW2 | 187-212 aa | 151 | 559-630 n | 149 |

The present invention also provides recognition units which bind to PDZ or WW domains. The recognition units aid in determining PDZ or WW domain specificity. Recognition units also are used for assaying for compounds that will compete with the recognition units for binding to PDZ or WW domains.

The present invention provides assays using novel reagents to classify the binding specificity preferences of various PDZ or WW domains and in turn, the specificity of various PDZ or WW ligands, respectively. The assay and initial data is then used as a drug discovery tool. The ability of prospective drugs to alter the PDZ domain-PDZ ligand or WW domain-WW ligand binding generally or specifically is determined by comparing binding results without the compound present, to those obtained with the compound added to the assay.

### 5.1. Methods for and Discovery of Novel Genes and Polypeptides Containing PDZ Domains

The present invention makes possible the identification of one or more polypeptides (in particular, a “family” of polypeptides, including the target molecule) that contain a PDZ domain that either corresponds to or is the functional equivalent of a known PDZ domain.

The present invention provides a mechanism for the rapid identification of genes (e.g., cDNAs) encoding virtually any PDZ domain. By screening cDNA libraries or other sources of polypeptides for recognition unit binding rather than sequence similarity, the present invention circumvents the limitations of conventional DNA-based screening methods and allows for the identification of highly disparate protein sequences possessing equivalent functional activities. The ability to isolate entire repertoires of proteins containing particular modular PDZ domains is invaluable both in molecular biological investigations of the genome and in bringing new targets into drug discovery programs.

It should likewise be apparent that a wide range of polypeptides having a PDZ domain can be identified by the process of the invention, which process comprises:
(a) contacting a multivalent recognition unit complex comprising potential PDZ domain ligands with a plurality of polypeptides; and
(b) identifying a polypeptide comprising a PDZ domain and having a selective binding affinity for said recognition unit complex.

In a specific embodiment, the process comprises:
(a) contacting a multivalent recognition unit complex with a plurality of polypeptides from which it is desired to identify a polypeptide having a PDZ domain and selective binding affinity for the recognition unit, in which the valency of the recognition unit in the complex is at least two, or at least four, in which the recognition unit comprises potential PDZ domain ligands; and
(b) identifying, and preferably recovering, a polypeptide having a PDZ domain and a selective binding affinity for the recognition unit complex.

In another specific embodiment, the process comprises a method of identifying a polypeptide having a PDZ domain comprising:
(a) contacting a multivalent recognition unit complex, which complex comprises (i) avidin or streptavidin, and (ii) biotinylated recognition units comprising potential PDZ domain ligands, with a plurality of polypeptides from a cDNA expression library, in which the recognition unit is a peptide having in the range of 4 to 150 amino acid residues; and
(b) identifying a polypeptide having a PDZ domain and a selective binding affinity for said recognition unit complex.

In another embodiment, the present invention includes a method of identifying one or more novel polypeptides having a PDZ domain, said method comprising:
(a) searching a database for peptide sequences having homology to a known PDZ domain ligand;
(b) producing a peptide(s) comprising the peptide sequence identified from step (a);
(c) using the peptide(s) of step (b) on a recognition unit complex to screen a source of polypeptides to identify one or more polypeptides containing a PDZ domain;
(d) determining the amino acid sequence of the polypeptides identified in step (c); and
(e) producing the one or more novel polypeptides containing a PDZ domain. In specific embodiments, the database search in step (a) is performed for peptides containing a PDZ domain (e.g., the PDZ consensus sequence (SEQ ID NO:28)) or a PDZ ligand which terminate with the sequence Xaa-(Ser/Thr)-Xaa-Val-COOH (SEQ ID NO:4) or Xaa-(Ser/Thr)-Xaa-Yaa-COOH (SEQ ID NO:82), where Xaa can be any amino acid and Yaa is a small hydrophobic amino acid.

In another embodiment, said polypeptide is a polypeptide containing a PDZ domain produced by a reiterative method comprising:
(a) screening a peptide library and/or an expression library with a PDZ domain to obtain a peptide that binds the PDZ domain;
(b) producing a peptide comprising the binding peptide of (a);
(c) using the peptide of (b) to screen a source of polypeptides to identify one or more polypeptides containing a PDZ domain;
(d) determining the amino acid sequence of the polypeptides identified in step (c); and
(e) producing the one or more polypeptides containing a PDZ domain.

In another embodiment, said polypeptide is a polypeptide containing a PDZ domain produced by a method comprising:
(a) screening a peptide library and/or an expression library with a known PDZ domain to obtain a plurality of peptides that bind the PDZ domain;
(b) determining a consensus sequence for the peptides obtained in step (a);
(c) producing a peptide comprising the consensus sequence;
(d) using the peptide comprising the consensus sequence to screen a source of polypeptides to identify one or more polypeptides containing a PDZ domain;
(e) determining the amino acid sequence of the polypeptides identified in step (d); and
(f) producing the one or more polypeptides containing a PDZ domain.

In another embodiment, the present invention includes a method of identifying one or more novel polypeptides having a PDZ domain, said method comprising:
(a) identifying a peptide having a selective binding affinity for a PDZ domain by screening a cDNA expression library or a peptide library with the PDZ domain;
(b) producing a recognition unit comprising said peptide;
(c) contacting said recognition unit with a source of polypeptides; and
(d) identifying one or more novel polypeptides having a selective binding affinity for said recognition unit, which polypeptides comprise a PDZ domain.

In another specific embodiment, the process comprises a method of identifying a polypeptide having a PDZ domain of interest or a functional equivalent thereof comprising:
(a) screening a random peptide library to identify a peptide that selectively binds a known PDZ domain; and
(b) screening a cDNA or genomic expression library with a recognition unit comprising said peptide or a binding portion thereof to identify a polypeptide that selectively binds said peptide.

In a specific embodiment of the above method, the screening step (b) is carried out by use of said peptide in the form of multiple antigen peptides (MAP) or by use of said peptide cross-linked to bovine serum albumin or keyhole limpet hemocyanin.

In another specific embodiment, the process comprises a method of identifying a polypeptide having a PDZ domain or a functional equivalent thereof comprising:
(a) screening a random peptide library to identify a plurality of peptides that selectively bind a known PDZ domain;
(b) determining at least part of the amino acid sequences of said peptides;
(c) determining a consensus sequence based upon the determined amino acid sequences of said peptides; and
(d) screening a cDNA or genomic expression library with recognition units comprising peptides of the consensus sequence to identify a polypeptide that selectively binds said consensus sequence.

In another specific embodiment, the process comprises a method of identifying a polypeptide having a PDZ domain or a functional equivalent thereof, comprising:
(a) screening a random peptide library to identify a first peptide that selectively binds a known PDZ domain;
(b) determining at least part of the amino acid sequence of said first peptide;
(c) searching a database containing the amino acid sequences of a plurality of expressed natural proteins to identify a protein containing an amino acid sequence homologous to the amino acid sequence of said first peptide; and
(d) screening a cDNA or genomic expression library with a recognition unit comprising the sequence of said protein that is homologous to the amino acid sequence of said first peptide.

The polypeptide identified by the above-described methods thus should contain a PDZ domain of interest or a functional equivalent thereof (that is, have a PDZ domain that is identical, or have a PDZ domain that differs in sequence, but is capable of binding to the same recognition unit). In a particular embodiment, the polypeptide identified is a novel polypeptide. In preferred embodiments, the recognition unit that is used to form the multivalent recognition unit complex is identified from a database search (e.g., for proteins having the motif Xaa-Ser/Thr-Xaa-Val-COOH (SEQ ID NO:4), or more generally Xaa-Ser/Thr-Xaa-Yaa-COOH (SEQ ID NO:82), where Xaa can be any amino acid and Yaa is a small hydrophobic C-terminal amino acid), or isolated or identified from a cDNA expression library or a random peptide library. In a specific embodiment the recognition unit has an amino acid sequence selected from the group consisting of SEQ ID NOS:30-74 and 119.

The present invention provides amino acid sequences encoded by DNA sequences encoding novel proteins containing PDZ domains. The PDZ domains vary in sequence but retain binding specificity to a PDZ domain recognition unit. Also provided are fragments and derivatives of the novel proteins containing PDZ domains as well as DNA sequences encoding the same. It will be apparent to one of ordinary skill in the art that also provided are proteins that vary slightly in sequence from the novel proteins by virtue of conservative amino acid substitutions. It will also be apparent to one of ordinary skill in the art that the novel proteins may be expressed recombinantly by standard methods. The novel proteins may also be expressed as fusion proteins with a variety of other proteins, e.g., glutathione S-transferase.

The present invention provides a purified polypeptide comprising a PDZ domain, said PDZ domain having an amino acid sequence selected from the group consisting of: SEQ ID NOS:10-27, and 111-116. Also provided is a purified DNA encoding the polypeptide (SEQ ID NOS:120-131, 134-139, and 144-147).

Also provided is a purified polypeptide comprising at least one PDZ domain, said polypeptide having an amino acid sequence selected from the group consisting of SEQ ID NOS:76, 78, 80, 99 and 101. Also provided is a purified DNA encoding the polypeptide (SEQ ID NOS:75, 77, 79, 98, 100, and 102).

Also provided is a purified DNA encoding a PDZ domain, said DNA having a sequence selected from the group consisting of SEQ ID NOS:75, 77, 79, 98, and 100. Also provided is a nucleic acid vector comprising this purified DNA. Also provided is a recombinant cell containing this nucleic acid vector.

Also provided is a purified DNA encoding a polypeptide having an amino acid sequence selected from the group consisting of: SEQ ID NOS:76, 78, 80, 99, 101, 103. Also provided is a nucleic acid vector comprising this purified DNA. Also provided is a recombinant cell containing this nucleic acid vector.

Also provided is a purified DNA encoding a polypeptide comprising an amino acid sequence selected from the group consisting of: SEQ ID NOS:10-27 and 111-116.

Also provided is a purified DNA encoding a polypeptide which has at lease one PDZ domain comprising a nucleotide sequence selected from the group consisting of SEQ ID NOS:75, 77, 79, 98, 100, 102.

Also provided is a nucleic acid vector comprising this purified DNA. Also provided is a recombinant cell containing this nucleic acid vector.

Also provided is a purified molecule comprising a PDZ domain of a polypeptide having an amino acid sequence selected from the group consisting of: SEQ ID NOS:10-27, 76, 78, 80, 99, 101, 103 and 111-116.

Also provided is a fusion protein comprising (a) an amino acid sequence comprising a PDZ domain of a polypeptide having the amino acid sequence of SEQ ID NOS:10-27, 111-116, 76, 78, 80, 99, 101, and 103, joined via a peptide bond to (b) an amino acid sequence of at least six, or ten, or twenty amino acids from a different polypeptide. Also provided is a purified DNA encoding the fusion protein. Also provided is a nucleic acid vector comprising the purified DNA encoding the fusion protein. Also provided is a recombinant cell containing this nucleic acid vector. Also provided is a method of producing this fusion protein comprising culturing a recombinant cell containing a nucleic acid vector encoding said fusion protein such that said fusion protein is expressed, and recovering the expressed fusion protein.

The present invention also provides a purified nucleic acid hybridizable to a nucleic acid having a sequence selected from the group consisting of: SEQ ID NOS:75, 77 79, 98, 100 and 102.

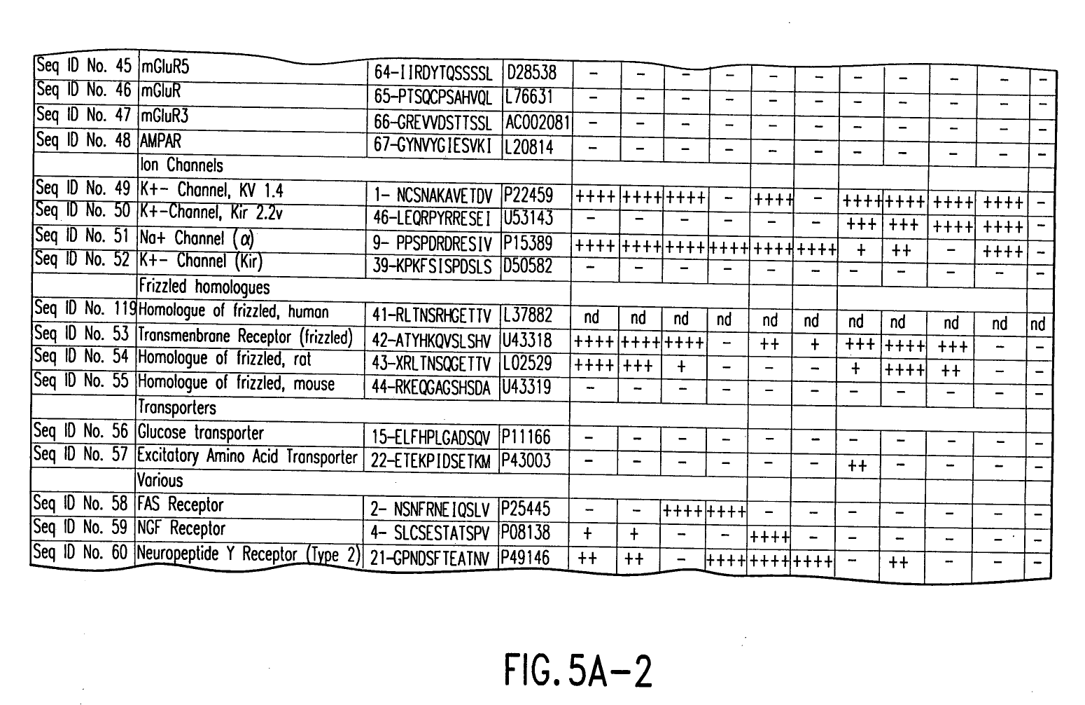

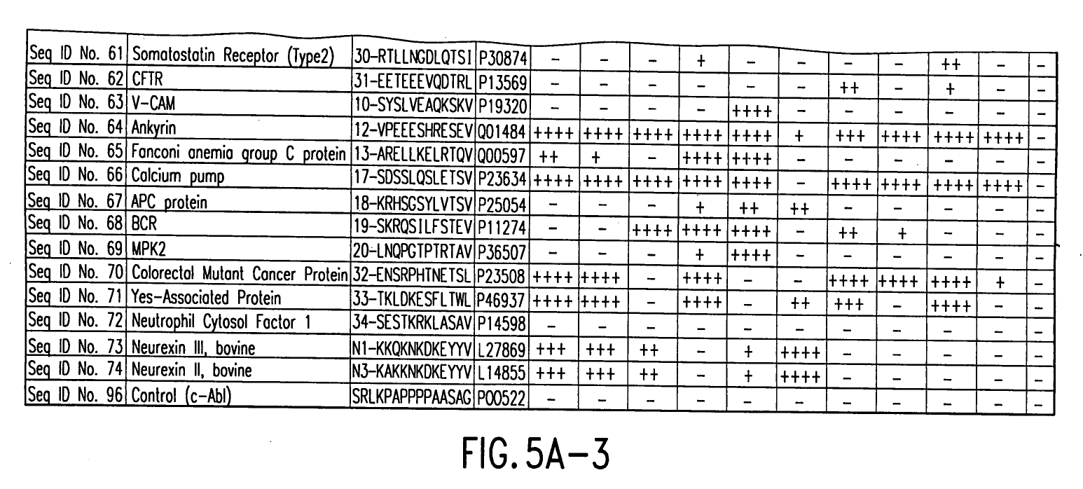

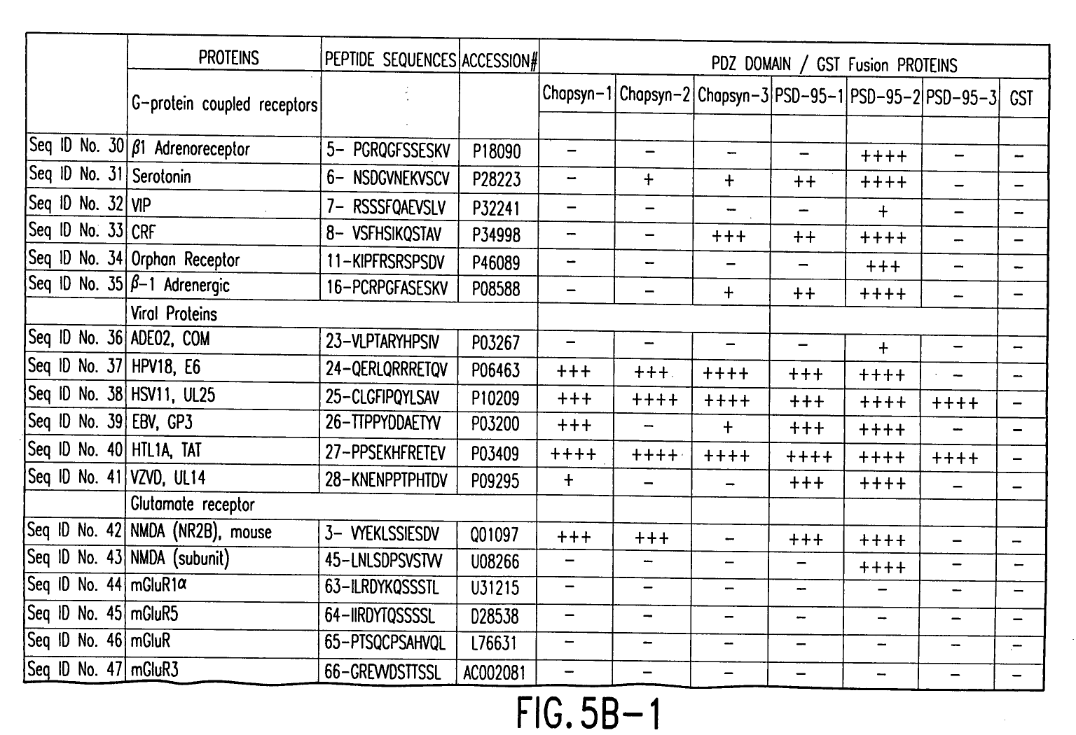

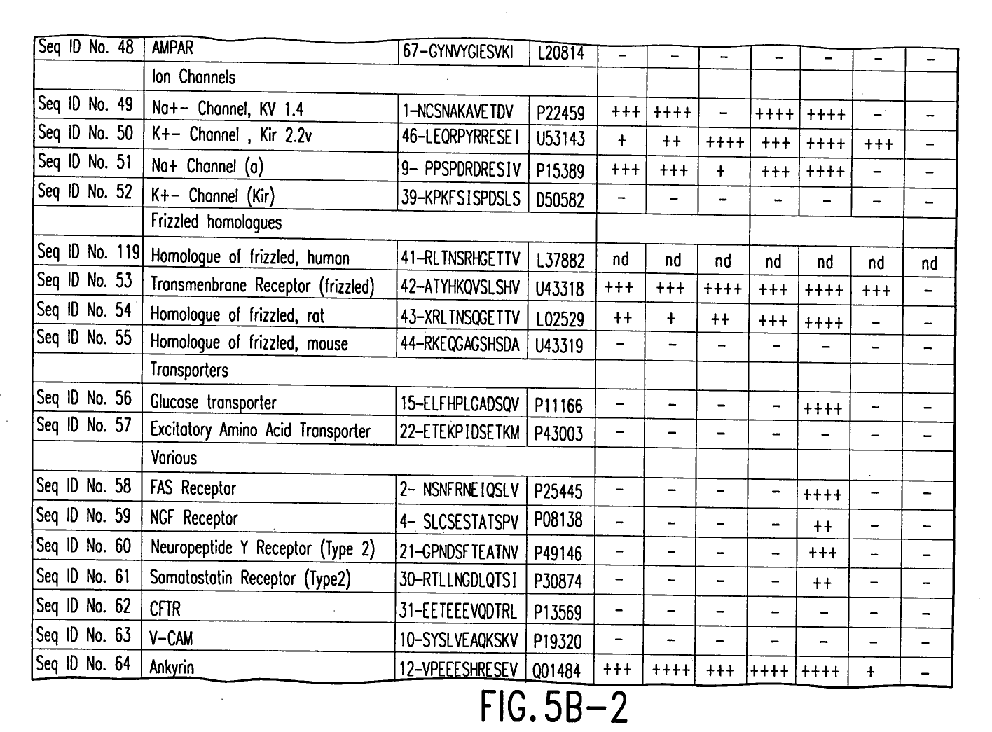

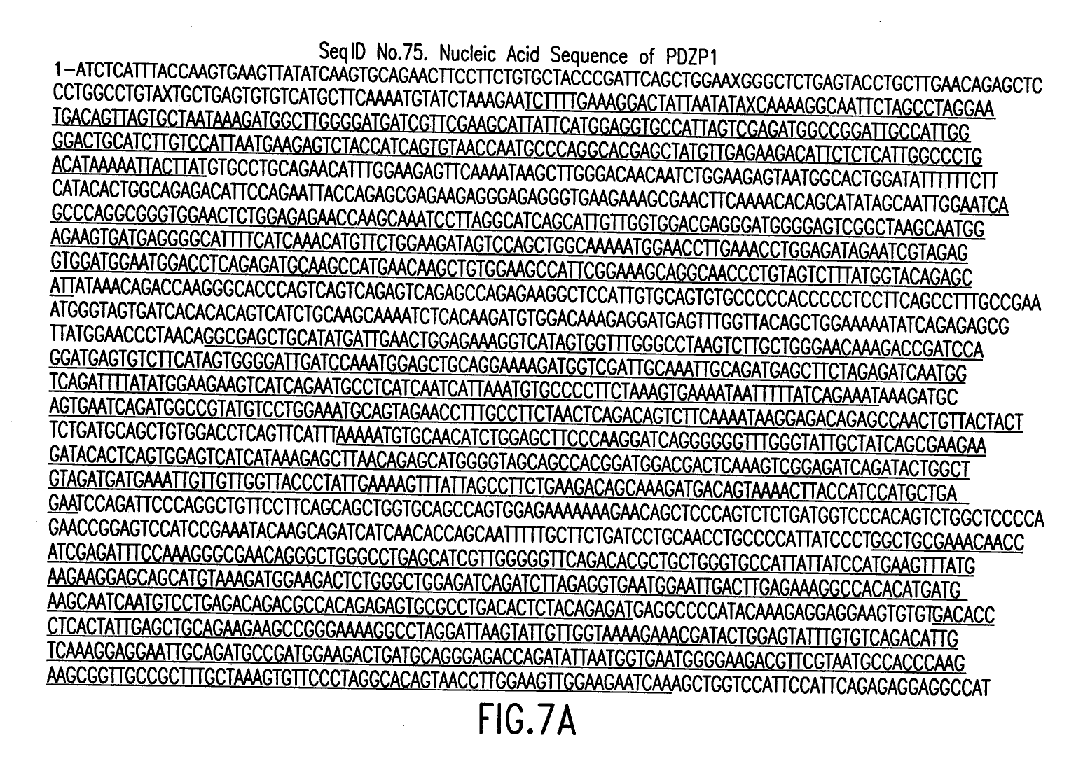

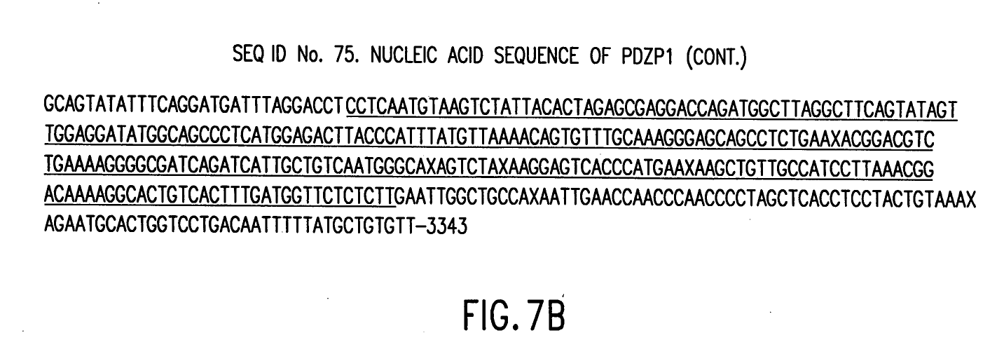

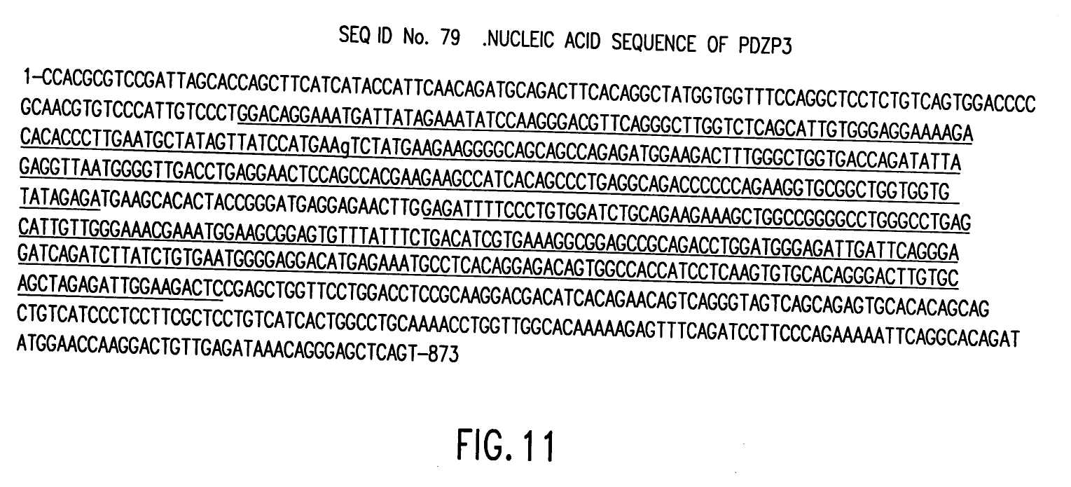

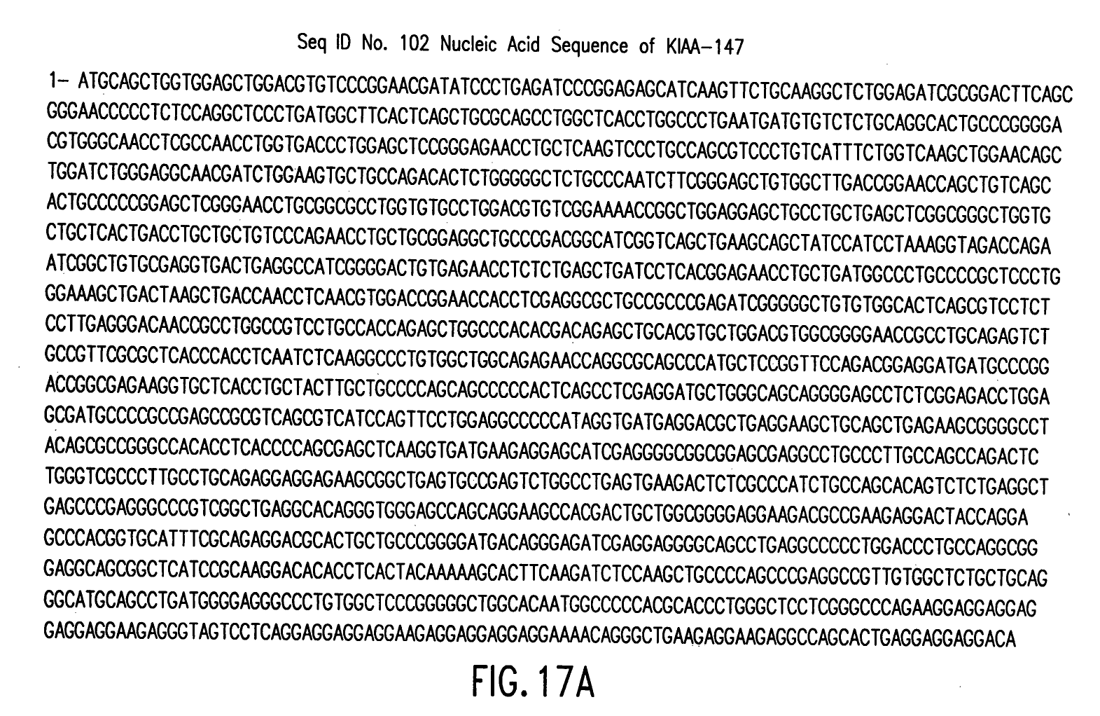

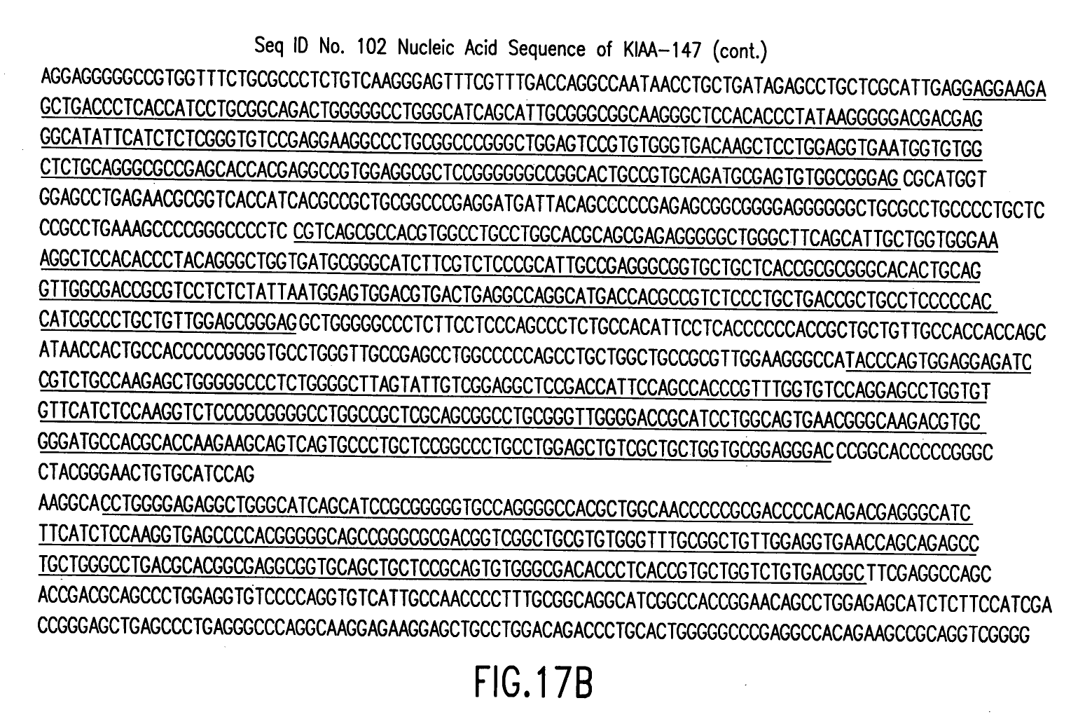

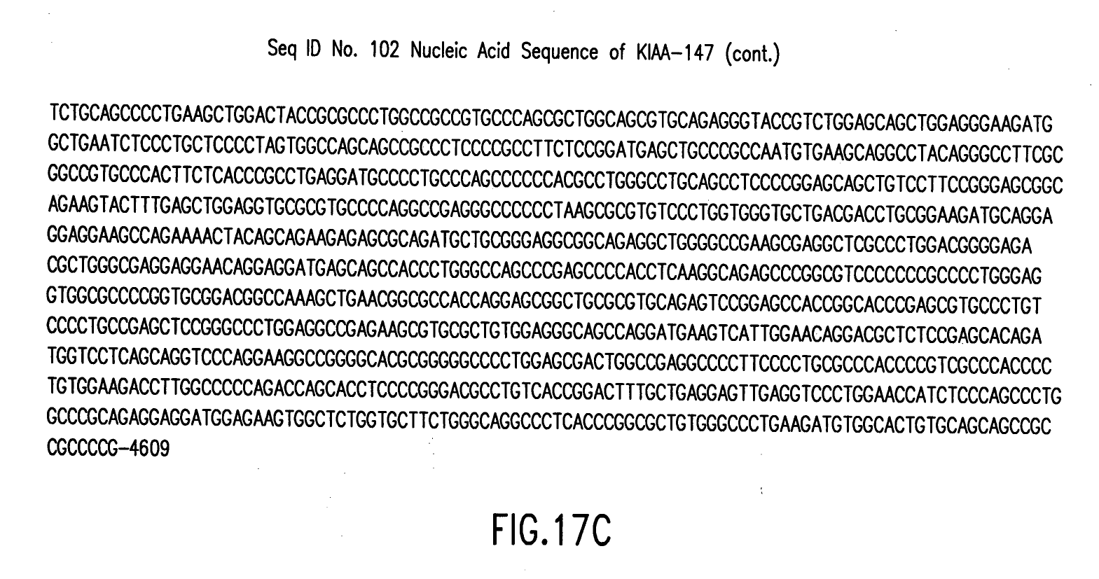

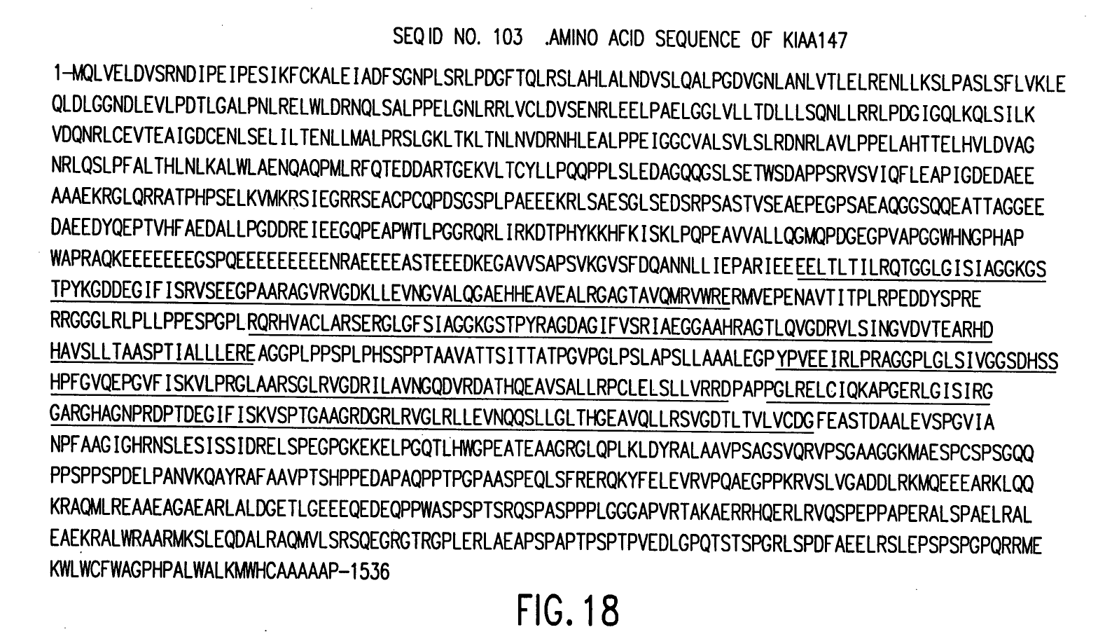

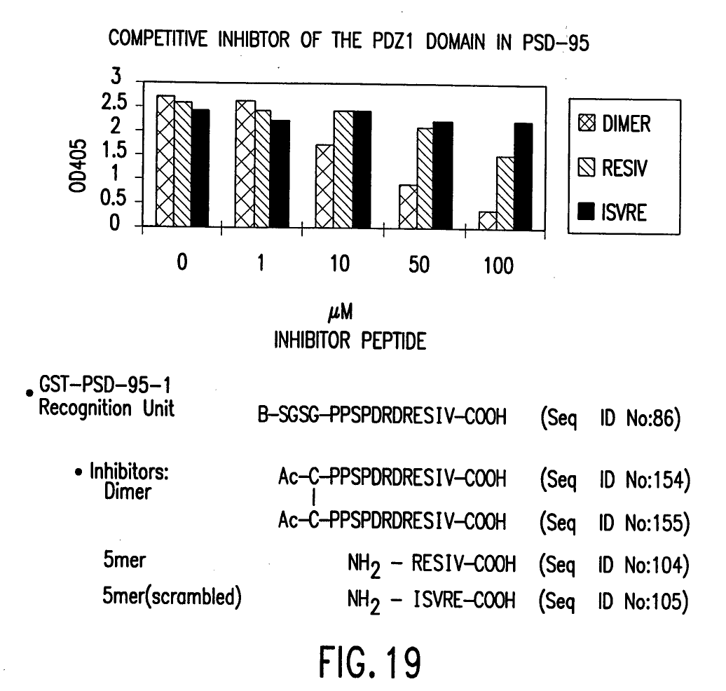

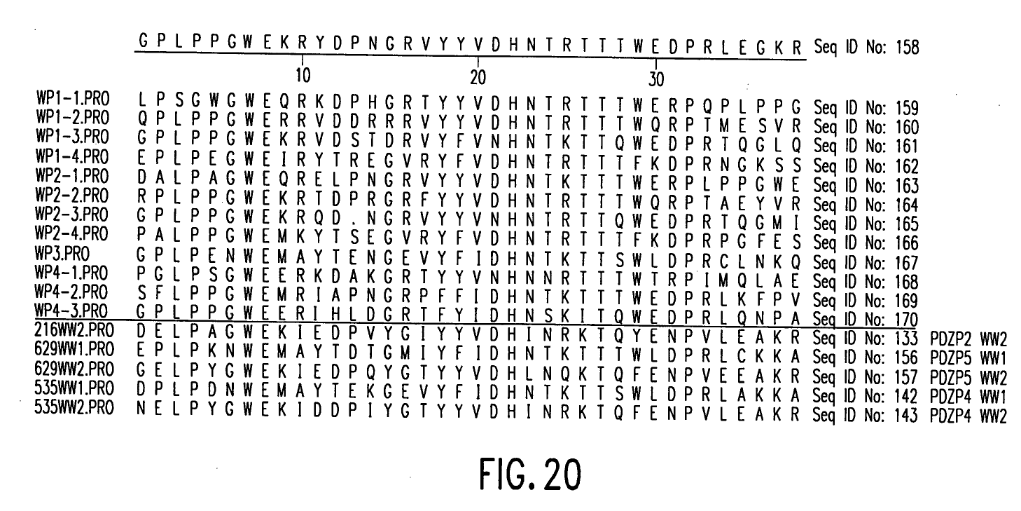

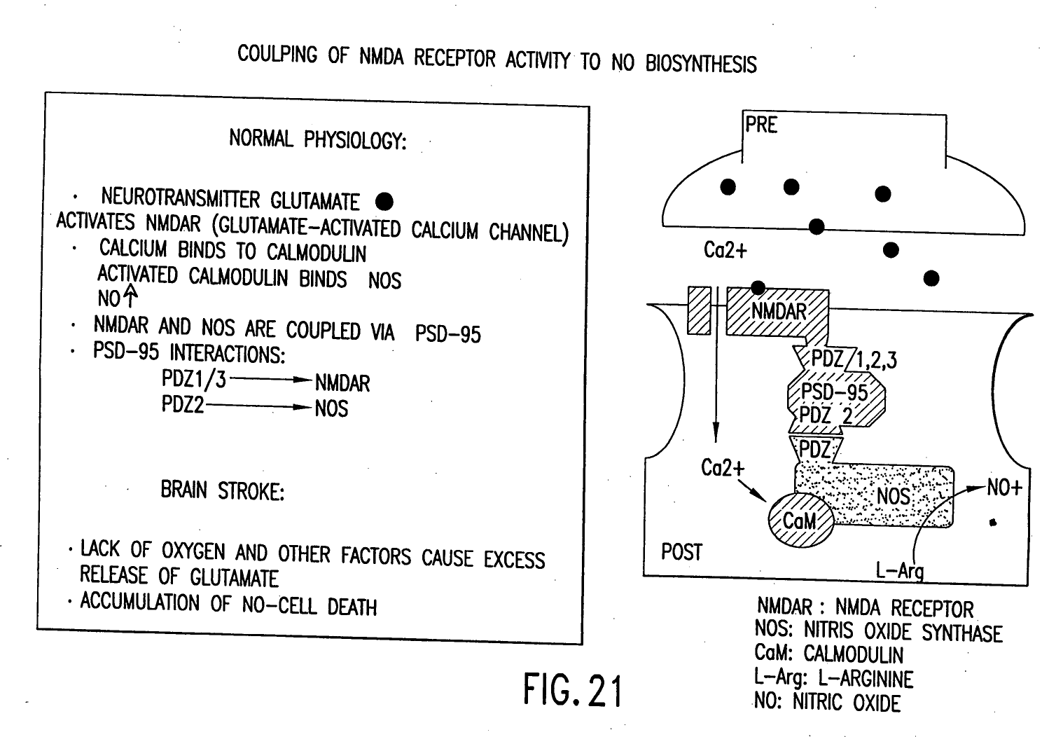

The present invention also provides a purified nucleic acid hybridizable to nucleotides 150-404 (SEQ ID NO:120), 579-866 (SEQ ID NO:121), 1077-1337 (SEQ ID NO:122), 1476-1732 (SEQ ID NO:123), 1914-2174 (SEQ ID NO:124), 2205-2461 (SEQ ID NO:125), 2613-2678 (SEQ ID NO:126), and/or 3006-3270 (SEQ ID NO:127) of the PDZP1 nucleotide sequence depicted in FIG. 7 (SEQ ID NO:75); nucleotides 359-655 (SEQ ID NO:128), 911-1156 (SEQ ID NO:129), 1421-1678 (SEQ ID NO:130), and/or 1892-2188 (SEQ ID NO:131) of the PDZP2 nucleotide sequence depicted in FIG. 9 (SEQ ID NO:77); nucleotides 118-377 (SEQ ID NO:134) and/or nucleotides 409-663 (SEQ ID NO:135) of the PDZP3 sequence depicted in FIG. 11 (SEQ ID NO:79); nucleotides 548-798 (SEQ ID NO:136), 1157-1396 (SEQ ID NO:137), 1634-1891 (SEQ ID NO:138), and/or 2063-2341 (SEQ ID NO:139) of the PDZP4 nucleotide sequence depicted in FIG. 13 (SEQ ID NO:98); nucleotides 742-999 (SEQ ID NO:144), 1246-1480 (SEQ ID NO:145), 1507-1947 (SEQ ID NO:146), and/or 2068-2337 (SEQ ID NO:147) of the PDZP5 nucleotide sequence depicted in FIG. 5 (SEQ ID NO:100); and nucleotides 1138-1397 (SEQ ID NO:152), 2329-2604 (SEQ ID NO:153), 2755-2925 (SEQ ID NO:154), and/or 2931-3270 (SEQ ID NO:155) of the KIAA-147 nucleotide sequence depicted in FIG. 17B (SEQ ID NO:102).

The present invention also provides antibodies to a polypeptide having an amino acid sequence selected from the group consisting of: SEQ ID NOS:10-27 and 111-116.

The present invention also provides antibodies to a polypeptide having an amino acid sequence selected from the group consisting of SEQ ID NOS:76, 78, 80, 99, 101.

It has been demonstrated by way of example herein that recognition units that comprise PDZ domain ligands derived from database searches for sequences having a homology to PDZ ligands may be used in the methods of the present invention as probes for the rapid discover of novel proteins containing functional PDZ domains. The methods of the present invention require no prior knowledge of the characteristics of a PDZ domain's natural cellular ligand to initiate the process of discovery, however, that knowledge can be used to expedite the process. In addition, because the methods of the present invention identify novel proteins from cDNA expression libraries based only on their binding properties, low primary sequence identity between the known PDZ domain and the PDZ domains of the novel proteins discovered need not be a limitation, provided some functional similarity between these PDZ domains is conserved. Also, the methods of the present invention are rapid, require inexpensive reagents, and employ simple and well established laboratory techniques.

Using these methods, thirteen different PDZ domain-containing proteins have been identified, of which five have not been previously described. Additionally, a known protein was found to contain four PDZ domains that were not previously recognized. These novel proteins are described more fully in Sections 6.1 and 6.2.

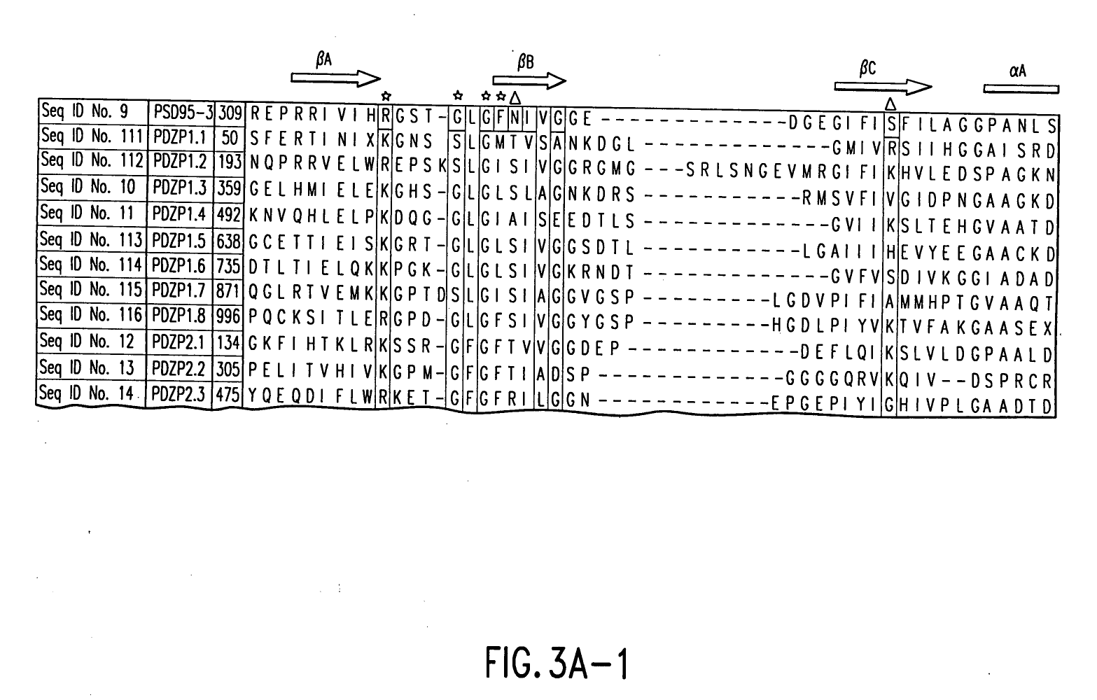

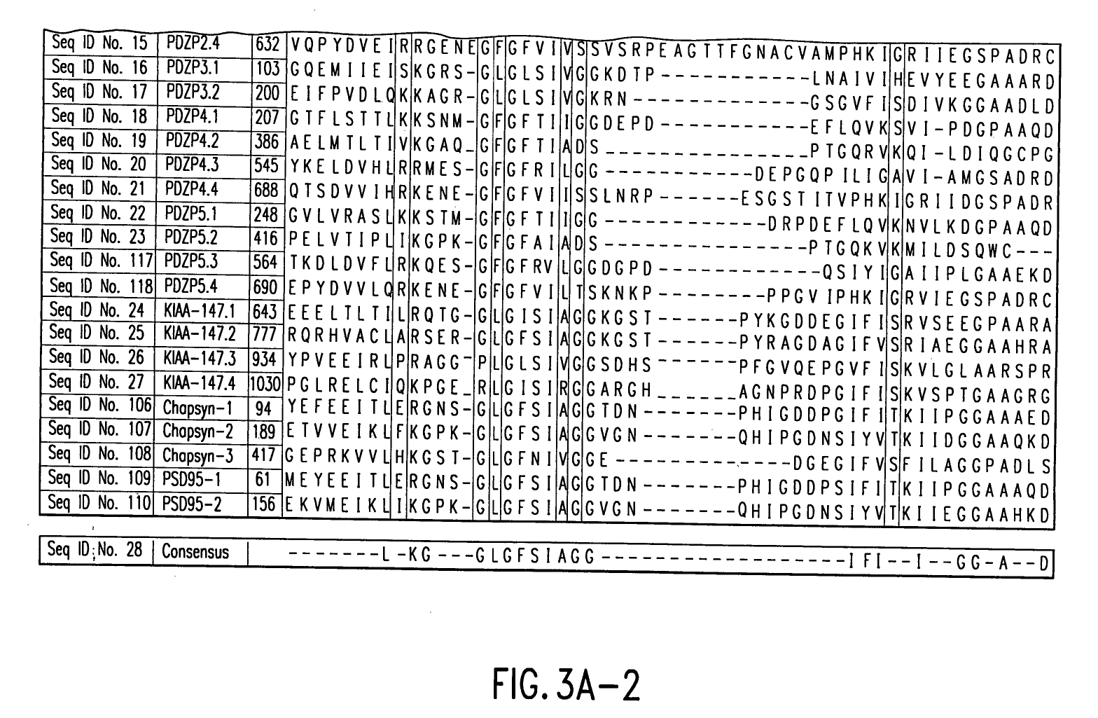

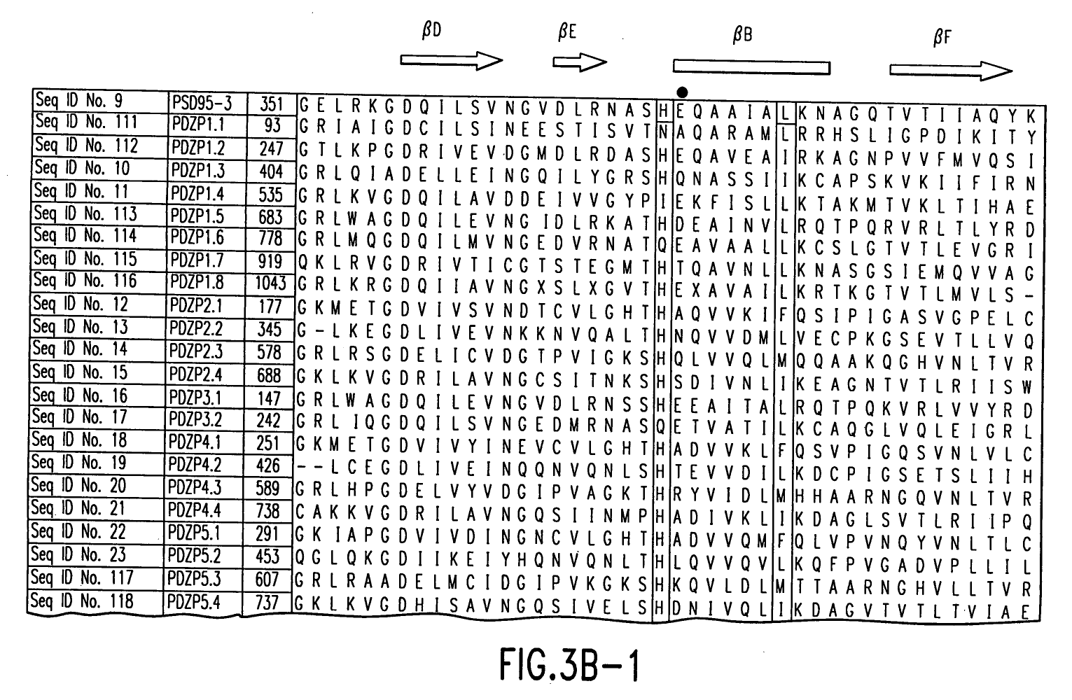

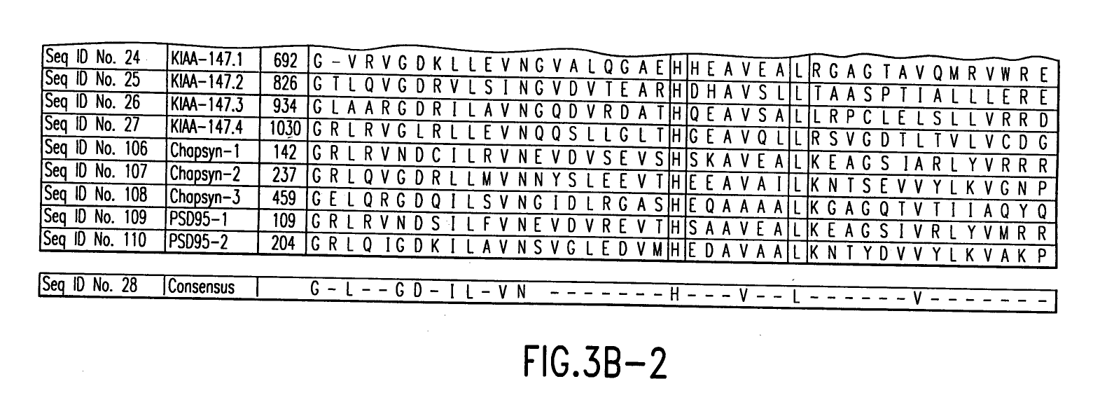

One of ordinary skill in the art would recognize that the above-described novel proteins need not be used in their entirety in the various applications of those proteins described herein. In many cases it will be sufficient to employ that portion of the novel protein that contains the PDZ domain. Such exemplary portions of PDZ domain-containing proteins are shown in FIGS. 3A and 3B. Accordingly, the present invention provides derivatives (e.g., fragments and molecules comprising these fragments) of novel proteins that contain PDZ domains, e.g., as shown in FIGS. 3A and 3B. Nucleic acids encoding these fragments or other derivatives are also provided.

### 5.1.1. PDZ Domains

PDZ domains in the practice of the present invention can take many forms and may perform a variety of functions. For example, such PDZ domains may be involved in a number of cellular, biochemical, or physiological processes, such as cellular signal transduction, cell-cell contacts, clustering of ion channels and receptors, transcriptional regulation, protein ubiquitination, cell adhesion, cytoskeletal organization, and the like. In particular embodiments of the present invention, the PDZ domains may be found in proteins including, but not limited to, PDZP1, PDZP2, PDZP3, PDZP4, PDZP5, PSD-95, chapsyn, KIAA, SAP-90, hdlg, NJRF, TKA-1, NMDAR, nNOs, EAP-1, LCAF/IL-16, Ina D, ZO-1, Z0-2, p55, bSYN1, bSYN2, PTP-BAS, PTPH1/PTP-MEG, LIMK, MAST-205, Tlam, Af-6, Dsh, LCAF, NK/T-ZIP, Ros-1, RO1/H 10.8, F28FS, F54E7, and LIN-Z/CASK. In another embodiment the PDZ domains may be found in proteins known to contain a PDZ domain, including but not limited to, those proteins enumerated in Table 3, or otherwise known in the art (see e.g., Ponting et al., 1997, Prot. Science 6:464-468, which is herein incorporated by reference in its entirety).

PDZ domains may be used for screening a random peptide library to identify peptides and their sequences reflective of the binding specificity of the particular PDZ domain. The peptides can then be formulated into recognition unit complexes for screening cDNA libraries to identify additional PDZ domains. The PDZ domains and recognition unit reagent pairs provide for an easily formatted assay whereby interference with the binding pairs by prospective drug molecules can be measured. As described in Section 5.3, cross affinity maps documenting the binding of various PDZ domains with various PDZ ligands are used in characterizing drug candidate's effects on the interactions, be it specific to one or one group of PDZ domain interactions or generic to most or all PDZ domain interactions. Specific embodiments of the invention are directed to 5 novel human proteins containing 20 novel PDZ domains and to the PDZ domains contained in these proteins. Other specific embodiments of the invention are directed to the 4 PDZ domains in a protein previously identified, but of unknown function.

TABLE 3
PROTEINS CONTAINING PDZ DOMAINS

| | | Number | | |
|---|---|---|---|---|
| | | of | | |
| | | PDZ | | EMBL |
| Protein | | domains | Species | codes^{a} |
| Membrane-Associated | | | | |
| Guanylate Kinases (MAGUKs) | | | | |
| Dlg (SAP97) | Discs-large | 3 | Human, rat, | U13896 |
| | tumour | | C. elegans | |
| | suppressor (dlg) | | | |
| Dlg-A | Discs-large | 3 | Drosophila | M73529 |
| | tumour | | | |
| | suppressor (dlg) | | | |
| SAP90 | Synapse- | | Rat | X66474 |
| | associated | | | |
| | protein Mᵣ 90,000 | | | |
| PSD95 | Postsynaptic | 3 | Mouse, rat, | D50621 |
| | density Mᵣ 95,000 | | human^{♯} | |
| ZO-1 | Zonula | 3 | Human, mouse, | L14837 |
| | occludentes | | C. elegans | |
| | protein-2 | | | |
| ZO-2 | Tamou gene | 3 | Human, dog | L27476 |
| | product (ZO- | | | |
| | 1/ZO-2-like) | | | |
| TamA | | 3 | Drosophila | D83477 |
| KAP-5/PSD- | K⁺-channel | 3 | Human, rat | U32376 |
| 93/ | associated | | | |
| Chapsyn- | protein | | | |
| 110 | (clone 5)/ | | | |
| | Channel- | | | |
| | associated | | | |
| | protein of | | | |
| | synapse-110 | | | |
| NE-Dlg/SAP- | Neuroendocrine | 3 | Human, mouse | U49089 |
| 102 | Dlg or SAP102 | | rat | |
| p55/MPP1 | Erythrocyte | 1 | Human, | M64925 |
| | membrane | | Fungurubripes, | |
| | protein, Mᵣ | | mouse | |
| | 55,000 | | | |
| Dlg-2 | Dlg-like protein 2 | 1 | Human | X82895 |
| Dlg-3 | Dlg-like protein 3 | 1 | Human | U37707 |
| Lin-2/CASK/ | Guanylate | 1 | C. elegans, | X92564 |
| CamGuk | kinases with N- | | rat, Drosophila | |
| | terminal | | | |
| | Cam kinase | | | |
| | domains | | | |
| Protein tyrosine | | | | |
| phosphatases (PTPS) | | | | |
| PTP-BAS | PTP with N- | 5 | Human, | U12128 |
| (FAP-1/ | terminal band | | bovine, | |
| PTP1E/BA14) | 4.1 domain | | mouse, | |
| | | | C. elegans | |
| PTP-MEG | Non-receptor | 1 | Human, | M68941 |
| | type 3 | | C. elegans | |
| | megakaryocyte | | | |
| | PTP | | | |
| PTP1H | Non-receptor | 1 | Human | M64572 |
| | type 3 PTP | | | |
| LIM-domain | | | | |
| containing | | | | |
| LIM kinase-1 | Serine/threonine | 1 | Human, rat, | D26309 |
| | kinase | | mouse | |
| LIM-kinase-2 | Serine/threonine | 1 | Human, rat, | D45906 |
| | kinase | | chicken | |
| Ril (rit- | Contains single | 1 | Rat, human | X76454 |
| 18) | LIM domain | | | |
| CLP36 | Contains single | 1 | Rat | U23769 |
| | LIM domain | | | |
| Enigma | Protein kinase | 1 | Human, rat | L35240 |
| | C-binding | | | |
| | protein | | | |
| ORF | | 1 | C. elegans | Z54237 |
| Other eukaryotic proteins | | | | |
| 9-PDZ | C. elegans ORF | 9 | C. elegans | Z46792 |
| containing | (C52a11.4) | | | |
| InaD^{b} | Inactivation-no- | 5 | Drosophila, | U15803 |
| | after-potential D | | C. vicina | |
| Rhophilin | GTP-Rho-binding | 1 | Mouse | U43194 |
| | protein | | | |
| LR Repeat | ORF (Kiaa0147) | 4 | Human^{♯} | D63481 |
| ORF | containing Leu- | | | |
| | rich repeats | | | |
| AF-6/canoe | Ras-binding | 1 | Human, | U02478 |
| | protein | | Drosophila | |
| Syntrophins | Dystrophin- | 1 | Human, mouse, | U40571 |
| | associated | | T. californica, | |
| | proteins (≧3 | | C. elegans, | |
| | isoforms) | | rabbit | |
| Dsh/Dvl | dishevelled gene | 1 | Drosophila, | U46461 |
| | products (≧3 | | human, mouse, | |
| | isoforms) | | frog, | |
| | | | C. elegans | |
| X11 | Gene expressed | 2 | Human, mouse | L04953 |
| | in nervous | | | |
| | system | | | |
| Rabphilin- | Synaptic vesicle | 1 | C. elegans | U41035 |
| like | trafficking | | | |
| | protein? | | | |
| PAR-3^{b} | Role in | 3 | C. elegans | U25032 |
| | establishing | | | |
| | polarity in | | | |
| | embryos | | | |
| Leu-Zipper | Putative | 1 | Human | L06633 |
| | transcription | | | |
| | protein | | | |
| TKA-1 | Tyrosine kinase | 2 | Human | Z50150 |
| | activator 1 | | | |
| PKA | Protein kinase A | 2 | Rabbit, mouse | U19815 |
| cofactor | regulator | | | |
| nNOS | Neuronal nitric | 1 | Human, mouse, | U17327 |
| | oxide synthase | | rat | |
| MAST205 | Microtubule- | 1 | Mouse | U02313 |
| kinase | associated S/T | | | |
| | kinase (205 kDa) | | | |
| PICK-1 | Protein that | 1 | Mouse | Z46720 |
| | interacts with | | | |
| | C-kinase 1 | | | |
| IL-16 (LCF) | Interleukin-16 | 2 | Human, | S81601 |
| | | | C. aethiops | |
| GAP | BCR-like GTPase | 1 | C. elegans | U28741 |
| | activator | | | |
| | protein | | | |
| Tiam-1 | T-lymphocyte | 1 | Mouse, human | U16296 |
| | invasion and | | | |
| | metastasis | | | |
| Still life | May regulate | 1 | Drosophila | D86547 |
| (slf) | synaptic | | | |
| | differentiation | | | |
| Densin-180 | Brain-specific | 1 | Rat | U66707 |
| | PSD protein | | | |
| APX-like | Human | 1 | Human | X83543 |
| | homologue of | | | |
| | frog APX gene | | | |
| Periaxin | Protein of | 1 | Rat, C. elegans | Z29649 |
| | myelinating | | | |
| | Schwann cells | | | |
| ros1′ | Gene product | 1 | Human | g226930 |
| | aberrantly fused | | | |
| | to ros1 | | | |
| Lin-7 | Cell junction | 1 | C. elegans | U78092 |
| | protein | | | |
| Spa-1 | GTPase activator | 1 | mouse | D11374 |
| | protein for | | | |
| | Rap1, Rsr1, Ran | | | |
| LT-antigen | Viscerotropic | 1 | L. tropica | U31221 |
| | leishmaniasis | | | |
| | antigen | | | |
| ORF | Contains SAM | 1 | C. elegans | Z48367 |
| (SAM/ANK) | and ankyrin | | | |
| | domains | | | |
| | (C33b4.3) | | | |
| ORF (SAM) | ORF containing | 1 | C. elegans | Z31590 |
| | SAM domain | | | |
| | (R01h10.8) | | | |
| ORF (SAM) | ORF containing | 1 | C. elegans | U80437 |
| | SAM domain | | | |
| | (C43e11.6) | | | |
| ORF (PX) | ORF containing | 1 | C. elegans | Z79754 |
| | PX domain | | | |
| | (F25h2.2) | | | |
| ORF (C2) | ORF containing | 1 | C. elegans | U70852 |
| | C2 domain | | | |
| | (F45e4.3) | | | |
| 11 C. elegans | C53b4.4, | 1 | C. elegans | ^{c} |
| ORFs | F28f5.3, | | | |
| | C01f6.6, | | | |
| | C45g9.7, | | | |
| | T21c9.1, | | | |
| | C52a11.3, | | | |
| | C01b7.5, | | | |
| | F20d6.1, | | | |
| | C25g4.6, | | | |
| | C35d10.2, | | | |
| | F44d12.4 | | | |
| Bacterial PDZ-containing | | | | |
| proteins (and their | | | | |
| eukaryotic homologues) | | | | |
| hrtA | High-temperature | 2 | E. coli etc. | M36536 |
| | requirement A | | | |
| hhoA | hrtA-like | 2 | E. coli etc. | U15661 |
| | protein | | | |
| hhoB | hrtA-like | 1 | E. coli etc. | U15661 |
| | protein | | | |
| N1897 | Protein with 2 | 4 | S. cerevisiae | Z71399 |
| | tandem hrtA-like | | | |
| | repeats | | | |
| hrtA(IGFB) | htra- and IGF- | 1 | Human | Y07921 |
| | binding protein- | | | |
| | like regions | | | |
| spoIVB | Stage IV | 1 | B. subtilis, | M30297 |
| | sporulation | | C. difficle | |
| | protein B | | | |
| Yael | Hypothetical | 1 | E. coli, | D83536 |
| | protein | | H. influenzae | |
| OFR | Hypothetical | 1 | Anabaena sp., | U21853 |
| | protein | | Synechocystis | |
| | | | sp. | |
| ^{♯} Fragment | | | | |
| ^{a}EMBL accession codes are shown for the first-mentioned species | | | | |
| # (literature citations may be found in these database entries). | | | | |

^{b}The number of PDZ domains predicted by this analysis differs from that in previous publications.
^{c}Z68215, U00045, Z68213, U21323, Z73098, Z46792, U53147, U50301, Z70680, U21324, Z68298.

TABLE 4

| | | |
|---|---|---|
| Seq ID No. 111 | PDZP1.1 | SFERTTNIXKGNSSLGMTVSANKDGLGMIVRSIIHGGAISRDGRIAIGDCILSINESSTISVTNAQARAMLRRHSL |
| | | IGPDIKITY |
| Seq ID No. 112 | PDZP1.2 | NQPRRVELWREPSKSLGISIVGGRGMGSRLSNGEVMRGIFIKFIVLEDSPAGKNGTLKPGDRIVEVDGMDLRDASH |
| | | EQAVEARIKAGNPVVFMVQSI |
| Seq ID No. 10 | PDZP1.3 | GELHMIELEKGHSGLGLSLAGNKDRSRMSVEIVDPNGAAGKDGRLQIADELLEINGQILYGRSHQNASSIIKCAPS |
| | | KVKIIEIRNK |
| Seq ID No. 11 | PDZP1.4 | KNVQHLELPKDQGGLGIASEEDTLSGVTIKSLTEHGVAATDGRLKVGDQILAVDDEIVVGYPIEKFISLLKTAKMT |
| | | VKLTIHAE |
| Seq ID No. 113 | PDZP1.5 | GCETTIEISKGRTGLGLSIVGGSDTLLGAIIIHEVYEEGAACKDGRLWAGDQILEVNGIDLRKATHDEAINVLRQT |
| | | PQRVRLTLYRD |
| Seq ID No. 114 | PDZP1.6 | DTLTIELQKKPGKGLGLSIVGKRNDTGVFVSDIVKGGIADADGRLMQGDQILMVNGEDVRNATQEAVAALLKCSLG |
| | | TVTLEVGRI |
| Seq ID No. 115 | PDZP1.7 | QGLRTVEMKKGPTDSLGISIAGGVGSPLGDVPIFIAMMHPTGVAAQTQKLRVGDRIVTICGTSTEGMTHTQAVNLL |
| | | KNASGSIEMQVVAG |
| Seq ID No. 116 | PDZP1.8 | PQCKSITLERGPDGLGFSIVGGYGSPHGDLPIYVKTVFAKGAASEXGRLKRGDQIIAVNGXSLXGVTHEXAVAILK |
| | | RTKGTVTLMVLSIGCXN |
| Seq ID No. 12 | PDZP2.1 | GKFIHTKLRKSSRGEGFTVVGGDEPDEELQIKSLVLDGPAALDGKMETGDVIVSVNDTCVLGIITIIAQVVKIFQS |
| | | IPIGASVGPELC |
| Seq ID No. 13 | PDZP2.2 | PELITVHIVKGPMOFGFTIADSPGGGGQRVKQIVDSPRCRGLKEGDLIVEVNKKNVQALTHNQVVDMLVECPKGSE |
| | | VTLLVG |
| Seq ID No. 14 | PDZP2.3 | YQEQDIFLWRKETGFGFRILGGNEPGEPIYIGHIVPLGAADTDGRLRSGDELICVDGTPVIGKSHQLVVQLMQQAA |
| | | KQGHVNLTV |
| Seq ID No. 15 | PDZP2.4 | QPYDVEIRRGENEGFGFVIVSSVSRPEAGTTFGNACVAMPHKIGRIIEGSPADRGGKLKVGDRILAVNGCSITNKS |
| | | IISDIVNLIKEAGNTVTLRIISW |
| Seq ID No. 16 | PDZP3.1 | GQEMIIEISKGRSGLGLS1VGGKDTPLNAIVIHEVYEEGAAARDGRLWAGQILEVNGVDLRNSSHIBEAITALRQT |
| | | PQKVRLVVYR |
| Seq ID No. 17 | PDZP3.2 | EIFPVDLQKKAGRGLGLSIVGKRNGSGVFISDIVKGGAADLDGRLIQGDQILSVNGEDMRNASQETVATILKCAQG |
| | | LVQLEIGRL |
| Seq ID No. 18 | PDZP4.1 | GTFLSYFLKKSNMGFGFTIIGGDEPDEFLQVKSVIPDGPAAQDGKMETGDVIVYINEVCVLGHTHADVVKLFQSVP |
| | | IGQSVNLVLC |
| Seq ID No. 19 | PDZP4.2 | AELMTLTIVKGAQGFGETIADSPTGQRVKQILDIQGCPGLCEGDLIVEINQQNVQNLSHTEVVDILKDCPIGSETS |
| | | LIHH |
| Seq ID No. 20 | PDZP4.3 | YKELDVIILRRMESGFGFRILGGDEPGQPILIGAVIAMGSADRDGRLHPGDELVVVDGIPVAGKTIIRYVIDLMHH |
| | | AARNGQVNLTVR |
| Seq ID No. 21 | PDZP4.4 | QTSDVVIHRKENEGFGFVIISSLNRPESGSTITVPHKIGRIIDGSPADRCAKLKVGDRILAVNGQSIINMPHADIV |
| | | KLIKDAGLSVTLRIIPQ |
| Seq ID No. 22 | PDZP5.1 | GVLVRASLKKSTMGFGFTIIGGDRPDEFLQVKNVLKDGPAAQDGKIAPGDVIVDINGNCVLGHTHADVVQMFQLVP |
| | | VNQYVNLTLC |
| Seq ID No. 23 | PDZP5.2 | PeLVTIPLIKGPKGFGFAIADSPTGQKVKMILDSQWCQGLQKGDIIKEIYHQNVQNLTHLQVVQVLKQFPVGADVP |
| | | LLIL |
| Seq ID No. 117 | PDZP5.3 | TKDLDVFLRKQESGFGFRVLGGDGPDQSIYIGAIIPLGGAEKDGRLRAADELMCIDGIPVKGKSHKQVLDLMTTAA |
| | | RNGHVLLTVR |
| Seq ID No. 118 | PDZP5.4 | EPYDVVLQRKENEGFGFVILTSKNKPPPGVIPHKIGRVIEGSPADRCGKLKVGDIIISAVNGQSIVELSHDNIVQL |
| | | IKDAGVTVTLTVIAE |
| Seq ID No. 82 | KIAA147.1 | EELTLTILRQTGGLGISIAGGKGSTPYKGDDEGIFISRVSEEGPAARAGVRVGDKLLEVNGVALQGAEHHEAVEAL |
| | | RGAGTAVQMRVWRE |
| Seq ID No. 83 | KIAA147.2 | RQRHVACLARSERGLGFSIAGGKGSTPYRACDAGIFVSRIAEGGAAHRAGTLQVGDRVLSINGVDVTEARHDHAVS |
| | | LLTAASPTIALLLERE |
| Seq ID No. 84 | KIAA147.3 | YPVEEIRLPRAGGPLGLSIVGGSDhSSHPFGVQEPGVFISKVLPRGLAARSGLRVGDRILAVNGQDVRDATHQFAV |
| | | SALLRPCLELSLLVRRD |
| Seq ID No. 85 | KIAA147.4 | PGLRELCIQKAPGERLGISIRGGARGHAGNPRDPTDEGIFTSKVSPTGAAGRDGRLRVGLRLLEVNQQSLLLGLTH |
| | | GEAVQLLRSVGDTLTVLVCDG |
| Seq ID No. | Consensus: | GXLYX₂GDXILXVNX₈HX₃VX₂LX₆VX₁₄LXXGX₃₋₄GLGfSIAGGX₄₋₁₈IFIX₂IX₂GGXAX₂D |

In one embodiment of the invention, a suitable target molecule containing a PDZ domain is selected. A number of proteins may be selected as the target molecule, including but not limited to: PDZP1, PDZP2, PDZP3, PDZP4, PDZP5, PSD-95, Chapsyn, KIAA, SAP-90, hdlg, NJRF, TKA-1, NMDAR, nNOS, EAP-1, LCAF/IL-16, Ina D, ZO-1, Z0-2, p55, bSYN1, bSYN2, PTP-BAS, PTPH1/PTP-MEG, LIMK, MAST-205, Tlam, Af-6, Dsh, LCAF, NK/T-ZIP, Ros-1, RO1/H 10.8, F28FS, F54E7, and LIN-Z/CASK. Alternatively, the target molecule may be any of the proteins enumerated in Table 3 or otherwise known in the art. Alternatively, a portion of the above-mentioned proteins comprising the PDZ domain may be chosen as the target molecule.

### 5.1.2. Recognition Units

By the phrase “recognition unit,” is meant any molecule having a selective binding affinity for the PDZ domain of the target molecule and, preferably, having a molecular weight of up to about 40,000 daltons. In a particular embodiment of the invention, the recognition unit has a molecular weight that ranges from about 100 to about 10,000 daltons.

Accordingly, preferred recognition units of the present invention possess a molecular weight of about 100 to about 5,000 daltons, preferably from about 100 to about 2,000 daltons, and most preferably from about 500 to about 1,500 daltons. As described further below, a recognition unit of the present invention can be a peptide, a carbohydrate, a nucleoside, an oligonucleotide, any small synthetic molecule, or a natural product. When the recognition unit is a peptide, the peptide preferably contains about 4 to about 150 amino acid residues. Since PDZ domains have been observed to bind with other PDZ domains, the recognition units of the invention may be polypeptides containing a PDZ domain, these polypeptides may be greater than 50 amino acid residues; preferably the peptide has greater than 80, 90, 100, 110, or 150 amino acid residues.

In other embodiments, the recognition unit is a peptide containing less than about 100 amino acid residues; preferably, the peptide has less than about 80 amino acid residues; preferably, the peptide has less than about 70 amino acid residues; preferably, the peptide has 4 to 30 amino acid residues; most preferably, the peptide has about 4 to 15 amino acid residues.

The step of choosing a recognition unit peptide can be accomplished in a number of ways, including but not limited to, database searches for molecules having homology with known ligands having the ability to selectively bind to a PDZ domain. In specific embodiments of the invention, databases are screened for stretches of amino acid sequences comprising the sequence Xaa-(Ser/Thr)-Xaa-Val-COOH (SEQ ID NO:4) or Xaa-(Ser/Thr)-Xaa-Yaa (SEQ ID NO:82), where Xaa can be any amino acid and Yaa is a small hydrophobic amino acid. In preferred embodiments, these amino acid sequences are located at the carboxyl terminus of the polypeptide. In specific embodiments, the recognition units used according to the methods of the invention are proteins, derivatives (including fragments) or analogs of proteins selected from the group consisting of: protein name (H, P18090); Serotonin Receptor (H, P28223); VIP Receptor (H, P32241); CRF Receptor (H, P34998); Orphan Receptor (H, P46089); β-1 Adrenergic Receptor (H, P08588); COM (ADE02, P03267); E6, HPV18 (V, P06463); UL25, HSV11 (V, P10209); GP3, EBV (V, P03200); TAT, HTL1A (V, P03409); UL14, VZVD (V, P09295); NMDA Receptor, NR2B (M, Q01097); NMDA Receptor subunit (H, U08266); mGluR1α (H, U31215); mGluR5a (H, D28538); mGluR (H, L76631); mGluR3 (H, AC002081); AMPA receptor (H, L20814); K⁺-Channel, KV 1.4 (H, P22459); K⁺-Channel Kir 2.2v (H, U53143); Na⁺-Channel (α) (H, P15389); K⁺-Channel (Kir) (H, D50582) ; Transmembrane Receptor (Homolog of frizzled ) (H, U43318); Homolog of frizzled (R, L02529); Homolog of frizzled (M, U43319); Glucose transporter (H, P11166); Excitatory Amino Acid Transporter (H, P43003); FAS Receptor (H, P25445); NGF Receptor (H, P08138); Neuropeptide Y Receptor, type 2 (H, P49146); Somatostatin Receptor, type 2 (H, P30874); CFTR (H, P13569); V-CAM (H, P19320); Ankyrin (H, Q01484); Fanconi anemia group C protein (H , Q00597); Calcium pump (H, P23634); APC protein (H, P25054); BCR, (H, P11274); MPK2 (H, P36507); Colorectal Mutant Cancer Protein (H, P23508); 65 KD Yes-Associated Protein (H, P46937); Neutrophil Cytosol Factor 1 (H, P14598); Neurexin III, (B, L27869); Neurexin II (B, L14855). See FIGS. 5A and 5B.

The recognition unit can also be identified for use by screening cDNA libraries or random peptide libraries for a peptide that binds to a known PDZ domain. Essentially, screening cDNA libraries or random peptide libraries for a peptide that binds to a PDZ domain can be accomplished in the same manner as for screening cDNA libraries or random peptide libraries for a peptide that binds to an SH3 domain. See, e.g., Yu et al., 1994, Cell 76:933-945; Sparks et al., 1994, J. Biol. Chem. 269:23853-23856; Sparks et al., 1996, Proc. Natl. Acad. Sci. USA 93:1540-1544 for screening of peptide libraries to discover peptides that bind to SH3 domains.

Alternatively, a small molecule or drug may be known to those of ordinary skill to bind to a certain target molecule containing a PDZ domain. The recognition unit can even be synthesized from a lead compound, which again may be a peptide, carbohydrate, oligonucleotide, small drug molecule, or the like.

In a specific embodiment, the step of selecting a recognition unit for use can be effected by, e.g., the use of diversity libraries, such as random or combinatorial peptide or nonpeptide libraries which can be screened for molecules that specifically bind to PDZ domains. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37:1233-1251; Ohimeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian et al., 1992, J. Mol. Biol. 227:711-718); Lenstra, 1992, J. Immunol. Meth. 152:149-157; Kay et al., 1993, Gene 128:59-65; and PCT International Publication WO 94/18318, published Aug. 18, 1994.

In vitro translation-based libraries include but are not limited to those described in International PCT Publication WO 91/05058, published Apr. 18, 1991; and Mattheakis et al., 1994, Proc. Natl. Acad. Sci. USA 91:9022-9026.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

The variety of non-peptide libraries that are useful in the present invention is great. For example, Ecker and Crooke (1995, Bio/Technology 13:351-360) list benzodiazepines, hydantoins, piperazinediones, biphenyls, sugar analogs, β-mercaptoketones, arylacetic acids, acylpiperidines, benzopyrans, cubanes, xanthines, aminimides, and oxazolones as among the chemical species that form the basis of various libraries.

Non-peptide libraries can be classified broadly into two types: decorated monomers and oligomers. Decorated monomer libraries employ a relatively simple scaffold structure upon which a variety functional groups is added. Often the scaffold will be a molecule with a known useful pharmacological activity. For example, the scaffold might be the benzodiazepine structure.

Non-peptide oligomer libraries utilize a large number of monomers that are assembled together in a ways that create new shapes that depend on the order of the monomers. Among the monomer units that have been used are carbamates, pyrrolinones, and morpholinos. Peptoids, peptide-like oligomers in which the side chain is attached to the α amino group rather than the α carbon, form the basis of another version of non-peptide oligomer libraries. The first non-peptide oligomer libraries utilized a single type of monomer and thus contained a repeating backbone. Recent libraries have utilized more than one monomer, giving the libraries added flexibility.

In a preferred embodiment, knowledge of PDZ domain binding to C-terminal ends of proteins is used in selecting libraries to screen for PDZ binding ligands that are then used in recognition unit complexes to further search for PDZ domain containing proteins. In the oriented peptide library approach (Songyang et al., 1993, Cell 72:767-778) a soluble mixture of peptides represented by the formula Lys-Asn-Xaa₆-COOH (SEQ ID NO: ______), or Lys-Asn-Xaa₆-(Ser/Thr/Tyr)-Xaa₂-COOH (SEQ ID NO: ______) (where Xaa is any amino acid except Cys and Trp), are passed over a column containing the protein domain and the subgroup of peptides retained by the column is sequenced to obtain a consensus sequence.

Alternatively, a random peptide library constructed as described by Schatz et al. (1996, Methods Enzymol 267:171-191) can be used. For example, Striker (1997, Nat. Biotechnology 15:336-342) used a random 15mer library constructed using oligonucleotides with degenerate regions of codons in the form of NNK (N=A,G,T, and C; K=G and T nucleotide bases). Examples of peptides identified by these strategies that may be used as recognition unit complexes for screening for PDZ domains are described in Songyang et al., (1993, Cell 72:767-778); Striker (1997, Nat. Biotechnology 15:336-342); and Kornau, 1995, Science 269:1737-1740.

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992, BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT International Publication WO 94/18318, published Apr. 18, 1994.

In a specific embodiment, screening to identify a recognition unit can be carried out by contacting the library members with a PDZ domain immobilized on a solid phase and harvesting those library members that bind to the PDZ domain. Examples of such screening methods, termed “panning” techniques are described by way of example in Parmley and Smith, 1988, Gene 73:305-318; Fowlkes et al., 1992, BioTechniques 13:422-427; PCT International Publication WO 94/18318, published Aug. 18, 1994; and in references cited hereinabove.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify recognition units that specifically bind to PDZ domains.

Where the recognition unit is a peptide, the peptide can be conveniently selected from any peptide library, including random peptide libraries, combinatorial peptide libraries, or biased peptide libraries. The term “biased” is used herein to mean that the method of generating the library is manipulated so as to restrict one or more parameters that govern the diversity of the resulting collection of molecules, in this case peptides.

Thus, a truly random peptide library would generate a collection of peptides in which the probability of finding a particular amino acid at a given position of the peptide is the same for all 20 amino acids. A bias can be introduced into the library, however, by specifying, for example, that a lysine occur every fifth amino acid or that positions 4, 8, and 9 of a decapeptide library be fixed to include only arginine. Clearly, many types of biases can be contemplated, and the present invention is not restricted to any particular bias. In preferred embodiments the peptide library is biased so as to favor peptides containing at their C-terminus the PDZ ligand consensus motif Xaa-Ser/Thr-Xaa-Val-COOH (SEQ ID NO:4) or the more general motif Xaa-Ser/Thr-Xaa-Yaa-COOH (SEQ ID NO:82), where Xaa can be any amino acid and Yaa is a small hydrophobic amino acid. Furthermore, the present invention contemplates specific types of peptide libraries, such as phage displayed peptide libraries and those that utilize a DNA construct comprising a lambda phage vector with a DNA insert.

As mentioned above, in the case of a recognition unit which comprises a peptide ligand of a PDZ domain, the peptide may have about 4 to less than about 80 amino acid residues, about 4 to less than 50 amino acid residues, preferably about 4 to about 30 amino acid residues, and most preferably, about 4 to about 15 amino acids. In another embodiment, a peptide recognition unit has in the range of 4-200 amino acids, 4-150 amino acids, 4-100 amino acids, or 4-50 amino acids.

The selected recognition unit can be obtained by chemical synthesis or recombinant expression. Chemical synthesis may be accomplished using techniques known in the art.

By example, and not by way of limitation, peptides may be synthesized using a variation of standard solid phase Fmoc peptide chemistry (Knorr et al., 1989, Tetrahedron Lett. 30:1927-1930) on standard support resins, including but not limited to, polystyrene or TentaGel® (Tübingen, Germany). Product yield can be increased by varying DMSO (dimethylsulfoxide) solvent mixtures used in the synthesis. Specifically proline rich regions require the use of 50% DMSO as a co-solvent with DMF (N,N-dimethylformamide) or NMP (N-methylpyrralidone) in order to obtain reasonable yields. Additionally, with respect to biotinylation, biotin is only marginally soluble in neat DMF or NMP, so this reagent was dissolved in DMSO and then diluted to 50% in NMP or DMF before coupling. Further, depending on the particular ligand, biotin sometimes requires a spacer moiety between it and the ligand.

The selected recognition units, whether obtained by chemical synthesis or recombinant expression, are preferably purified prior to use in screening a plurality of gene sequences.

A particular recognition unit may have fairly generic selectivity for several members (e.g., three or four or more) of a “family” of polypeptides having a PDZ domain (the same PDZ domain or different versions of a PDZ domain or functional equivalents of a PDZ domain of interest) or a fairly specific selectivity for only one or two, or possibly three, of the polypeptides among a “panel” of same. Furthermore, multiple recognition units, each exhibiting a range of selectivities among a “panel” of polypeptides can be used to identify an increasingly comprehensive set of additional polypeptides that include a PDZ domain.

Hence, in a population of related polypeptides, specificity of the PDZ domains of each member may be schematically represented by a circle. See, by way of example, FIG. 4A. The circle of one polypeptide may overlap with that of another polypeptide. Such overlaps may be few or numerous for each polypeptide. A particular recognition unit A, is specific for a group of PDZ domain containing polypeptides represented by circle A. Recognition unit B, on the other hand, has a broader specificity for PDZ domains represented by circles 1, 2, and 3. Subsets of PDZ domains of the B group show affinity also for recognition units B₁, B₂, B₃ and A. Recognition units B₁, B₂, and B₃ can now be used to screen for another group of PDZ domain containing proteins represented by circles 4, 5, and 6. PDZ domains represented by circle 4 also show affinity for ligands B₄ and B₅. B₄ and B₅ are now used to screen further identifying PDZ domain proteins represented by circles 7, 8, etc.

Hence, with a given recognition unit, one may observe interaction with only one or two different polypeptides. With other recognition units, one may find three, four, or more selective interactions. In the situation in which only a single interaction is observed, it is likely, though not mandatory, that the selective affinity interaction is between the recognition unit and a replica of the initial target molecule (or a molecule very similar structurally and “functionally” to the initial target molecule).

It should also be apparent to those of ordinary skill that any number of B-type recognition units (B₁, B₂, B₃, etc.) can be present, each recognizing different subfamilies of polypeptides. Hence, the use of multiple recognition units provides an increasingly more exhaustive population of polypeptides, each of which exhibits a variation or evolution in the PDZ domain present in the initial molecule. It should also be apparent to those of ordinary skill that the present method can be applied in an iterative fashion, such that the identification of a particular polypeptide can lead to the choice of another recognition unit. See, e.g., FIG. 4B. Use of this new recognition unit will lead, in turn, to the identification of other polypeptides that contain PDZ domains that enhance the phenotypic and/or genotypic diversity of the population of “related” polypeptides.

As discussed above, in one embodiment, recognition units are obtained by database searches for recognition units with sequence homology to known recognition units. In other embodiments, a source of recognition units, e.g., a cDNA expression library or a phage display library, may be screened for recognition units that bind to a particular target PDZ domain. In an additional embodiment, if a recognition unit for a particular target PDZ domain is already known, there is no need to screen a library or other source of recognition units; one can merely synthesize that particular recognition unit.

The recognition unit, however obtained, is then used to screen an expression library or other source of polypeptides to identify polypeptides that the recognition unit binds to. A recognition unit that identifies only its target PDZ domain is a recognition unit that is completely specific. A recognition unit that identifies one or two other polypeptides that do not contain identically the target PDZ domain, from among a plurality of polypeptides (e.g., of greater than 10⁴, 10⁶, or 10⁸ complexity), in addition to identifying a molecule comprising its target PDZ domain, is very or highly specific. A recognition unit that identifies most other polypeptides present that do not contain its target PDZ domain, in addition to identifying its target PDZ domain, is non-specific. In between very specific recognition units and non-specific probes, the present inventors have discovered that there are recognition units that recognize a small number of molecules having PDZ domains other than their target PDZ domains. These recognition units are said to have generic specificity.

Thus, there is a “specificity continuum”, from completely and very specific through generic to non-specific, that a recognition unit may evince. Thus, the degree of specificity or selective affinities observed among the recognition units of the invention varies widely, generally falling in the range of about 1 nm to about 1 mM. In preferred embodiments of the present invention, the selective affinity falls on the order of about 10 nM to about 100 μM, more preferably on the order of about 100 nM to about 10 μM, and most preferably on the order of about 100 nM to about 1 μM.

Usually, high specificity is considered to be desirable when screening a library. High specificity is exhibited, e.g., by affinity purified polyclonal antisera which, in general, are very specific. Monoclonal antibodies are also very specific. Small peptides in monovalent form, on the other hand, generally give very weak, non-specific signals when used to screen a library; thus, they are considered to be non-specific.

There are a range of formats for presenting recognition units used to screen libraries. Monovalent peptides, for example, synthesized peptides themselves, are non-specific. A peptide in the form of a bivalent fusion protein with alkaline phosphatase is very specific. The same peptide in the form of a fusion protein with the pIII protein of an M13 derived bacteriophage, expressed on the phage surface, has somewhat less, though still high, specificity. That same peptide when biotinylated in the form of a tetravalent streptavidin-alkaline phosphatase complex has generic specificity. Use of such a generically specific peptide permits the identification of a wide range of proteins from expression libraries or other sources of polypeptides, each protein containing an example of a particular PDZ domain.

Accordingly, the present invention provides a method of modulating the specificity of a peptide such that the peptide can be used as a recognition unit to screen a plurality of polypeptides, thus identifying polypeptides that have a PDZ domain. In a specific embodiment, specificity is generic so as to provide for the identification of polypeptides having a PDZ domain that varies in sequence from that of the known PDZ domain known to bind the recognition unit under conditions of high specificity. In a particular embodiment, the method comprises forming a tetravalent complex of the biotinylated peptide and streptavidin-alkaline phosphatase prior to use for screening an expression library.

According to the present invention, a recognition unit (preferably in the form of a multivalent recognition unit complex) is used to screen a plurality of expression products of gene sequences containing nucleic acid sequences that are present in native RNA or DNA (e.g., cDNA library, genomic library).

In a specific embodiment, the peptide recognition unit complex is in the form of a multivalent peptide complex comprising avidin or streptavidin (optionally conjugated to a label such as alkaline phosphatase or horseradish peroxidase) and biotinylated peptides. In a specific embodiment, recognition unit complexes are streptavidin complexed with 12 amino acid sequences having the Xaa-Ser/Thr-Xaa-Val-COOH motif at the C-terminal and a spacer sequence, e.g., Ser-Gly-Ser-Gly (SGSG) (SEQ ID NO:89) or Lys-Gly-Lys-Gly (SEQ ID NO:90) at their N-terminal, linked to biotin. In a preferred embodiment, the peptide sequence is one of those listed in FIGS. 5A and 5B, SEQ ID NOS:30-75 and 119.

In another specific embodiment, multivalent peptide PDZ domain recognition units may be in the form of multiple antigen peptides (MAP) (Tam, 1989, J. Imm. Meth. 124:53-61; Tam, 1988, Proc. Natl. Acad. Sci. USA 85:5409-5413). In this form, the peptide recognition unit is synthesized on a branching lysyl matrix using solid-phase peptide synthesis methods. Recognition units in the form of MAP may be prepared by methods known in the art (Tam, 1989, J. Imm. Meth. 124:53-61; Tam, 1988, Proc. Natl. Acad. Sci. USA 85:5409-5413), or, for example, by a stepwise solid-phase procedure on MAP resins (Applied Biosystems), utilizing methodology established by the manufacturer. MAP peptides may be synthesized comprising (recognition unit peptide)₂Lys₁, (recognition unit peptide)₄Lys₃, (recognition unit peptide)₆Lys₆ or more levels of branching.

The multivalent peptide recognition unit complexes may also be prepared by cross-linking the peptide to a carrier protein, e.g., bovine serum albumin (BSA), keyhole limpet hemocyanin (KLH) by use of known cross-linking reagents. Such cross-linked peptide recognition units may be detected by, example, an antibody to the carrier protein or detection of the enzymatic activity of the carrier protein. Other methods of routinely generating multiunit, multivalent forms of recognition unit(s) are known in the art and are encompassed by the invention.

The recognition units of the invention are used for screening polypeptides to identify PDZ domains as described in Sections 5.1.3 and 6.1. The recognition units also are used in defining the binding specificity of each PDZ domain via cross affinity mapping. The combination of a PDZ domain with a specific recognition unit allows an assay to be formatted that reflects the binding characteristics of the PDZ domain. The assay then allows for drug discovery assays wherein prospective drug candidates are added to the assay and their effect on the recognition unit-PDZ domain binding interaction is determined. In this way, compounds that inhibit or enhance the binding of PDZ domain containing proteins to their ligands can be identified.

### 5.1.3. Screening a Source of Polypeptides

After the recognition unit is chosen, the recognition unit or recognition unit complex is then contacted with a plurality of polypeptides. In a particular embodiment of the invention, the plurality of polypeptides is obtained from a polypeptide expression library. The polypeptide expression library may be obtained, in turn, from cDNA, fragmented genomic DNA, and the like. In a specific embodiment, the library that is screened is a cDNA library of total poly A RNA of an organism, generally, or of a particular cell or tissue type, developmental stage, or disease condition or stage. The expression library may utilize a number of expression vehicles known to those of ordinary skill, including but not limited to, recombinant bacteriophage, lambda phage, M13, a recombinant plasmid or cosmid, and the like.

The plurality of polypeptides or the DNA sequences encoding the same may be obtained from a variety of natural or unnatural sources, such as, for example, a procaryotic or a eucaryotic cell, which is either a wild type, recombinant, or mutant. In particular, the plurality of polypeptides may be endogenous to microorganisms, such as bacteria, yeast, or fungi, to a virus, to an animal (including mammals, invertebrates, reptiles, birds, and insects) or to a plant cell.

In addition, the plurality of polypeptides may be obtained from more specific sources, such as the surface coat of a virion particle, a particular cell lysate, a tissue extract, or the plurality of polypeptides may be restricted to those polypeptides that are expressed on the surface of a cell membrane.

Moreover, the plurality of polypeptides may be obtained from a biological fluid, particularly from humans, including but not limited to blood, plasma, serum, urine, feces, mucus, semen, vaginal fluid, amniotic fluid, or cerebrospinal fluid. The plurality of polypeptides may even be obtained from a fermentation broth or a conditioned medium, including all the polypeptide products secreted or produced by the cells previously in the broth or medium.

In a specific embodiment, the plurality of peptides are expressed by cDNA libraries made from mRNA isolated from human brain, heart, pituitary, spinal cord, colorectal carcinoma or prostate carcinoma tissue, as further described in Section 6.1.

The step of contacting the recognition unit with the plurality of polypeptides may be effected in a number of ways. For example, one may contemplate immobilizing the recognition unit on a solid support and bringing a solution of the plurality of polypeptides in contact with the immobilized recognition unit. Such a procedure would be akin to an affinity chromatographic process, with the affinity matrix being comprised of the immobilized recognition unit. The polypeptides having a selective affinity for the recognition unit can then be purified by affinity selection. The nature of the solid support, process for attachment of the recognition unit to the solid support, solvent, and conditions of the affinity isolation or selection procedure would depend on the type of recognition unit in use but would be largely conventional and well known to those of ordinary skill in the art. Moreover, the valency of the recognition unit in the recognition unit complex used to screen the polypeptides is believed to affect the specificity of the screening step, and thus the valency can be chosen as appropriate in view of the desired specificity (see Section 5.1.2).

Alternatively, one may also separate the plurality of polypeptides into substantially separate fractions comprising individual polypeptides. For instance, one can separate the plurality of polypeptides by gel electrophoresis, column chromatography, or like method known to those of ordinary skill for the separation of polypeptides. The individual polypeptides can also be produced by a transformed host cell in such a way as to be expressed on or about its outer surface. Individual isolates can then be “probed” by the recognition unit, optionally in the presence of an inducer should one be required for expression, to determine if any selective affinity interaction takes place between the recognition unit and the individual clone. Prior to contacting the recognition unit with each fraction comprising individual polypeptides, the polypeptides could first be transferred to a solid support for additional convenience. Such a solid support may simply be a piece of filter membrane, such as one made of nitrocellulose or nylon.

In this manner, positive clones could be identified from a collection of transformed host cells of an expression library, which harbor a DNA construct encoding a polypeptide having a selective affinity for the recognition unit. The polypeptide produced by the positive clone includes a PDZ domain or a functional equivalent thereof. Furthermore, the amino acid sequence of the polypeptide having a selective affinity for the recognition unit can be determined directly by conventional means or the coding sequence of the DNA encoding the polypeptide can frequently be determined more conveniently. The primary sequence can then be deduced from the corresponding DNA sequence and the PDZ domain identified.

If the amino acid sequence is to be determined from the polypeptide itself, one may use microsequencing techniques. The sequencing technique may include mass spectroscopy.

In certain situations, it may be desirable to wash away any unbound recognition unit from a mixture of the recognition unit and the plurality of polypeptides prior to attempting to determine or to detect the presence of a selective affinity interaction (i.e., the presence of a recognition unit that remains bound after the washing step). Such a wash step may be particularly desirable when the plurality of polypeptides is bound to a solid support.

In another embodiment, multiple recognition units are combined and contacted with a plurality of polypeptides according to the methods of the invention.

In specific embodiments, as many as fifty, twenty, ten, five, or two different recognition units are used simultaneously to screen a source of polypeptides. In particular, when the recognition units are biotinylated peptides and the source of polypeptides is a cDNA or genemic expression library, the steps of preconjugation of the biotinylated peptides to streptavidin-alkaline phosphatase as well as the steps involved in screening the cDNA expression library may be carried out in essentially the same manner as is performed when a single biotinylated peptide is used as a recognition unit. See Section 6.1 for details. The key difference when using more than one biotinylated peptide at a time is that the peptides are combined either before or at the step where they are placed in contact with the polypeptides from which selection occurs.

Those of ordinary skill in the art would appreciate that the clones testing positive for binding to a sample containing a plurality of recognition units, may routinely be tested against each of the biotinylated peptides contained in the sample to determine to which of the recognition units the clone binds.

The methods of the invention were applied to screen cDNA expression libraries with mixtures of peptide sequences having homology to known PDZ domains. In one experiment, peptides 2 (SEQ ID NO:58), 3 (SEQ ID NO:42), 5 (SEQ ID NO:30), and 7 (SEQ ID NO:32) were combined and used to screen a human brain cDNA library. In another embodiment, peptides 1-10 (SEQ ID NOS:49, 58, 42, 59, 30, 31, 32, 33, 51, 63, respectively) are combined to screen a plurality of polypeptides, specifically a human prostate carcinoma cell line LNCAP cDNA library. In a further experiment, peptides 27 (SEQ ID NO:40), 32 (SEQ ID NO:70), 33 (SEQ ID NO:71) and 34 (SEQ ID NO:72) were combined to screen the LNCAP cDNA library. In an additional experiment, a heart tissue cDNA library was screened with a mixture of peptides 1 (SEQ ID NO:49), 9 (SEQ ID NO:51), 31 (SEQ ID NO:62) and 34 (SEQ ID NO:72). In another experiment, a colorectal carcinoma cDNA library was screened with a mixture of peptides 25 (SEQ ID NO:38) and 26 (SEQ ID NO:39). Additionally, a pituitary cDNA library was screened with a mixture of peptides 15 (SEQ ID NO:56), 16 (SEQ ID NO:35), 17 (SEQ ID NO:66), 18 (SEQ ID NO:67), 6 (SEQ ID NO:31), 7 (SEQ ID NO:32), 8 (SEQ ID NO:33), and 9 (SEQ ID NO:51). In a further experiment, a spinal cord cDNA library was screened with peptides 34 (SEQ ID NO:72), 41 (SEQ ID NO:119), N1 (SEQ ID NO:73) and N3 (SEQ ID NO:74). The recognition unit responsible for identifying a PDZ containing clone according to the embodiments of the invention is easily identified in subsequent cross affinity mapping experimentation.

As shown in Table 8, the screening of the human brain cDNA library with a mixture of peptide recognition units which included peptide 3; NMDA glutamate receptor (SEQ ID NO:42)), resulted in the isolation of PSD-95, a known PDZ domain containing protein. It is known that the second PDZ domain of PSD-95 binds the peptide sequence of peptide 3. Interestingly, screening of the LNCAP cDNA library using mixtures of peptide recognition units that include peptide 3, led to identification of the novel proteins PDZP1 and PDZP2.

As can be anticipated, the degree of selective affinities observed varies widely, generally falling in the range of about 1 nm to about 1 mM. In preferred embodiments of the present invention, the selective affinity falls on the order of about 10 nM to about 100 μM, more preferably on the order of about 100 nM to about 10 μM, and most preferably on the order of about 100 nM to about 1 μM.

### 5.2. Kits

The present invention is also directed to an assay kit which can be useful in the screening of drug candidates. In a particular embodiment of the present invention, an assay kit is contemplated which comprises in one or more containers (a) a polypeptide containing a PDZ domain; and (b) a recognition unit having a selective affinity for the domain of the polypeptide. The kit optionally further comprises a detection means for determining the presence of a polypeptide-recognition unit interaction or the absence thereof.

In a specific embodiment, either the polypeptide containing the PDZ domain or the recognition unit is labeled. A wide range of labels can be used to advantage in the present invention, including but not limited to conjugating the recognition unit to biotin by conventional means. Alternatively, the label may comprise, for example, a fluorogen, an enzyme, an epitope, a chromogen, or a radionuclide. Preferably, the biotin is conjugated by covalent attachment to either the polypeptide or the recognition unit. The polypeptide or, preferably, the recognition unit is immobilized on a solid support. The detection means employed to detect the label will depend on the nature of the label and can be any known in the art, e.g., film to detect a radionuclide; an enzyme substrate that gives rise to a detectable signal to detect the presence of an enzyme; antibody to detect the presence of an epitope, etc.

A further embodiment of the assay kit of the present invention includes the use of a plurality of polypeptides, each polypeptide containing a PDZ domain. The assay kit further comprises at least one recognition unit having a selective affinity for each of the plurality of polypeptides and a detection means for determining the presence of a polypeptide-recognition unit interaction or the absence thereof.

In a further embodiment, a kit is provided that comprises, in one or more containers, a first molecule comprising a PDZ domain and a second molecule that binds to the PDZ domain, i.e., a recognition unit, where the PDZ domain is a novel PDZ domain identified by the methods of the present invention.

In the above assay kit, the polypeptide may comprise an amino acid sequence selected from the group consisting of SEQ ID NOS:10-27 and 111-116. The polypeptide also may comprise an amino acid sequence selected from the group consisting of SEQ ID NOS:76, 78, 80, 99, 101, and 103.

In other embodiments of the above-described assay kit, the recognition unit may be a peptide selected from the group consisting of SEQ ID NOS:30-74 and 119. The recognition unit may be labeled with e.g., an enzyme, an epitope, a chromogen, or biotin or other electrochemical means.

In a preferred embodiment, the recognition unit is a biotinylated peptide complexed with avidin or streptavidin. Even more preferred is a recognition unit complex comprising biotinylated peptides complexed with streptavidin-alkaline phosphatase. Alkaline phosphatase can then be detected using appropriate substrates, or alternatively, using the TSA-Tyramide signal amplification system (Dupont NEL-700).

The present invention also provides an assay kit comprising in one or more containers:
(a) a plurality of purified different polypeptides, each polypeptide in a separate container and each polypeptide containing a PDZ domain; and
(b) at least one peptide having a selective affinity for the PDZ domain in each of said plurality of polypeptides, which optionally, if present as more than one peptide, each peptide can also be in a separate container.

The present invention also provides a kit comprising a plurality of purified polypeptides comprising a PDZ domain, each polypeptide separated from the other, and each polypeptide having a PDZ domain of a different sequence, but capable of displaying the same binding specificity (binding to the same molecule under appropriate conditions). In specific embodiments, the polypeptides are separated on a fixed substrate such as, for example, a plate or gel. In another specific embodiment, the polypeptides are in separate containers or wells.

In the above-described kits, the polypeptides may have an amino acid sequence selected from the group consisting of: SEQ ID NOS:10-27 and 111-116. The polypeptides also may have an amino acid sequence selected from the group consisting of SEQ ID NOS:76, 78, 80, 99, 101, and 103.

The components of the kits are preferably purified.

The kits of the present invention may be used in the methods for identifying new drug candidates and determining the specificities thereof that are described in Section 5.4.

### 5.3. Assays for the Discovery of Potential Drug Candidates and Determining the Specificity Thereof

A common problem in the development of new drugs is that of identifying a single, or a small number, of compounds that possess a desirable characteristic from among a background of many compounds that lack that desired characteristic. This problem arises both in the testing of compounds that are natural products from plant, animal, or microbial sources and in the testing of man-made compounds. Typically, hundreds, or even thousands, of compounds are randomly screened by the use of in vitro assays such as those that monitor the compound's effect on some enzymatic activity, its ability to bind to a reference substance such as a receptor or other protein, or its ability to disrupt the binding between a receptor and its ligand.

The compounds which pass this original screening test are known as “lead” compounds. These lead compounds are then put through further testing, including, eventually, in vivo testing in animals and humans, from which the promise shown by the lead compounds in the original in vitro tests is either confirmed or refuted. See Remington's Pharmaceutical Sciences, 1990, A. R. Gennaro, ed., Chapter 8, pages 60-62, Mack Publishing Co., Easton, Pa.; Ecker and Crooke, 1995, Bio/Technology 13:351-360.

There is a continual need for new compounds to be tested in the in vitro assays that make up the first testing step described above. There is also a continual need for new assays by which the pharmacological activities of these compounds may be tested. It is an object of the present invention to provide such new assays to determine whether a candidate compound is capable of affecting the binding between a polypeptide containing a PDZ domain and a recognition unit that binds to that PDZ domain. In particular, it is an object of the present invention to provide polypeptides, particularly novel ones, containing PDZ domains and their corresponding recognition units for use in the above-described assays. The use of these polypeptides greatly expands the number of assays that may be used to screen potential drug candidates for useful pharmacological activities (as well as to identify potential drug candidates that display adverse or undesirable pharmacological activities).

The present invention also provides methods for identifying potential drug candidates (and lead compounds) and determining the specificities thereof. For example, knowing that a polypeptide containing a PDZ domain and a recognition unit, e.g., a binding peptide, exhibit a selective affinity for each other, one may proceed with identifying a drug that can exert an effect on the polypeptide-recognition unit interaction, e.g., either as an agonist or as an antagonist (inhibitor) of the interaction. With this assay, then, one can screen a collection of candidate “drugs” for the one exhibiting the most desired characteristic, e.g., the most efficacious in disrupting the interaction or in competing with the recognition unit for binding to the polypeptide.

Alternatively, one may utilize the different selectivities that a particular recognition unit may exhibit for different polypeptides bearing the same, similar, or functionally equivalent PDZ domains. Thus, one may tailor the screen to identify drug candidates that exhibit more selective activities directed to specific polypeptide-recognition unit interactions, among the “panel” of possibilities. Thus, for example, a drug candidate may be screened to identify the presence or absence of an effect on particular binding interactions, potentially leading to undesirable side effects.

In one embodiment, the effect of the drug candidate upon multiple, different interacting polypeptide-recognition unit pairs is determined in which at least some of said polypeptides have a PDZ domain that differs in sequence, but is capable of displaying the similar binding specificity to a recognition unit as the PDZ domain in another of said polypeptides.

In another embodiment, at least one of said polypeptides or recognition units contains a consensus PDZ domain and consensus recognition unit, respectively.

In another embodiment, the drug candidate is an inhibitor of the polypeptide-recognition unit interaction that is identified by detecting a decrease in the binding of polypeptide to recognition unit in the presence of such inhibitor.

In another embodiment, said polypeptide is a polypeptide containing a PDZ domain identified according to the methods of the invention (see e.g., Section 5.1).

In a specific embodiment, the polypeptide is a novel polypeptide PDZP1, PDZP2, PDZP3, PDZP4, PDZP5 and/or specific PDZ domains from these polypeptides or from KIAA-147 identified by the methods of the present invention.

One of ordinary skill in the art will recognize that it will not always be necessary to utilize the entire novel or known polypeptide which contains one or more PDZ domains in the assays described herein. Often, a portion of the polypeptide that contains the PDZ domain will be sufficient, e.g., a glutathione S-transferase (GST)-PDZ domain fusion protein. See FIGS. 3A and 3B for a depiction of the portions of the exemplary novel polypeptides that contain PDZ domains.

A typical assay of the present invention consists of at least the following components: (1) a molecule (e.g., protein or polypeptide) comprising a PDZ domain; (2) a recognition unit that selectively binds to the PDZ domain; (3) a candidate compound, suspected of having the capacity to affect the binding between the protein containing the PDZ domain and the recognition unit. The assay components may further comprise (4) a means of detecting the binding of the protein comprising the PDZ domain and the recognition unit. Such means can be, for example, a detectable label affixed to the protein comprising the PDZ domain, the recognition unit, or the candidate compound. In a specific embodiment, the protein comprising the PDZ domain is a novel protein discovered by the methods of the present invention.

In another specific embodiment, the invention provides a method of identifying a compound that affects the binding of a molecule comprising a PDZ domain and a recognition unit that selectively binds to the PDZ domain comprising:
(a) contacting the molecule comprising the PDZ domain and the recognition unit under conditions conducive to binding and measuring the amount of binding between the molecule and the recognition unit;
(b) contacting the molecule comprising the PDZ domain and the recognition unit as in step (a), but in the presence of a candidate compound; and
(c) comparing the amount of binding in step (a) with the amount of binding in step (b), where a difference indicates that the candidate compound is a compound that affects the binding of the molecule comprising a PDZ domain and the recognition unit. In a specific embodiment, the compound is not a peptide.

In another specific embodiment, the invention provides a method of identifying a compound that affects the binding of a molecule comprising a PDZ domain and a recognition unit that selectively binds to the PDZ domain comprising:
(a) contacting the molecule comprising the PDZ domain and the recognition unit under conditions conducive to binding and measuring the amount of binding between the molecule and the recognition unit in which the PDZ domain has an amino acid comprising one of the novel PDZ domains depicted in FIGS. 3A and 3B (SEQ ID NOS:10-27 and 111-116);
(b) contacting the molecule comprising the PDZ domain and the recognition unit under as in step (a), but in the presence of a candidate protein; and
(c) comparing the amount of binding in step (a) with the amount of binding in step (b), where a difference indicates that the candidate compound is a compound that affects the binding of the molecule comprising a PDZ domain and the recognition unit.

In another specific embodiment, the invention provides a method of identifying a compound that affects the binding of a molecule comprising a PDZ domain and a recognition unit that selectively binds to the PDZ domain comprising:
(a) contacting the molecule comprising the PDZ domain and the recognition unit under conditions conducive to binding and measuring the amount of binding between the molecule and the recognition unit in which the recognition unit comprises an amino acid sequence selected from SEQ ID NOS:30-75 and 119; and
(b) contacting the molecule comprising the PDZ domain and the recognition unit under as in step (a), but in the presence of a candidate compound; and
(c) comparing the amount of binding in step (a) with the amount of binding in step (b) where a difference indicates that the candidate compound is a compound that affects the binding of the molecule comprising a PDZ domain and the recognition unit.

It is possible to determine whether the candidate compound affects the binding and thus is a useful lead compound for the modulation of the activity of polypeptides containing the PDZ domain. The effect of the candidate compound may be to either increase or decrease the binding.

One version of an assay suitable for use in the present invention comprises binding the polypeptide containing a PDZ domain to a solid support such as the wells of a microtiter plate. The wells contain a suitable buffer and other substances to ensure that conditions in the wells permit the binding of the polypeptide containing a PDZ domain to its recognition unit. The recognition unit and a candidate compound are then added to the wells. The recognition unit is preferably labeled, e.g., it might be biotinylated or labeled with a radioactive moiety, or it might be linked to an enzyme, e.g., alkaline phosphatase. After a suitable period of incubation, the wells are washed to remove any unbound recognition unit and compound. If the candidate compound does not interfere with the binding of the polypeptide containing a PDZ domain to the labeled recognition unit, the labeled recognition unit will bind to the polypeptide containing a PDZ domain in the well. This binding can then be detected. If the candidate compound interferes with the binding of the polypeptide containing a PDZ domain and the labeled recognition unit, label will not be present in the wells, or will be present to a lesser degree than is the case when compared to control wells that contain the polypeptide containing a PDZ domain and the labeled recognition unit, but to which no candidate compound is added. Of course, it is possible that the presence of the candidate compound will increase the binding between the polypeptide containing a PDZ domain and the labeled recognition unit. Alternatively, the recognition unit can be affixed to a solid substrate during the assay.

In a specific embodiment, the methods of the invention are utilized to identify potential drug candidates (and lead compounds) for the treating and preventing of brain injury resulting from stroke. In normal neurological synaptic physiology, neurons release the neurotransmitter glutamate from the presynaptic membrane. Glutamate binds and activates N-methy-D aspartate receptor (NMDAR) which as a calcium channel, then allows an influx of calcium into the postsynaptic cell. Calcium inside the cell binds to calmodulin. Calcium binding to calmodulin activates Nitric oxide synthase (NOS) which converts L-Arginine to release NO⁺, an endogenous signaling molecule.

When cerebral ischemia occurs in animal models of Parkinson's disease, blood flow to the brain decreases, decreasing oxygen flow, which in-turn, causes an increases in glutamate release from the presynaptic neuron. This ultimately leads to an increase in nitric oxide which reaches toxic levels leading to brain cell death. Stricker, 1997, Nat. Biotechnol. 15:336-34.

Drugs to potentially protect brain cells in the event of stroke have been developed that either target NMDAR to prevent calcium influx or that inhibit NOS to block the increase in nitric oxide. Compounds studied to date are not particularly effective due to numerous reasons which include lack of brain tissue and target molecule specific activity.

A protein inhibitor of NOS has been described. Jaffrey et al, 1996, Science 274:774-777. More particular regulation of NOS is seen with molecular targeting of NOS to specific intracellular membrane domains. Aski et al., 1993, Brain Res. 620:97-113. The subcellular localization is mediated by the N-terminus of NOS, which contains a PDZ domain. Brenman et al., 1995, Cell 82:743-752. This N-terminal domain of NOS also interacts with the PDZ domain of α1-Syntrophin, PSD95-PDZ2, and PSD93-PDZ2. Brenman et al., 1996, Cell 84:757-767.

In an embodiment of the current invention, the PDZ domains of PSD-95 and NOS are targeted as sights for drug intervention aiming to uncouple calmodulin and NOS from NMDAR to prevent the cascade of events leading to NO⁺accumulation in brain cells in stroke. According to one embodiment of the invention, potential drug compounds are screened for ability to inhibit specifically the binding of the PDZ1 domain of PSD-95 (SEQ ID NO:109) to NMDAR (SEQ ID NOS:42-48); PDZ3 domain of PSD-95 (SEQ ID NO:9) to NMDAR (SEQ ID NOS:42-48); and PDZ2 of PSD95 (SEQ ID NO:110) to the PDZ domain of NOS (SEQ ID NO: ______ (see FIG. 5)). Generally, the method for screening for a potential drug candidate comprises:
(a) allowing at least one polypeptide comprising a PDZ domain to come into contact with at least one recognition unit having a selective affinity for said PDZ domain in said polypeptide, in the presence of an amount of a potential drug candidate, such that said polypeptide and said recognition unit are capable of interacting when brought into contact with one another in the absence of said drug candidate; and
(b) determining the effect, if any, of the presence of the amount of said drug candidate on the interaction of said polypeptide with said recognition unit.

In a specific embodiment, the effect of the drug candidate upon multiple, different interacting polypeptide-recognition unit pairs is determined. In a further embodiment, at least some of the polypeptides have a PDZ domain that differs in sequence but is capable of displaying the same binding specificity as the PDZ domain in another of the polypeptides. In another specific embodiment, at least one of the polypeptides or recognition units contain a consensus PDZ domain and consensus recognition unit, respectively. In another specific embodiment, the polypeptide used in step (a) of the method of screening for a potential drug candidate is identified by a method comprising:
(i) contacting a multivalent recognition unit complex having selective binding affinity for a PDZ domain with a plurality of polypeptides; and
(ii) identifying a polypeptide having a selective binding affinity for said recognition unit complex.

In an additional specific embodiment, the drug candidate utilized according to the method of screening of the invention is an inhibitor of the polypeptide-recognition unit interaction that is identified by detecting a decrease in the binding of polypeptide to recognition unit in the presence of such inhibitor.

### 5.4. Use of Polypeptides Containing PDZ Domains to Discover Polypeptides Involved in Pharmacological Activities

Using the methods of the present invention, it is possible to identify and isolate large numbers of polypeptides containing PDZ domains. Using these polypeptides, one can construct a matrix relating the polypeptides to an array of candidate drug compounds. For example, FIGS. 5A and 5B shows such a matrix or cross affinity map.

Assays that generate the data, e.g., that of FIGS. 5A and 5B, are conducted in the presence of various compounds that potentially effect the binding of the domains to respective recognition units. From this data compounds with various pharmacologic activities are identified.

Compound A generally effects a wide variety of PDZ domain-ligand interactions, Table 5. Compound B is specific for interactions characterized by ligands having the C terminal sequence ESKV, Table 6. Compound C is specific for PDZP2 interactions, Table 7.

TABLE 5
PDZ domain-recognition unit interactions in the presence of Compound A

| | PDZ Domain/GST Fusion Proteins | | | | | | |
|---|---|---|---|---|---|---|---|
| Recognition Unit | PDZP2.1 | PDZ2.2 | PDZ2.3 | PDZ2.4 | PDZ3.1 | PDZ3.2 | PSD-95-1 |
| SEQ ID NO. 30 | + | + | − | − | + | − | − |
| SEQ ID NO. 31 | + | + | − | + | + | − | − |
| SEQ ID NO. 32 | − | − | − | + | + | − | − |
| SEQ ID NO. 33 | − | − | + | + | + | − | − |
| SEQ ID NO. 34 | − | − | − | − | + | − | − |
| SEQ ID NO. 35 | + | + | − | − | + | − | − |
| SEQ ID NO. 36 | − | − | − | − | + | − | − |
| SEQ ID NO. 37 | + | + | + | + | − | − | + |
| SEQ ID NO. 38 | + | + | + | + | − | − | + |
| SEQ ID NO. 39 | + | + | + | + | + | + | + |
| SEQ ID NO. 40 | + | + | + | + | + | + | + |
| SEQ ID NO. 41 | − | − | + | − | + | − | + |
| SEQ ID NO. 42 | + | + | + | + | + | − | − |
| SEQ ID NO. 43 | − | − | − | + | − | − | − |
| SEQ ID NO. 44 | − | − | − | − | − | − | − |
| SEQ ID NO. 45 | − | − | − | − | − | − | − |
| SEQ ID NO. 46 | − | − | − | − | − | − | − |
| SEQ ID NO. 47 | − | − | − | − | − | − | − |
| SEQ ID NO. 48 | − | − | − | − | − | − | − |
| SEQ ID NO. 49 | + | + | + | − | + | − | + |
| SEQ ID NO. 50 | − | − | − | − | − | − | + |
| SEQ ID NO. 51 | + | + | ++ | + | + | + | + |
| SEQ ID NO. 52 | − | − | − | − | − | − | − |

TABLE 6
PDZ domain-recognition unit interactions in the presence of Compound B

| | PDZ Domain/GST Fusion Proteins | | | | | | |
|---|---|---|---|---|---|---|---|
| Recognition Unit | PDZP2.1 | PDZ2.2 | PDZ2.3 | PDZ2.4 | PDZ3.1 | PDZ3.2 | PSD-95-1 |
| SEQ ID NO. 30 | − | − | − | − | − | − | − |
| SEQ ID NO. 31 | ++++ | ++++ | ++ | ++++ | ++++ | ++ | ++ |
| SEQ ID NO. 32 | − | − | − | ++++ | ++ | − | − |
| SEQ ID NO. 33 | + | + | ++++ | +++ | ++++ | − | ++ |
| SEQ ID NO. 34 | + | − | − | − | ++++ | − | − |
| SEQ ID NO. 35 | − | − | − | − | − | − | − |
| SEQ ID NO. 36 | − | − | − | − | ++++ | + | − |
| SEQ ID NO. 37 | ++++ | ++++ | ++++ | +++ | − | − | +++ |
| SEQ ID NO. 38 | ++++ | ++++ | ++++ | +++ | − | − | +++ |
| SEQ ID NO. 39 | ++++ | ++++ | ++++ | ++++ | ++++ | ++++ | +++ |
| SEQ ID NO. 40 | ++++ | ++++ | ++++ | ++++ | ++++ | +++ | ++++ |
| SEQ ID NO. 41 | ++ | ++ | ++++ | − | ++++ | − | +++ |
| SEQ ID NO. 42 | ++++ | +++ | ++++ | ++++ | ++++ | − | +++ |
| SEQ ID NO. 43 | − | − | − | +++ | − | − | − |
| SEQ ID NO. 44 | − | − | − | − | − | − | − |
| SEQ ID NO. 45 | − | − | − | − | − | − | − |
| SEQ ID NO. 46 | − | − | − | − | − | − | − |
| SEQ ID NO. 47 | − | − | − | − | − | − | − |
| SEQ ID NO. 48 | − | − | − | − | − | − | − |
| SEQ ID NO. 49 | ++++ | ++++ | ++++ | − | ++++ | − | ++++ |
| SEQ ID NO. 50 | − | − | − | − | − | − | +++ |
| SEQ ID NO. 51 | ++++ | ++++ | ++++ | ++++ | ++++ | ++++ | +++ |
| SEQ ID NO. 52 | − | − | − | − | − | − | − |

TABLE 7
PDZ domain-recognition unit interactions in the presence of Compound C

| | PDZ Domain/GST Fusion Proteins | | | | | | |
|---|---|---|---|---|---|---|---|
| Recognition Unit | PDZP2.1 | PDZ2.2 | PDZ2.3 | PDZ2.4 | PDZ3.1 | PDZ3.2 | PSD-95-1 |
| SEQ ID NO. 30 | − | − | − | − | ++++ | − | − |
| SEQ ID NO. 31 | + | + | − | + | ++++ | ++ | ++ |
| SEQ ID NO. 32 | − | − | − | + | ++ | − | − |
| SEQ ID NO. 33 | + | + | + | + | ++++ | − | ++ |
| SEQ ID NO. 34 | + | − | − | − | ++++ | − | − |
| SEQ ID NO. 35 | + | + | − | − | ++++ | ++ | ++ |
| SEQ ID NO. 36 | − | − | − | − | ++++ | + | − |
| SEQ ID NO. 37 | + | + | + | + | − | − | +++ |
| SEQ ID NO. 38 | + | + | + | + | − | − | +++ |
| SEQ ID NO. 39 | + | + | + | + | ++++ | ++++ | +++ |
| SEQ ID NO. 40 | + | + | + | + | ++++ | +++ | ++++ |
| SEQ ID NO. 41 | + | − | + | − | ++++ | − | +++ |
| SEQ ID NO. 42 | + | + | + | + | ++++ | − | +++ |
| SEQ ID NO. 43 | − | − | − | + | − | − | − |
| SEQ ID NO. 44 | − | − | − | − | − | − | − |
| SEQ ID NO. 45 | − | − | − | − | − | − | − |
| SEQ ID NO. 46 | − | − | − | − | − | − | − |
| SEQ ID NO. 47 | − | − | − | − | − | − | − |
| SEQ ID NO. 48 | − | − | − | − | − | − | − |
| SEQ ID NO. 49 | + | + | + | − | ++++ | − | ++++ |
| SEQ ID NO. 50 | − | − | − | − | − | − | +++ |
| SEQ ID NO. 51 | + | + | + | + | ++++ | ++++ | +++ |
| SEQ ID NO. 52 | − | − | − | − | − | − | − |

Data is collected for various compounds as in the above cross affinity maps. The data is correlated with known or deduced physiological activities of either the PDZ domain containing polypeptide, the PDZ domain ligand or the tested compounds. For example, if ESKV specific PDZ domains are associated with a specific physiological activity that it is desired to effect, then compound B is a good candidate as a drug lead. In another case, PDZP2.2 might be associated with a particular pharmacological activity. Compound C could be a good drug lead—however, the compound's effect on PDZP2.1, PDZP2.3 and PDZP2.4 might result in undesired side effects and should be evaluated further. Alternatively, a compound specific for PDZP2.2 could be identified.

Such data is used to determine whether novel polypeptides or other candidate drug compounds display or are at risk of displaying desirable or undesirable physiological or pharmacological activities.

As the maps are generated and pharmacological effects observed, the maps will allow strategic assessment of the specificity necessary to obtain the desired pharmacological effect.

Accordingly, the methods of the present invention providing for assays utilizing the polypeptides comprising PDZ domains of the present invention can be used to determine the participation of those polypeptides in pharmacological activities.

### 5.5. Isolation and Expression of Nucleic Acid Encoding Polypeptides Comprising a PDZ Domain

In particular aspects, the invention provides amino acid sequences of polypeptides comprising PDZ domains, preferably human polypeptides, and fragments and derivatives thereof which comprise an antigenic determinant (i.e., can be recognized by an antibody) or which are functionally active, as well as nucleic acid sequences encoding the foregoing. “Functionally active” material as used herein refers to that material displaying one or more functional activities, such as, for example, a biological activity, antigenicity, immunogenicity, or comprising a PDZ domain that is capable of specific binding to a recognition unit. In specific embodiments, the invention provides fragments of polypeptides comprising a PDZ domain, or portion thereof, consisting of at least 40, 50, 60, 70, 80, 90, 100, 120, 150 or 200 amino acids. Nucleic acids encoding the foregoing are provided.

In other specific embodiments, the invention provides nucleotide sequences and subsequences encoding polypeptides comprising a PDZ domain, or portion thereof, preferably human polypeptides, consisting of at least 13 nucleotides, at least 50 nucleotides, at least 100 nucleotides, at least 150 nucleotides, at least 200 nucleotides, at least 250 nucleotides at least 300 nucleotides, at least 350 nucleotides or at least 450 nucleotides. Nucleic acids encoding fragments of the polypeptides comprising a PDZ domain are provided, as well as nucleic acids complementary to and capable of hybridizing to such nucleic acids. In one embodiment, such a complementary sequence may be complementary to a cDNA sequence encoding a polypeptide comprising a PDZ domain, or portion thereof, of at least 13, 25, 50, 100, 150, 200, 250, or at least 300 nucleotides. In a preferred aspect, the invention utilizes cDNA sequences encoding human polypeptides comprising a PDZ domain or a portion thereof.

Any eukaryotic cell can potentially serve as the nucleic acid source for the molecular cloning of polypeptides comprising a PDZ domain. The DNA may be obtained by standard procedures known in the art (e.g., a DNA “library”) by cDNA cloning, or by the cloning of genomic DNA, or fragments thereof, purified from the desired cell (see, for example Sambrook et al., 1989, Molecular Cloning, A Laboratory Manual, Cold Spring Harbor Laboratory, 2d. Ed., Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences. Whatever the source, the gene encoding a polypeptide comprising a PDZ domain should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once a gene encoding a particular polypeptide comprising a PDZ domain has been isolated from a first species, it is a routine matter to isolate the corresponding gene from another species. Identification of the specific DNA fragment from another species containing the desired gene may be accomplished in a number of ways. For example, if an amount of a portion of a gene or its specific RNA from the first species, or a fragment thereof e.g., the PDZ domain, is available and can be purified and labeled, the generated DNA fragments from another species may be screened by nucleic acid hybridization to the labeled probe (Benton, W. and Davis, R., 1977, Science 196:180; Grunstein, M. And Hogness, D., 1975, Proc. Natl. Acad. Sci. U.S.A. 72:3961). Those DNA fragments with substantial homology to the probe will hybridize. In a preferred embodiment, PCR using primers that hybridize to a known sequence of a gene of one species can be used to amplify the homolog of such gene in a different species. The amplified fragment can then be isolated and inserted into an expression or cloning vector. It is also possible to identify the appropriate fragment by restriction enzyme digestion(s) and comparison of fragment sizes with those expected according to a known restriction map if such is available. Further selection can be carried out on the basis of the properties of the gene. Alternatively, the presence of the gene may be detected by assays based on the physical, chemical, or immunological properties of its expressed product. For example, cDNA clones, or DNA clones which hybrid-select the proper mRNAs, can be selected which produce a protein that, for example, has similar or identical electrophoretic migration, isolectric focusing behavior, proteolytic digestion maps, in vitro aggregation activity (“adhesiveness”) or antigenic properties as known for the particular polypeptide comprising a PDZ domain from the first species. If an antibody to that particular polypeptide is available, the corresponding polypeptide from another species may be identified by binding of labeled antibody to the putative polypeptide synthesizing clones in an ELISA (enzyme-linked immunosorbent assay)-type procedure.

Genes encoding polypeptides comprising a PDZ domain can also be identified by mRNA selection by nucleic acid hybridization followed by in vitro translation. In this procedure, fragments are used to isolate complementary mRNAs by hybridization. Such DNA fragments may represent available, purified DNA of genes encoding polypeptides comprising a PDZ domain of a first species. Immunoprecipitation analysis or functional assays (e.g., ability to bind to a recognition unit) of the in vitro translation products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against polypeptides comprising a PDZ domain. A radiolabelled cDNA of a gene encoding a polypeptide comprising a PDZ domain can be synthesized using the selected mRNA (from the adsorbed polysomes) as a template. The radiolabelled mRNA or cDNA may then be used as a probe to identify the DNA fragments that represent the gene encoding the polypeptide comprising a PDZ domain of another species from among other genomic DNA fragments. In various embodiments, the nucleic acid used as a probe is hybridizable to a homolog from another species under conditions of low, moderate, or high stringency. By way of example and not limitation, procedures using such conditions of low stringency are as follows (see also Shilo and Weinberg, 1981, Proc. Natl. Acad. Sci. USA 78:6789-6792): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

By way of example and not limitation, procedures using conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

The identified and isolated gene encoding a polypeptide comprising a PDZ domain can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and gene may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shot gun” approach. Enrichment for the desired gene, for example, by size fractionization, can be done before insertion into the cloning vector.

In specific embodiments, transformation of host cells with recombinant DNA molecules that incorporate the isolated gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleic acid coding for a polypeptide comprising a PDZ domain of the invention can be inserted into an appropriate expression vector, i.e., a vector which contains the necessary elements for the transcription and translation of the inserted protein-coding sequence. The necessary transcriptional and translational signals can also be supplied by the native gene encoding the polypeptide and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Any of the methods previously described for the insertion of DNA fragments into a vector may be used to construct expression vectors containing a chimeric gene consisting of appropriate transcriptional/translational control signals operably linked to the protein coding sequences. These methods may include in vitro recombinant DNA and synthetic techniques and in vivo recombinants (genetic recombination). Expression of nucleic acid sequence encoding a protein or peptide fragment may be regulated by a second nucleic acid sequence so that the protein or peptide is expressed in a host transformed with the recombinant DNA molecule. For example, expression of a protein may be controlled by any promoter/enhancer element known in the art. Promoters which may be used to control gene expression include, but are not limited to, the SV40 early promoter region (Benoist and Chambon, 1981, Nature 290:304-310), the romoter contained in the 31 long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff et al., 1978, Proc. Natl. Acad. Sci. U.S.A. 75:3727-3731), or the tac promoter (DeBoer et al., 1983, Proc. Natl. Acad. Sci. U.S.A. 80:21-25); see also “Useful proteins from recombinant bacteria” in Scientific American, 1980, 242:74-94; plant expression vectors comprising the nopaline synthetase promoter region (Herrera-Estrella et al., Nature 303:209-213) or the cauliflower mosaic virus 35S RNA promoter (Gardner et al., 981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315, 115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Expression vectors containing inserts of genes encoding polypeptides comprising a PDZ domain can be identified by three general approaches: (a) nucleic acid hybridization, (b) presence or absence of “marker” gene functions, and (c) expression of inserted sequences. In the first approach, the presence of a foreign gene inserted in an expression vector can be detected by nucleic acid hybridization using probes comprising sequences that are homologous to the inserted gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign genes in the vector. For example, if the gene encoding a polypeptide comprising a PDZ domain is inserted within the marker gene sequence of the vector, recombinants containing the gene can be identified by the absence of the marker gene function. In the third approach, recombinant expression vectors can be identified by assaying the foreign gene product expressed by the recombinant. Such assays can be based, for example, on the physical or functional properties of the gene product in vitro assay systems (e.g., ability to bind to recognition units).

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Expression from certain promoters can be elevated in the presence of certain inducers; thus, expression of the protein may be controlled. Furthermore, different host cells have characteristic and specific mechanisms for the translational and post-translational processing and modification (e.g., glycosylation, cleavage) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions such as proteolytic cleavages to different extents.

In other specific embodiments, polypeptides comprising a PDZ domain, or fragments, analogs, or derivatives thereof may be expressed as a fusion, or chimeric protein product (comprising the polypeptide, fragment, analog, or derivative joined via a peptide bond to a heterologous protein sequence (of a different protein)). Such a chimeric product can be made by ligating the appropriate nucleic acid sequences encoding the desired amino acid sequences to each other by methods known in the art, in the proper reading frame, and expressing the chimeric product by methods commonly known in the art. Alternatively, such a chimeric product may be made by protein synthetic techniques (e.g., by use of a peptide synthesizer).

### 5.5.1. Identification and Purification of the Expressed Gene Products

Once a recombinant which expresses the gene sequence encoding a polypeptide comprising a PDZ domain is identified, the gene product may be analyzed. This can be achieved by assays based on the physical or functional properties of the product, including radioactive labelling of the product followed by analysis by gel electrophoresis.

Once the polypeptide comprising a PDZ domain is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay, including, but not limited to, binding to a recognition unit.

### 5.6. Derivatives and Analogs of Polypeptides Comprising a PDZ Domain

The invention further provides derivatives (including but not limited to fragments) and analogs of polypeptides comprising a PDZ domain. In a specific embodiment, the derivative or analog is functionally active, i.e., capable of exhibiting one or more functional activities associated with a full-length, wild-type polypeptide, e.g., binding to a recognition unit. As one example, such derivatives or analogs may have the antigenicity of the full-length polypeptide.

In particular, derivatives can be made by altering gene sequences encoding polypeptides comprising a PDZ domain by substitutions, additions, or deletions that provide for functionally equivalent molecules. Due to the degeneracy of nucleotide coding sequences, other DNA sequences which encode substantially the same amino acid sequence as a gene encoding a polypeptide comprising a PDZ domain may be used in the practice of the present invention. These include but are not limited to nucleotide sequences comprising all or portions of such genes which are altered by the substitution of different codons that encode a functionally equivalent amino acid residue within the sequence, thus producing a silent change. Likewise, the derivatives of the invention include, but are not limited to, those containing, as a primary amino acid sequence, all or part of the amino acid sequence of a polypeptide comprising a PDZ domain including altered sequences in which functionally equivalent amino acid residues are substituted for residues within the sequence, resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Derivatives or analogs of genes encoding polypeptides comprising a PDZ domain include but are not limited to those polypeptides which are substantially homologous to the genes or fragments thereof, or whose encoding nucleic acid is capable of hybridizing to a nucleic acid sequence of the genes.

The derivatives and analogs of the invention can be produced by various methods known in the art. The manipulations which result in their production can occur at the gene or protein level. For example, the cloned gene sequence can be modified by any of numerous strategies known in the art (Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. PCR primers can be constructed so as to introduce desired sequence changes during PCR amplification of a nucleic acid encoding the desired polypeptide. In the production of the gene encoding a derivative or analog, care should be taken to ensure that the modified gene remains within the same translational reading frame, uninterrupted by translational stop signals, in the gene region where the desired activity is encoded.

Additionally, the sequence of the genes encoding polypeptides comprising a PDZ domain can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson et al., 1978, J. Biol. Chem 253:6551), use of TAB® linkers (Pharmacia, Piscataway, N.J.), etc.

Manipulations of the sequence may also be made at the protein level. Included within the scope of the invention are protein fragments or other derivatives or analogs which are differentially modified during or after translation, for example, by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, and NaBH₄; acetylation, formylation, and oxidation, reduction; metabolic synthesis in the presence of tunicamycin; etc.

In addition, analogs and derivatives can be chemically synthesized. For example, a peptide corresponding to a portion of a polypeptide comprising a PDZ domain can be synthesized by use of a peptide synthesizer. Furthermore, if desired, nonclassical amino acids or chemical amino acid analogs can be introduced as a substitution or addition into the sequence. Non-classical amino acids include but are not limited to the D-isomers of the common amino acids, a-amino isobutyric acid, 4-aminobutyric acid, hydroxyproline, sarcosine, citrulline, cysteic acid, t-butylglycine, t-butylalanine, phenylglycine, cyclohexylalanine, α-alanine, designer amino acids such as β-methyl amino acids, Cα-methyl amino acids, and Nα-methyl amino acids.

### 5.7. Antibodies to Polypeptides Comprising a PDZ Domain

According to one embodiment, the invention provides antibodies and fragments containing the binding domain thereof, directed against polypeptides comprising a PDZ domain. Accordingly, polypeptides comprising a PDZ domain, fragments, analogs, or derivatives thereof, in particular, may be used as immunogens to generate antibodies against such polypeptides, fragments, analogs, or derivatives. Such antibodies can be polyclonal, monoclonal, chimeric, single chain, Fab fragments, or from an Fab expression library. In a specific embodiment, antibodies specific to the PDZ domain of a polypeptide comprising a PDZ domain may be prepared.

Various procedures known in the art may be used for the production of polyclonal antibodies. In a particular embodiment, rabbit polyclonal antibodies to an epitope of a polypeptide comprising a PDZ domain, or a subsequence thereof, can be obtained. For the production of antibody, various host animals can be immunized by injection with the native polypeptide comprising a PDZ domain, or a synthetic version, or fragment thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96) may be used.

Antibody fragments which contain the idiotype (binding domain) of the molecule can be generated by known techniques. For example, such fragments, include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g. ELISA (enzyme-linked immunosorbent assay).

### 6. EXAMPLES

### 6.1. Identification of Genes Encoding PDZ Domains from cDNA Expression Libraries Using Recognition Units Derived from Know Receptors

PDZ domains have been observed to bind with high specificity to certain proteins that contain at their carboxyl terminus the consensus sequence Xaa-(Ser/Thr)-Xaa-Val-COOH (SEQ ID No:4), where Xaa can be any amino acid. A study was initiated to identify novel PDZ recognition units and novel PDZ domain containing proteins using as recognition unit probes, short polypeptides corresponding to known proteins which contain this consensus sequence or a slight modification thereof, at the carboxyl terminus. Such “functional” screens for PDZ domain containing proteins were not previously known and in the absence of the screening methodology disclosed herein, are difficult to develop due to the low degree of sequence homology among PDZ domain-containing proteins. Thus, for example, an oligonucleotide probe could not be designed with any degree of confidence based on the low degree of homology of primary sequences of PDZ domains.

Potential peptide ligands were derived from a database search using osite from the Swiss Protein database, to identify proteins encoding either the PDZ C-terminal domain-binding consensus motif Xaa-Ser/Thr-Xaa-Val-COOH (SEQ ID NO:4) or the expanded PDZ consensus motif Xaa-Ser/Thr-Xaa-Yaa-COOH (SEQ ID NO:82), where Xaa can be any amino acid and Yaa is a small hydrophobic C-terminal amino acid. The database search revealed dozens of proteins, mostly receptors, channel proteins and viral proteins containing the consensus motifs.

Synthetic peptides that contained the consensus C-terminal PDZ binding motif from a wide variety of different proteins identified in the database search were synthesized (FIGS. 5A and 5B) using techniques known in the art. Merrifield, 1964, J. Am. Chem. Soc. 85:2149; Vale et al., 1981, Science 213:1394-1397; Marki et al., 1981, J. Am. Chem. Soc. 103:3178 and in U.S. Pat. Nos. 4,305,872 and 4,316,891. Briefly, solid phase peptide synthesis was performed on an Applied Biosystems Inc. (“ABI”) model 431A automated peptide synthesizer using the “Fastmoc” synthesis protocol supplied by ABI, which uses 2-(1H-Benzotriazol-1-yl)-1,1,3,3,-tetramethyluronium hexafluorophosphate (“HBTU”) (R. Knorr et al., 1989, Tet. Lett. 30:1927) as coupling agent. The peptides consisted of the 12 carboxyl terminal amino acids of the selected protein and were synthesized with either an N-terminal biotin-Ser-Gly-Ser-Gly (SEQ ID NO:89) or biotin-Lys-Gly-Lys-Gly (SEQ ID NO:90) linker. The peptides were purified by HPLC and their structure confirmed by mass-spectroscopy and amino acid analysis. Multivalent peptide-streptavidin/alkaline phosphatase probe complexes were assembled as described in Sparks et al. (1996, Proc. Natl. Acad. Sci. USA 93:1540-1544).

λ-cDNA expression libraries generated from LNCAP human prostate cell line (λgt22a human prostate cell line) mRNA using techniques known in the art, human brain mRNA (Clontech, San Diego, Calif.), human heart mRNA, human colorectal adenocarcinoma cell line mRNA (Clontech, San Diego, Calif.), human pituitary mRNA (Clontech, San Diego, Calif.), and human spinal cord mRNA (Clontech, San Diego, Calif.) were screened with equimolar concentrations of four or more different multivalent peptide-streptavidin/alkaline phosphatase probe complexes.

Screening of the libraries, including biotinylation of the peptide recognition units and their complexation with streptavidin-alkaline phosphatase, was as follows.

The λ cDNA expression libraries were plated at a density of 1×10⁵ pfu per plate. After 6 hours incubation at 370C, a nitrocellulose filter soaked in 10 mM isopropyl-β-D-thiogalactopyranoside (IPTG) was overlaid on each plate and incubated 3-6 hours at 37° C. Before the filters were removed from the plates, they were marked asymmetrically with India ink in a 18 gauge syringe needle. The plates were stored at 4° C. until ready for the secondary screen. The filters were washed with PBS (137 mM NaCl, 2.7 mM KCl, 4.3 mM Na₂HPO₄, 1.4 mM KH₂PO₄)-0.05% Triton X-100 three times at room temperature, 15 minutes each wash, and then placed in a plastic bag containing non-specific blocking solution (PBS-2% BSA) for one hour. In the meantime, 1 ml of 1 mM biotinylated peptide in PBS-0.1% Tween 20 was added to 20 ml of 1 mg/ml streptavidin-alkaline phosphatase (SA-AP) in PBS-0.1% Tween 20 and incubated at 4° C. for 30 minutes. As an alternative method of forming multivalent complexes, 50 pmol biotinylated peptide could have been incubated with 2 μg SA-AP (for a biotin:biotin-binding site ratio of 1:1). Excess biotin-binding sites would then be blocked by addition of 500 pmol biotin. As a further alternative, 31.2 μl of 1 mg/ml SA-AP could have been incubated with 15 μl of 0.1 mM biotinylated peptide for 30 min at 4° C. Ten μl of 0.1 mM biotin would then be added, and the solution incubated for an additional 15 min.

The preconjugated peptide recognition unit was introduced into the plastic bag containing the nitrocellulose filters and incubated overnight at room temperature. After three washes with PBS-0.1% Tween 20, the filters were incubated in 50 ml of 50 mg/ml 5-bromo-4-chloro-3-indolyl phosphate (BCIP), 100 ml of 50 mg/ml of dimethylformamide (DMF), and 15 ml of alkaline phosphatase buffer (0.1 M Tris-HCl, pH 9.4, 0.1 M NaCl, 50 mM MgCl₂). Strong positive signals were evident in 5-10 minutes.

Positive plaques were cored with a Pasteur pipet from the petri plates that had been spread with the full cDNA library and left in 500 μl of SM for 1 hour at room temperature or overnight at 4° C. with a drop of chloroform present. Five microliters of a 1:100 dilution of the eluted phage were plated out for rescreening, with the intention of reducing the number of plaque forming units (pfu) by a factor of 10 (i.e. 1×10⁵ in the primary screen, 3×10³ in the secondary, etc.), until all the plaques were positive when screened. To evaluate the size of the cDNA inserts in each plasmid, approximately 1/20 of each purified DNA sample was digested with EcoRI and HindIII to release the insert and resolved by agarose gel electrophoresis. DNA was sequenced by the dideoxy method with the T7 gene 10 oligonucleotide primer.

Thirty three clones were identified and isolated when the cDNA libraries were screened with the mixture of preconjugated biotinylated recognition units. A summary of the results obtained from the screening of the expression libraries is presented in Table 8. The cDNA inserts of the clones were sequenced on both strands using ABI PRISM TM dye terminator cycle chemistry (Perkin/Elmer) on an ABI 373A automated DNA sequencer.

Table 8 shows the human PDZ domain-containing proteins isolated according to the methods of the present invention. Mixtures of equimolar ratios of biotinylated potential PDZ domain peptide ligands recognition units, as defined in FIG. 5A and 5B, were used to screen cDNA expression libraries generated from LNCAP human prostate cancer cell line (λgt22a) mRNA, human brain mRNA human heart mRNA, colorectal adenocracinoma cell line mRNA, pituitary mRNA, and spinal cord mRNA (λgt11). The column labelled “Peptides” lists the recognition unit probe mixture (corresponding to those presented in FIGS. 5A and 5B) that was observed to bind to clones encoding each respective PDZ domain containing protein. In cases where the protein is known, the Genbank Accession number of the protein is also provided (column 5).

TABLE 8
PDZ domain-containing proteins isolated using COLT.

| | | | | | Number |
|---|---|---|---|---|---|
| | | Clone | | GeneBank | of |
| Library | Peptides | number | Identity | Accession # | clones |
| Brain | 2, 3, 5, 7 | 86 | Chapsyn | U49049 | 6 |
| | | 92 | PSD-95 | U32376 | 4 |
| | | 95 | Hdlg-1 | U13897 | 2 |
| LNCAP | 1, 2, 3, | 101 | Hdlg-1 | U13897 | 2 |
| | 4, 5, 6, | 103 | Novel PDZP1 | | 1 |
| | 7, 8, 9, 10 | 104 | Novel PDZP2 | | 1 |
| LNCAP | 27, 32, | 134 | NHE-RF | U19815 | 2 |
| | 33, 34 | 136 | TKA-1 | Z50150 | 4 |
| | | 138 | K1AA-147 | D63481 | 3 |
| | | 139 | SIP-1 | U82108 | 1 |
| | | 143 | Novel PDZP3 | | 1 |
| | | 150 | E3KARP | AF004900 | 2 |
| Heart | 1, 9, | 216 | Novel PDZP2 | | 1 |
| Colorectal | 31, 34 | 39 | Novel PDZPZ | | 1 |
| Adeno- | 25, 26 | | | | |
| carcinoma | | | | | |
| Pituitary | 15, 16, | 523 | Novel PDZP5 | | 1 |
| | 17, 18, 6, | 685 | Novel PDZP5 | | 1 |
| | 7, 8, 9 | | | | |
| Spinal | 34, 41, | 625 | Novel PDZP1 | | 1 |
| Cord | N1, N3 | 629 | Novel PDZP5 | | 1 |
| | | 630 | Novel PDZP1 | | 1 |

The expression library screens identified five novel human PDZ domain encoding genes in addition to the functional identification of four PDZ domains from KIAA-147, a previous reported protein of unknown function (Nagase et al., 1995 DNA Research 2:167-174). We demonstrate that the novel PDZ domains contained in these proteins bind distinct PDZ motif peptide ligands derived from a variety of signaling or regulatory proteins with differential specificity and relative affinity. In addition, we demonstrate that peptides containing C-terminal PDZ domain-binding motifs derived from a wide variety of receptors can bind to several novel and known PDZ domains.

Sequence analysis revealed that all the clones that were isolated during the screen encoded at least one PDZ domain. In some cases several siblings were identified derived from the same mRNA. From the brain λ-cDNA library we isolated partial cDNAs of the genes: Chapsyn, PSD-95 and hdlg-1. One clone corresponded to the full-length cDNA of Chapsyn. Chapsyn (Kim et al., 1996 Neuron 17:103-117) and PSD-95 (Cho et al., 1992, Neuron 9:929-942) are well known members of PDZ containing proteins from the postsynaptic density membrane of neurons. hdlg-1 is the human homolog of the Drosophila protein discs large (Lue et al., 1994, Poc. Natl. Acad. Sci. USA 91:9818-9822) and has been shown to function as a tumor suppressor protein. Chapsyn, PSD-95 and hdlg-1 form part of a subclass of the MAGUK (membrane-associated quanylate kinase) superfamily of proteins that share a common protein structure consisting of three PDZ domains followed by an SH3 domain and a domain homologous to yeast guanylate kinase.

From the LNCAP library, several members belonging possibly to a protein family of PDZ containing proteins were isolated. These were PDZP-134 and 150, which correspond to NHE-RF and E3KARP, two protein co-factors that regulate the renal brush border membrane Na⁺-H⁺ exchanger (Weinman et al., 1995, J. Clin. Invest. 95:2143-2149; Chris et al., 1997, Proc. Natl. Acad. Sci. USA 94:3010-3015); PDZP-139 corresponding to SIP-1, a nuclear factor that hinds to SRY a human testis determining factor (Poulat et al., 1997, J. Biol. Chem. 7167-7172); and PDZP-136, corresponding to TKA-1, a tyrosine activator protein that activates the platelet-derived growth factor receptor (unpublished). The PDZ domains of these regulatory proteins contain carboxylate-binding loop having the sequence Gly-Tyr-Gly-Phe (SEQ ID NO:91) as a characteristic feature, a variation of the Gly-Leu-Gly-Phe (SEQ ID NO:3) found in other PDZ domains, such as, for example, PSD-95 and hdlg. The PDZ domains of SIP-1 and TKA-1 interact with proteins that contain at their C-terminus the extended PDZ C-terminal consensus sequence Xaa-Ser/Thr-Xaa-Leu (SEQ ID NO:92). The cDNA clones, PDZP-134, PDZP-136, PDZP-139 and PDZP-150 were isolated with a mix of peptides that included two biotinylated peptides ending at the C-terminus with a leucine (FIG. 5A, peptides 32 and 33 in rows 41 and 42).

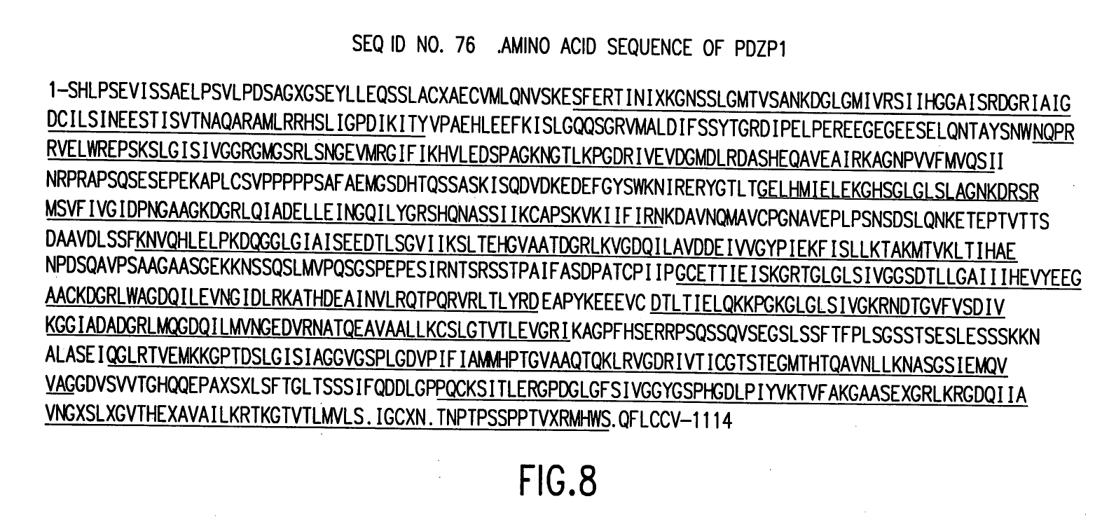

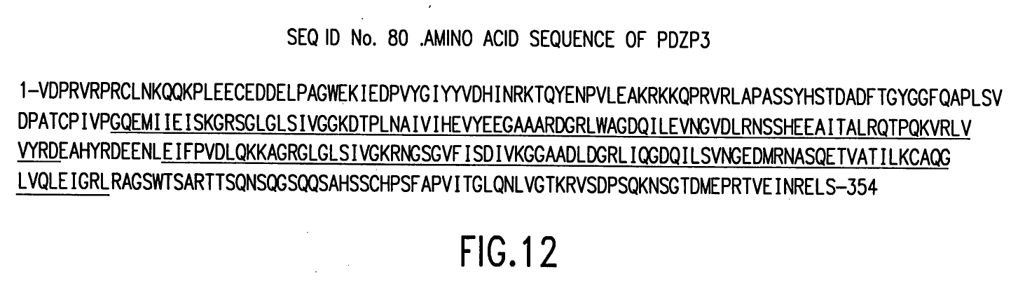

Two of the novel clones isolated from the LNCAP library, PDZP1 (FIGS. 7A, 7B, and 8) and PDZP2 (FIGS. 11 and 12), contain 8 and 4 PDZ domains, respectively, showing some homology to existing PDZ domains (FIGS. 3A and 3B). The nucleotide sequence of PDZP1 is presented in FIGS. 7A and 7B. An expressed sequence tag (EST) from human (EST20397) was identified that is 98% identical to PDZP1 as depicted in FIGS. 7A and 7B. The PDZ domains of PDZP1 span amino acid residues 50-134 (SEQ ID NO:111), 193-288 (SEQ ID NO:112), 359-445 (SEQ ID NO:10), 492-576 (SEQ ID NO:11), 638-724 (SEQ ID NO:113), 735-819 (SEQ ID NO:114), 871-960 (SEQ ID NO:115) and 996-1083 (SEQ ID NO:116) as depicted in FIG. 8 and are encoded by nucleotides 150-404 (SEQ ID NO:120); 579-866 (SEQ ID NO:121); 1077-1337 (SEQ ID NO:122); 1476-1732 (SEQ ID NO:123); 1914-2174 (SEQ ID NO:124); 2205-2461 (SEQ ID NO:125); 2613-2678 (SEQ ID NO:126); 3006-3270 (SEQ ID NO:127) as depicted in FIGS. 7A and 7B, respectively. The nucleotide sequence of PDZP2 is presented in FIG. 9. The PDZ domains of PDZP2 span amino acid residues 134-219 (SEQ ID NO:12); 305-386 (SEQ ID NO:13); 475-559 (SEQ ID NO:14); and 632-730 (SEQ ID NO:15) as depicted in FIG. 10. The nucleotide sequences encoding the PDZP2 PDZ domains span nucleotides 359-655 (SEQ ID NO:128); 911-1156 (SEQ ID NO:129); 1421-1678 (SEQ ID NO:130); and 1892-2188 (SEQ ID NO:131) as depicted in FIG. 9.

Screening of the LNCAP library also resulted in the isolation of clone PDZP-104. To our surprise the nucleotide sequence of clone PDZP-104 showed 100% homology at the 5′ end to that of a clone encoding a human WW domain containing protein fragment (U96115) that was previously isolated in our laboratory and reported (Pirozzi et al., 1997, J. Biol. Chem. 272:14611-14616). The cDNA clone encoding this WW domain containing protein fragment and the cDNA clone encoding the PDZP-104 protein fragment therefore form part of the same gene, which encodes a protein we have named PDZP2. Screening of the human heart and colorectal adenocarcinoma cDNA libraries identified two additional overlapping clones encoding PDZP2. Clone PDZP-216 was isolated from the human heart, expression library (Clontech, San Diego, Calif.) and PDZP-39 was isolated from the colorectal adenocarcinoma cDNA expression library (Clontech, San Diego, Calif.). Clone PDZP-216 overlaps with both the clone encoding the WW fragment and clone PDZP-104. Clone PDZP-39 overlaps with clone PDZP-104.

The “complete” (missing the amino terminus) amino acid sequence of PDZP2 generated by piecing together the nucleic acid sequence of the overlapping clones is presented in FIG. 10. Interestingly, clone PDZP-39 encodes two additional PDZ domains extending the total number of PDZ domains in PDZP2 to four (FIG. 10). Additionally, further, sequence analysis of the WW domain encoding clone revealed that the protein fragment encoded by this clone has an additional, previously unidentified, WW domain which spans amino acid residues 23-60 as depicted in FIG. 10, and is encoded by nucleotides 67-120 as depicted in FIG. 9. Surprisingly, PDZP2 contains both WW and PDZ domains. This is the first instance that PDZ domains and WW domains have been found in association on the same protein. Intriguing is also the presence of a 21 amino acid polyglutamine (Poly Q) stretch between one of the WW domains and one of the PDZ domains of PDZP2 (FIG. 10). Recently, it has been shown that polyglutamine stretches may be also involved in specific protein-protein interactions (Burke et al., 1996, Nature Med. 2:347-350). The guanylate-like kinase (GK) domain found at the amino terminus of PDZP2 may also act as a site for protein-protein interactions (Takeuchi et al., 1997, J. Biol. Chem. 272:11943-11951; Kim et al., 1997, J. Cell. Biol. 136:669-678). The novel PDZP2 protein seems therefore to consist of multiple protein-protein interaction domains. The presence of multiple protein domains (PDZ, WW, PolyQ and GK) within the same protein indicates that this novel protein may function as a scaffold protein to link various components together to form a multiprotein complex.

Screening of the pituitary expression library yielded clone, PDZP4, which encodes four novel PDZ domains and two novel WW domains. The nucleic acid sequence of PDZP4 is disclosed in FIG. 13. The PDZ domains of PDZP4 span amino acid residues 207-292 (SEQ ID NO:18), 386-465 (SEQ ID NO:19), 545-630 (SEQ ID NO:20) and 688-780 (SEQ ID NO:21) as depicted in FIG. 14; and are encoded by nucleotides 548-798 (SEQ ID NO:136); 1157-1396 (SEQ ID NO:137); 1634-1891 (SEQ ID NO:138); 2063-2341 (SEQ ID NO:139) as depicted in FIG. 13, respectively. The WW domains of PDZP4 span amino acid residues 87-124 (SEQ ID NO:142) and 133-170 (SEQ ID NO:143) as depicted in FIG. 14; and are encoded by nucleotides 259-372 (SEQ ID NO:140) and 397-510 (SEQ ID NO:141), as depicted in FIG. 13, respectively.

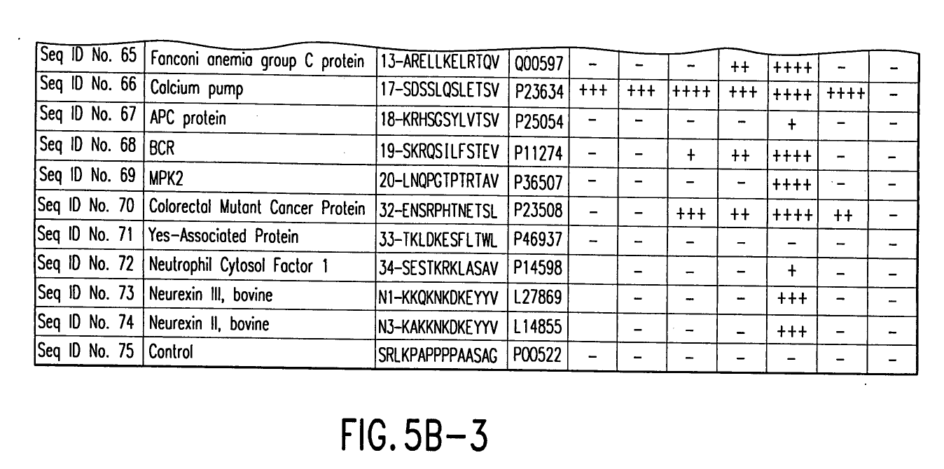

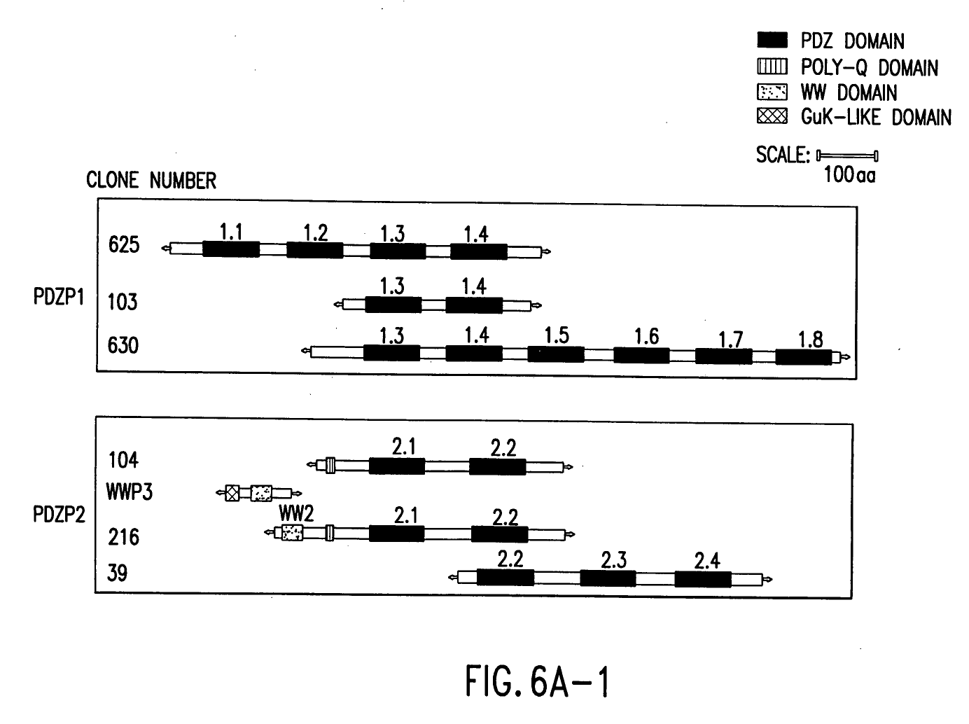

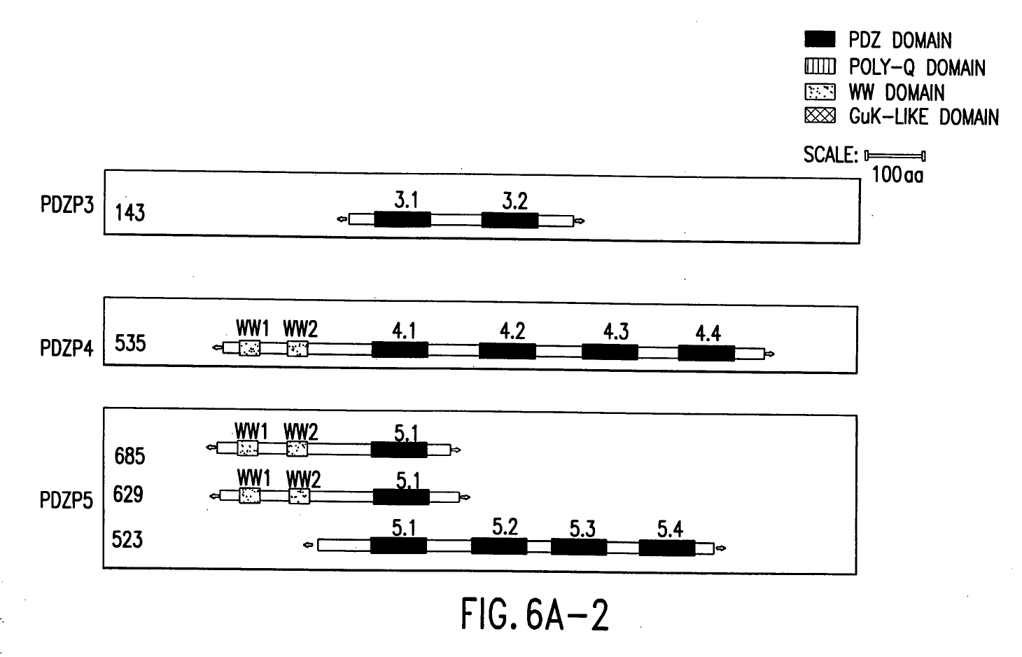

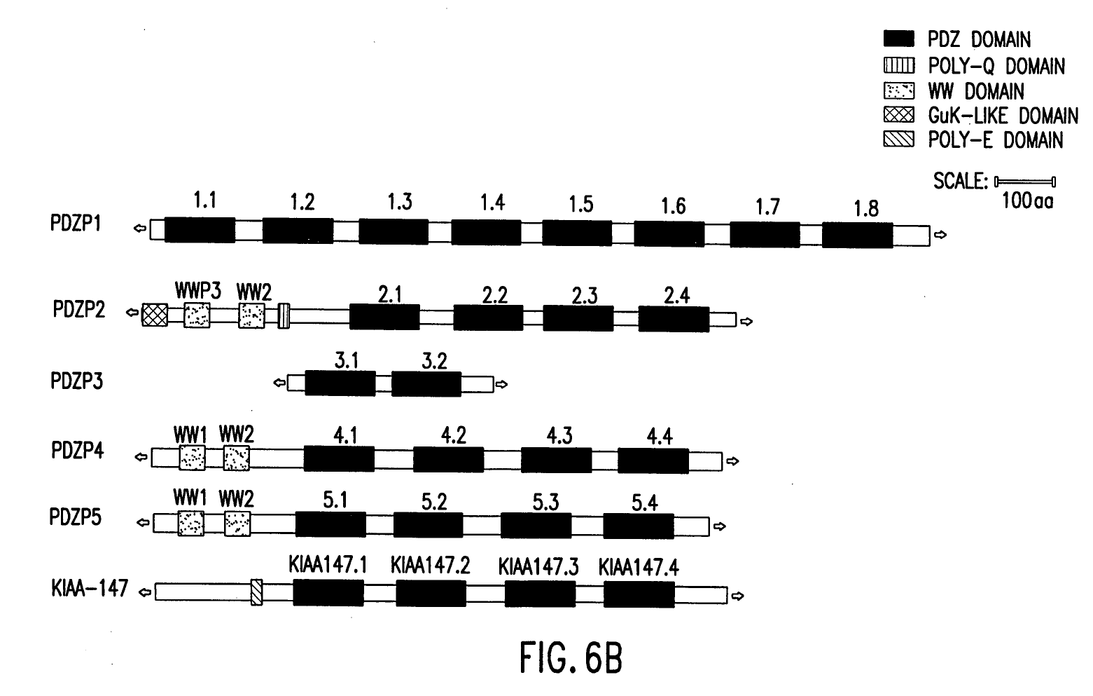

Screening of the spinal cord expression library identified clone PDZP5, which encodes four novel PDZ domains. Interestingly, PDZP5, like PDZP2, also encodes two WW domains (FIG. 6B). The PDZ domains of PDZP5 span amino acid residues 248-333 (SEQ ID NO:22), 416-495 (SEQ ID NO:23), 564-649 (SEQ ID NO:117), and 690-779 (SEQ ID NO:118), as depicted in FIG. 16; and are encoded by nucleotides 742-999 (SEQ ID NO:144), 1246-1480 (SEQ ID NO:145), 1507-1947 (SEQ ID NO:146), and 2068-2337 (SEQ ID NO:147), as depicted in FIG. 15, respectively. The WW domains of PDZP5 span amino acid residues 141-166 and 187-212 as depicted in FIG. 16 and are encoded by nucleotides 421-498 (SEQ ID NO:148) and 599-630 (SEQ ID NO:149) as depicted in FIG. 15, respectively.

An alignment of the twenty two novel PDZ domains along with the third PDZ domain of PSD-95 is shown in FIGS. 3A and 3B. The overall amino acid homology between the novel PDZ domains and the third PDZ domain of PSD-95 ranges between 30% and 40%. Certain features are characteristic of PDZ domains as shown in the novel PDZ domains. The positively charged amino acid, lysine or arginine, corresponding to position Arg-318 of the third PZD domain of PSD-95 and the two glycines that form part of the carboxylate-binding loop are conserved in all PDZ domains. Interestingly, all eighteen novel PDZ domains contain variations of the carboxylate-binding loop (Gly-Leu-Gly-Phe) (SEQ ID NO:3) found within PDZ domains of proteins such as PSD-95, hdlg-1 and others. This amino acid sequence is found in PDZP1.3 and KIAA-147.2. The amino acid sequence Gly-Leu-Gly-Leu (SEQ ID NO:93) is found in the PDZ domains PDZP1.3, PDZ3.1 and PDZ 3.2. The amino acid sequence Gly-Leu-Gly-Ile (SEQ ID NO:94) is found in the PDZ domains of PDZP1.2, PDZP1.4, PDZP1.5, PDZP1.6 and KIAA-147.1. All four PDZ domains of PDZP2, PDZP4, and PDZP5 have the sequence Gly-Phe-Gly-Phe (SEQ ID NO:95) (FIG. 4). These combinations of motifs for the hydrophobic pocket of PDZ domains with the exception of Gly-Leu-Gly-Ile (SEQ ID NO:96) have not been previously described for human PDZ containing proteins. Of interest is also the substitution of the conserved histidine (His-372 in PSD-95) to isoleucine in PDZP1.4 and glutamine in PDZP3.2 and PSD1.6. His-372 of the third PDZ domain of PSD-95 is an important residue that is part of the carboxylate-binding loop of the PDZ domain and has been shown in the crystal structure to form hydrogen bonds with threonine, the second amino acid of the C-terminal PDZ peptide ligand (Doyle et al., 1996, Cell 85:1067-1076). These changes in the carboxylate-binding loop may confer different binding characteristics to the novel PDZ domains.

Of special interest is PDZP-138 that matched an entry in the Genbank database, KIAA-147 (Genbank Acc. # D63481). This gene was previously isolated as part of a project that identified 40 new genes from a human cell line KG-1 (Nagase et al., 1995, DNA Research 2:167-174). KIAA-147 contains two interesting features: homology to adenyl cyclase at the amino terminus and a stretch of glutamines. In addition, we show here that KIAA-147 also contains four functional PDZ domains that bind to various peptide ligands containing the PDZ C-terminal consensus sequence. The homologies of the first and second PDZ domains of KIAA-147 with the third PDZ domain of PSD-95 is 40% and 37%, respectively. The function of this gene is not known, but due to these features, it is likely to be involved in signal transduction.

### 6.2. Cross Affinity Mapping

To determine the ligand preferences of the novel PDZ domain-containing clones described in Section 6.1 the novel PDZ domains derived from PDZP2, PDZP3, KIAA-147, and the three PDZ domains of PSD-95 and Chapsyn were subcloned into glutathione S-transferase (GST) expression vectors and expressed as GST fusion proteins. We examined the peptide ligand binding preferences of all sixteen individual PDZ domains in an enzyme-linked immunosorbent assay-based cross-affinity map experiment. As mentioned above, 45 peptides were chosen from database searches and grouped into seven classes (G-protein coupled receptors, viral proteins, glutamate receptors, ion channels, Frizzled homologs, transporters and various other proteins). We tested the ability of these peptides to bind to the known and novel PDZ domains.

PCR fragments encoding individual PDZ domains were subcloned into the Sal1 and Notl sites of pGEX-4T-2 (Pharmacia Biotech, Inc.) and fusion proteins were expressed and purified as described by the manufacturer. ELISA based cross-affinity experiments were performed essentially as described by Sparks et al. (1996, Proc. Natl. Acad. Sci. USA 93:1540-1544) with the following modifications. Briefly, microtiter wells were coated with 1-5 pg of fusion protein in 100 mM NaHCO₃, blocked with SuperBlock TBS (Pierce) and washed four times with PBS, 0.05% Tween 20. Specific peptide-streptavidin/alkaline phosphatase complexes were added as above and unbound complexes washed five times with PBS, 0.05% Tween 20. Following addition of PNP substrate (p-nitrophenyl phosphate, Kirkegard & Perry Labs), peptide binding was quantitated after 30 min. at O.D. 405 nm. Relative binding measurements from three independent determinations were assigned to a scale as follows: O.D. units 0−0.5=(−), 0.5−1.0=(+), 1.0−2.0=(++), 2.0−3.0=(+++)>3.0=(++++). Peptide sequences used in cross-affinity experiments correspond to C-terminal segments of the following proteins: protein name (species: H=Human; M=Mouse; R=Rat; B=Bovine; V=Viral Origin, Genbank accession number); β1-adrenoreceptor (H, P18090); Serotonin Receptor (H, P28223); VIP Receptor (H, P32241); CRF Receptor (H, P34998); Orphan Receptor (H, P46089); β-1 Adrenergic Receptor (H, P08588); COM (ADE02, P03267); E6, HPV18 (V, P06463); UL25, HSV11 (V, P10209); GP3, EBV (V, P03200); TAT, HTL1A (V, P03409); UL14, VZVD (V, P09295); NMDA Receptor, NR2B (M, Q01097); NMDA Receptor subunit (H, U08266); mGluR1α (H, U31215); mGluR5a (H, D28538); mGluR (H, L76631); mGluR3 (H, AC002081); AMPA receptor (H, L20814); K⁺-Channel, KV 1.4 (H, P22459); K⁺-Channel Kir 2.2v (H, U53143); Na⁺-Channel (a) (H, P15389); K⁺-Channel (Kir) (H, D50582); Transmembrane Receptor (Homolog of frizzled ) (H, U43318); Homolog of frizzled (R, L02529); Homolog of frizzled (M, U43319); Glucose transporter (H, P11166); Excitatory Amino Acid Transporter (H, P43003); FAS Receptor (H, P25445); NGF Receptor (H, P08138); Neuropeptide Y Receptor, type 2 (H, P49146); Somatostatin Receptor, type 2 (H, P30874); CFTR (H, P13569); V-CAM (H, P19320); Ankyrin (H, Q01484); Fanconi anemia group C protein (H , Q00597); Calcium pump (H, P23634); APC protein (H, P25054); BCR, (H, P11274); MPK2 (H, P36507); Colorectal Mutant Cancer Protein (H, P23508); 65 KD Yes-Associated Protein (H, P46937); Neutrophil Cytosol Factor 1 (H, P14598); Neurexin III, (B, L27869); Neurexin II (B, L14855). Protein sequence homology searches were performed using BLAST (Altschul et al., 1990, J. Mol. Biol. 215:403-410).

The results of the cross-affinity map experiment is summarized in FIGS. 8A and 8B. In general, the majority of the peptides showed differences in specificity and relative binding to the PDZ domains examined in this study. Also not all the peptides displayed an ability to bind to the PDZ domains even though they contained the PDZ domain-binding consensus sequence motif. It is also apparent from our cross-affinity map that the individual PDZ domains are able to interact with more than one peptide ligand. The results of the cross-affinity map show that some PDZ domains bind with broad specificity to the peptides in this study while other PDZ domains have a more restricted pattern of interaction. It has been previously reported that the first and second PDZ domain of PSD-95 but not the third PDZ domain can interact specifically with Shaker K⁺-channels and NMDA receptor subunits of the NR2 subfamily (Niethammer et al., 1996, J. Neurosci. 16:2157-2163). These results are confirmed in our in vitro assay (FIG. 8, rows 13 and 20) where the peptides corresponding to the NMDA receptor NR2 subunit and K⁺-channel bind to the first and second PDZ domain but not the third PDZ domain of PSD-95. Interestingly, the three PDZ domains of Chapsyn, a close relative of PSD-95, show the same binding specificities for the NMDA receptor NR2 subunit and K⁺-channel peptide ligands, but a different binding pattern with the other peptide ligands in FIGS. 8A and 8B. Of interest is also the observation that a human NMDA subunit (FIG. 8B, row 14) bound with high affinity only to the second PDZ domain of PSD-95. This interaction may be significant in vivo and may further add another binding partner to PSD-95. None of the other selected receptors from the glutamate group bound to any of the twelve PDZ domains. These glutamate receptors (FIGS. 8A and 8B, rows 15 to 19) contain either leucine or isoleucine at the carboxyl end. Recently, two novel PDZ containing proteins, Grip and Homer, were identified that specifically interact with the C-termini of AMPA receptors and metabotrobic glutamate receptors which contain a PDZ C-terminal consensus motif ending in leucine or isoleucine (Dong et al., 1997, Nature 386:279-283; Brakeman et al., 1997, Nature 386:284-288).

Striking is also the result of the interactions of several viral proteins with the twelve PDZ domains in this study (FIGS. 5A and 5B). We tested six peptides derived from proteins of several viruses that contained the PDZ domain-binding C-terminal consensus motif Xaa-Ser/Thr-Xaa-Val-COOH (SEQ ID NO:4) which were three viral coat proteins (ADEO2, COM (SEQ ID NO:36)); HSV11, UL25 (SEQ ID NO:38); EBV, GP3 (SEQ ID NO:39), one viral transforming protein (HPV18, E6 (SEQ ID NO:37)), one transcriptional regulatory protein (HTL1A, TAT (SEQ ID NO:40) and one viral protein of unknown function (VZVD, UL14 (SEQ ID NO:41)). The ADE02 peptide bound specifically to only one of the novel PDZ domains (143-A). The rest of the five peptides displayed the ability to bind to the novel PDZ domains as well as to the PDZ domains of Chapsyn and PSD-95 to varying degrees FIGS. 5A and 5B). In this context, Lee et al. (1997, Proc. Natl. Acad. Sci. USA 94:6670-6675) show that viral oncoproteins that possess a consensus C-terminal PDZ domain-binding motif (Tax from HTLV-1 and E6 type 18 from HPVs) are able to bind in vitro to hdlg, the mammalian homolog of the Drosophila discs large tumor suppressor protein. In our cross-affinity map, the HPV18 peptide specifically binds also to the novel PDZ domains PDZP2.1, PDZP2.2, KIAA-147.1 and KIAA-147.2 as well as to the three PDZ domains of Chapsyn and the two first PDZ domains of PSD-95. This result suggests that the E6 viral oncoprotein could potentially interact also with various other host proteins and further contributing to the transforming potential of the viral proteins. Of interest is also the observation that peptides derived from the C-terminus of viral coat proteins containing a PDZ C-terminal consensus sequence interact with several of the novel PDZ domains as well as the PDZ domains of PSD-95 and Chapsyn (FIG. 5B, rows 7, 9 and 10). The results of these interactions suggest a mechanism for selected viruses to use host proteins containing PDZ domains for viral assembly and budding. In support of this hypothesis, there are indications that cytoskeletal host proteins containing WW domains may interact with the retroviral coat protein (Gag) and that these interactions may be essential in the assembly and budding of the retroviruses (Garnier et al., 1996, Nature 381:744-745). In addition, most of the PDZ containing proteins that have been so far described localize at the plasma membrane where viral assembly and budding occurs. Modular protein domains like PDZ and WW domains may play therefore a critical role in the different stages of certain viral life cycles where protein-protein interactions between the viral proteins (transcription factors, coat proteins, oncoproteins) and host proteins are needed for the propagation of the virus.

Of interest is also the results obtained examining the interactions of the two peptides derived from the APC (Adenomatous polyposis coli (SEQ ID NO:67)) and MCC (Mutated Colon Cancer (SEQ ID NO:70)) protein with the twelve PDZ domains assayed. The MCC gene is tightly linked to the APC locus and mutations in both genes have been implicated in colon tumor formation (Bonneton et al., 1996, C. R. Acad. Sci. III 319:861-869). The APC gene may play a role in both hereditary and nonhereditary cancers of the colon while the MCC gene is apparently involved only in the nonhereditary type. The APC protein was shown to bind to hdlg, the human homolog of the Drosophila discs large tumor suppressor protein (Matsumine et al., 1996, Science 272:1020-1023). This interaction requires the carboxyl-terminal region of APC and the first two PDZ domains of hdlg (Lue et al., 1994, Proc. Natl. Acad. Sci. USA 9818-9822). In our cross-affinity map the peptide corresponding to the APC protein interacts only weakly with four of the PDZ domains (FIGS. 5A and 5B). In contrast, the peptide corresponding to the MCC protein which contains the C-terminal PDZ consensus motif Glu-Thr-Ser-Leu (SEQ ID NO: ______) binds specifically to several PDZ domains (FIGS. 5A and 5B). This result suggests that the MCC protein, like the APC protein, may bind to a PDZ domain containing protein.

The results of the binding characteristics of the two PDZ domains of KIAA-147 (KIAA-147.1 and KIAA-147.2) against our panel of peptide ligands is shown in FIGS. 5A and 5B. The cross-affinity map reveals that KIAA-147.1 is less restrictive in its binding specificity than KIAA-147.2, as it may bind with high affinity to ligands containing other small hydrophobic amino acids, such as, alanine or leucine. An important difference between the two PDZ domains is the substitution of phenylalanine for isoleucine in the hydrophobic groove of KIAA-147.1. The crystal structure of PDZ domain 3 of PSD-95 complexed with the peptide ligand (-Gln-Thr-Ser-Val-COOH) (SEQ ID NC:97) reveals that Phe-325 forms hydrogen bonds not only with the carboxyl terminus of the peptide ligand but also with valine (Doyle et al., 1996, Cell 85:1067-1076). These interactions are important for the specificity of the binding of PDZ domain and ligand. The presence of isoleucine instead of phenylalanine at this position of KIAA-147.1 might therefore change the characteristics of the PDZ domain and allow for binding of ligands with other small hydrophobic amino acids.

Several other interactions derived from the cross-affinity map between the peptides and the novel and known PDZ domains are of interest. For example, various G-protein coupled receptors may potentially interact via their C-terminus to PDZ domain containing proteins (FIGS. 5A and 5B, SEQ ID NOS:31, 32, 33, 34 and 35). The peptide derived from the calcium pump protein (SEQ ID NO:66) binds to several PDZ domains suggesting a mechanism for anchoring these proteins in the plasma membrane. The peptides derived from V-CAM (SEQ ID NO:63) and NGF (SEQ ID NO:59) bind with high affinity to the first domain of the novel clone PDZP3. Of interest are also the interactions of the peptides derived from the Fanconi anemia group C protein (SEQ ID NO:65), BCR (SEQ ID NO:68), MPK2 (SEQ ID NO:69) with several PDZ domains in this study.

The positive interaction of peptides corresponding to the 12 carboxy terminal amino acids of: K+-Channel, KV 1.4 (SEQ ID NO:49); FAS Receptor (SEQ ID NO:58); NMDA (NR2B), mouse (SEQ ID NO:42); NGF Receptor (SEQ ID NO:59); β1 Adrenoreceptor (SEQ ID NO:30); Serotonin (SEQ ID NO:31); VIP (SEQ ID NO:32); CRF (SEQ ID NO:33); Na+ Channel (α) (SEQ ID NO:51); Orphan Receptor (SEQ ID NO:34); Ankyrin (SEQ ID NO:64); Fanconi anemia group C protein (SEQ ID NO:65); Glucose transporter (SEQ ID NO:56); β-1 Adrenergic (SEQ ID NO:35); Calcium pump (SEQ ID NO:66); BCR (SEQ ID NO:68); MPK2 (SEQ ID NO:69); HPV18, E6 (SEQ ID NO:37); HSV11, UL25 (SEQ ID NO:38); EBV, GP3 (SEQ ID NO:39); HTL1A, TAT (SEQ ID NO:40); VZVD, UL14 (SEQ ID NO:41); Somatostatin Receptor (Type2) (SEQ ID NO:61); Colorectal Mutant Cancer Protein (SEQ ID NO:70); Transmembrane Receptor (frizzled) (SEQ ID NO:53); Homologue of frizzled, rat (SEQ ID NO:54); Neurexin III, bovine (SEQ ID NO:73); and Neurexin II, bovine (SEQ ID NO:74) with the first and second PDZ domains of PSD-95 and peptides corresponding to the 12 carboxy terminal amino acids of: Calcium pump (SEQ ID NO:66); HSV11, UL25 (SEQ ID NO:38); Colorectal Mutant Cancer Protein (SEQ ID NO:70); Transmembrane Receptor (frizzled) (SEQ ID NO:53); and K+-Channel, Kir 2.2v (SEQ ID NO:50) with the third PDZ domain of PSD-95 (FIG. 8B) are of particular medical interest. The NMDA receptor functions as a glutamate-activated calcium channel. After a brain stroke, a surplus of glutamate is produced that causes an influx of calcium through the NMDA receptor. Calcium then activates NOS to produce nitric oxide through calmodulin. One of the major factors that causes brain damage in stroke is the accumulation of toxic levels of nitric oxide. Overstimulation of NOS is particularly efficient because NMDA receptors and NOS are linked together via the interaction between the second PDZ domain of PSD-95 and the single PDZ domain of NOS (Brenman et al., 1996, Cell 84:757-767; and Huang et al., 1994, Science 265:1883-1885).

A novel approach to avoid the excessive production of nitric oxide, without directly inhibiting the enzymatic activity of NOS, is to uncouple NOS from the NMDA receptor by finding a compound that inhibits the interaction of PSD-95 with NOS and/or the NMDA receptor. This strategy for avoiding excessive production of nitric oxide avoids some of the problems currently associated with some NMDAR antagonists or NOS inhibitors. Thus, membrane permeant compounds that block or interfere with association of nNOS with the second PDZ domain of PSD-95 and/or block the association of the PDZ domains of PSD-95 with NMDA receptors have great potential as therapeutics.

An inhibition assay essentially as described in Section 5.4 was performed to assess the ability of PDZ ligands to uncouple NOS from the NMDA receptor. This assay consisted of three components: (1) a biotinylated peptide known to interact with PSD95.1 (a peptide sequence corresponding to the 12 carboxy terminal residues of the Na⁺ channel cardiac receptor (SEQ ID NO:51), which was used to clone PSD-95 as described in Section 6.1); (2) test inhibitors (three inhibitors based on the biotinylated peptide were assayed; (a) an inhibitor corresponding to the 12 carboxyl terminal residues of the Na⁺ channel cardiac receptor (Biotin-Ser-Gly-Ser-Gly-Pro-Pro-Ser-Pro-Asp-Arg-Asp-Arg-Glu-Ser-Ile-Val-COOH; SEQ ID NO:157); (b) a dimer of the peptide sequence AC-Cys-Pro-Pro-Ser-Pro-Asp-Arg-Asp-Arg-Glu-Ser-Ile-Val-COOH cross-linked at the cysteine residue (a); (c) a 5mer containing the carboxy terminal 5 peptides of (a) (Arg-Glu-Ser-Ile-Val-COOH; SEQ ID NO:104); and (d) a scrambled version of inhibitor (c) (Ile-Ser-Val-Arg-Glu; SEQ IQ NO:105); and (3) a GST-PSD-95.1 fusion protein of the first PDZ domain of PSD-95 (generated according to the methods set forth in hereinabove). The assay was performed essentially as set forth infra. Particularly, biotinylated peptide and various concentrations of an inhibitor were added to microtiter plates coated with the GST-PSD-95.1 fusion protein and the binding interactions were quantified as described above. The results set forth in FIG. 22 demonstrate that the dimer inhibitor and to a lesser extent, the Arg-Glu-Ser-Ile-Val (SEQ ID NO:105) 5mer inhibitor, were able to inhibit the interaction of the PDZ domain and ligand. Thus, the peptides identified herein and other peptide binders of the PZD domains of PSD-95 and NOS, may be routinely identified according to the methods described herein, and assessed for their potential as therapeutics in the treatment and prevention of brain injury resulting from a stroke.

The assays described hereinabove may routinely be modified so as to assess the therapeutic potential of peptides to disrupt the interaction of other PDZ domains and their ligands. For example, the therapeutic potential of a peptide to treat or alleviate hypertension may be assessed by assaying the ability of the peptide to interfere with the interaction between the carboxy terminus of the Na⁺—H⁺ exchanger and a PDZ domain containing protein that binds the exchanger.

Additionally, the assay described above and the methods described herein may be routinely adapted to identify peptides that potentiate apoptosis by inhibiting or interfering with the interaction between the PDZ domain of PTPL1/FAP1 and the carboxy terminus of FAS. Further, these methods and assays may routinely be modified to identify peptides that alter cell growth by inhibiting or interfering with the interaction between the second PDZ domain of hdlg and the carboxyl terminus of APC.

Of a more general utility, mapping the interaction between PDZ domains and ligands for these domains is likely to lead to new insights into the possible function of novel and existing proteins by uncovering functional links between proteins that otherwise would not have been realized. This procedure also enables a large-scale analysis of the interactions of modular domain containing proteins and helps to build networks of protein-protein interactions. Ultimately, the knowledge gained from the understanding of these functional protein-protein interactions may be used to build a protein linkage map of the human proteome.

6.3. MATERIALS USED IN SECTION 6 AND ITS SUBSECTIONS

| 2xYT media (1L) | | |
|---|---|---|
| Bacto tryptone | 16 | g |
| Yeast Extract | 10 | g |
| NaCl | 5 | g |
| 2xYT agar plates | | |
| 2xYT + 15 g agar/L | | |
| 2xYT top agarose (8%) | | |
| 2xYT + 8 g agarose/L | | |
| SDS/DTT loading buffer | | |
| (10 mL of 5× solution) | | |
| .5 M Tris base | 0.61 | g |
| 8.5% SDS | 0.85 | g |
| 27.5% sucrose | 2.75 | g |
| 100 mM DTT | 0.154 | g |
| .03% Bromophenol Blue | 3.0 | mg |
| Overnight cell cultures: | | |
| Inoculate media with one isolated | | |
| colony of appropriate cell type and | | |
| incubate 37° C. O/N with shaking | | |
| BL21 (DE3) pLysE | | |
| 2xYT media | | |
| maltose | 0.2% | |
| MgSO₄ | 10 | mM |
| Chloramphenicol | 34 | μg/ml |
| Kanamycin | 50 | μg/ml |

### 6.4. Biotinylated Peptide Detection Using Tyramide Amplification System

The following protocol is an alternative to the methods described herein that utilize alkaline phosphatase to detect the binding of recognition units and PDZ domains. It permits the use of recognition units that are phosphopeptides.

Materials:

TSA-Tyramide Signal Amplification System (Dupont NEL-700); Streptavidin-Peroxidase, SA-P, conjugate 1 mg/ml H₂O (Sigma S-5512); Streptavidin-Alkaline Phosphatase, SA-AP, conjugate 1 mg/ml H₂O (Sigma S-2890); Dulbecco's PBS (Sigma D1408); PBS+0.05% Triton-X100, PBS/Tr; PBS/Tr+20%DMSO; SuperBlock™ Blocking Buffer in TBS (Pierce 37535); d-Biotin 0.1 mM; Biotinylated Peptide probe 0.1 mM; Plaque lifts on Nitrocellulose (Schleicher & Schuell BA85, 0.45 um, 85 mm); SIGMA FAST™ BCIP/NBT Buffered Substrate Tablets (Sigma B-5655)

Method:

1. Wash Plaque lifts in PBS/Tr ×5-10 min at Room Temperature (RT) with agitation.

2. Block filters in 50-75 ml SuperBlock at RT for 60-90 min or store at 4° C. until needed.

3. Prepare SA-P/biotinylated peptide probe complex while filters are in block.
Mix 93.6 μl SA-P 1 mg/ml and 45 μl 1.0 mM Biotinylated Peptide probe.
Incubate 30 min at 4° C.
Add 30 μl 0.1 mM d-Biotin and mix.
Incubate 15 min at 4° C.
Add above complex to 60 ml SuperBlock.

4. Add filters to SA-P/biotinylated peptide probe complex and incubate 2 hrs at RT with agitation.

5. Wash Plaque lifts in PBS/Tr 5×10 min at Room Temperature (RT) with agitation.

6. Place each filter in a petri dish and add 5ml Biotinyl Tyramide reagent prepared as follows;
Mix equal volumes of 2× amplification diluent and deionized water.
Add 40 μl Biotinyl Tyramide reagent/5 ml amplification diluent and mix.

7. Incubate Biotinyl Tyramide reagent on filters for 10 min at RT. Exposure time and concentration of Biotinyl Tyramide reagent of filters may have to be determined-empirically.

8. Wash filters thoroughly for:
4×10 min in 15 ml PBS/tr+20% DMSO.
3×5 min in 15 ml PBS/tr.
2×3 min in 10 ml SuperBlock.

9. Add filters to SA-AP diluted in SuperBlock (0.33 μl 1 mg/ml stock per 20 ml SuperBlock). Exposure time and concentration of SA-AP to filters may have to be determined empirically. Use about 10 ml per filter.

10. Incubate 30 min at RT.

11. Wash filters thoroughly for:
4×5 min in 15 ml PBS/tr.
3×5 min in PBS.

12. Develop filters using SIGMA FAST™BCIP/NBT Buffered Substrate Tablets. Use 60 ml for 10 filters.
Dissolve 1 tablet in 10 ml deionized water.
Allow development to proceed for 5-30 min at RT with agitation until desired signal to noise levels are visually obtained.
Rinse filters in water and air dry.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

1-78. (canceled)

79. A method of identifying a compound that affects the binding of a molecule comprising a PDZ domain and a recognition unit that selectively binds to the PDZ domain comprising:
(a) contacting the molecule comprising the PDZ domain and the recognition unit under conditions conducive to binding in the presence of a candidate compound and measuring the amount of binding between the molecule and the recognition unit;
(b) comparing the amount of binding in step (a) with the amount of binding known or determined to occur between the molecule and the recognition unit in the absence of the candidate compound, where a difference in the amount of binding between step (a) and the amount of binding known or determined to occur between the molecule and the recognition unit in the absence of the candidate compound indicates that the candidate compound is a compound that affects the binding of the molecule comprising a PDZ domain and the recognition unit;
where the compound is not a peptide.

80-83. (canceled)

84. The method of claim 79 wherein the molecule comprising a PDZ domain is selected from the group consisting of: PDZP1, PDZP2, PDZP3, PDZP4, PDZP5, PSD-95, Chapsyn, KIAA, SAP-90, hdlg, NJRF, TKA-1, NMDAR, nNOs, EAP-1, LCAF/IL-16, Ina D, ZO-1, ZO-2, p55, bSYN1, bSYN2, PTP-BAS, PTPH1/PTP-MEG, LIMK, MAST-205, Tlam, Af-6, Dsh, LCAF, NK/T-ZIP, Ros-1, RO1/H 10.8, F28FS, F54E7, and LIN-Z/CASK.

85. The method of claim 84 wherein the molecule comprising a PDZ domain is PSD-95.

86. The method of claim 85 wherein the PDZ domain of PSD-95 is selected from the group consisting of PDZ1 domain of PSD-95 (SEQ ID NO:109), PDZ2 domain of PSD95 (SEQ ID NO: 110), and PDZ3 domain of PSD-95 (SEQ ID NO:9).

87. The method of claim 79 wherein the recognition unit that selectively binds to the PDZ domain is selected from the group consisting of: K+-Channel, KV 1.4 (SEQ ID NO:49); FAS Receptor (SEQ ID NO:58); NMDA (NR2B), mouse (SEQ ID NO:42); NGF Receptor (SEQ ID NO:59); β1 Adrenoreceptor (SEQ ID NO:30); Serotonin (SEQ ID NO:31); VIP (SEQ ID NO:32); CRF (SEQ ID NO:33); Na+Channel (α) (SEQ ID NO:51); Orphan Receptor (SEQ ID NO:34); Ankyrin (SEQ ID NO:64); Fanconi anemia group C protein (SEQ ID NO:65); Glucose transporter (SEQ ID NO:56); β-1 Adrenergic (SEQ ID NO:35); Calcium pump (SEQ ID NO:66); BCR (SEQ ID NO:68); MPK2 (SEQ ID NO:69); HPV18, E6 (SEQ ID NO:37); HSV11, UL25 (SEQ ID NO:38); EBV, GP3 (SEQ ID NO:39); HTL1A, TAT (SEQ ID NO:40); VZVD, UL14 (SEQ ID NO:41); Somatostatin Receptor (Type2) (SEQ ID NO:61); Colorectal Mutant Cancer Protein (SEQ ID NO:70); Transmembrane Receptor (frizzled) (SEQ ID NO:53); Homologue of frizzled, rat (SEQ ID NO:54); Neurexin III, bovine (SEQ ID NO:73); Neurexin II, bovine (SEQ ID NO:74); and K+-Channel, Kir 2.2v (SEQ ID NO:50).

88. The method of claim 87 wherein the recognition unit that selectively binds to the PDZ domain is Na+Channel (a) (SEQ ID NO:51).

89. The method of claim 79 wherein the candidate compound is selected from the group consisting of: carbohydrate, oligonucleotide, and small drug molecule.
